(19) United States
(12) Patent Application Publication
(10) Pub. No.: US 2024/0343819 A1
(43) Pub. Date: Oct. 17, 2024

(54) METHODS OF TREATING MULTIPLE MYELOMA

(71) Applicants: Seagen Inc., Bothell, WA (US); SpringWorks Therapeutics, Inc., Stamford, CT (US)

(72) Inventors: Phoenix Ho, Shoreline, WA (US); Heather Alane Van Epps, Seattle, WA (US); Mary Campbell, Seattle, WA (US); Amy Kim, Emerald Hills, CA (US); Shinta Cheng, Stamford, CT (US); Todd Shearer, Stamford, CT (US)

(21) Appl. No.: 18/694,483

(22) PCT Filed: Sep. 20, 2022
(86) PCT No.: PCT/US2022/076694

§ 371 Date: Mar. 22, 2024

Related U.S. Application Data
(60) Provisional application No. 63/247,637, filed on Sep. 23, 2021.

(51) Int. Cl. C07K 16/28; A61K 31/4164; A61K 31/573; A61K 39/00; A61P 35/00

(52) U.S. Cl. CPC C07K 16/2878; A61K 31/4164; A61K 31/573; A61P 35/00; A61K 2039/505; A61K 2039/545

(57) ABSTRACT

Provided herein are methods of treating multiple myeloma (MM) using specific doses of an anti-B-cell migration antigen (BCMA) antibody and nirogacestat, and optionally, dexamethasone.

### CLAIM OF PRIORITY

This application claims the benefit of U.S. Provisional Application No. 63/247,637, filed on Sep. 23, 2021. The disclosure of the prior application is hereby incorporated by reference in its entirety.

### SEQUENCE LISTING

This application contains a Sequence Listing that has been submitted electronically as an XML filed named “49223-0060WO1_SL_ST26.XML.” The XML file, created on Sep. 13, 2022, is 19,519 bytes in size. The material in the XML file is hereby incorporated by reference in its entirety.

### BACKGROUND

Multiple Myeloma (MM) is a neoplastic disorder of clonally proliferating plasma cells in the bone marrow, peripheral blood, or other extramedullary sites. Malignant plasma cells exert a direct pathologic effect on the marrow microenvironment and adjacent skeletal bone, leading to anemia, osteolytic bone lesions, and hypercalcemia. In most cases, malignant plasma cells also produce an abnormal monoclonal immunoglobulin known as the M protein, but in a minority of subjects, the myeloma cells produce only monoclonal free light chains (FLC). Abnormal levels of either M protein or FLC can contribute to the clinical spectrum of disease that includes renal failure and an increased susceptibility to infections (Kumar et al., Nat. Rev. Dis. Primers 3:17046, 2017; Palumbo et al., N. Engl. J. Med. 364(11):1046-1060, 2011; Rollig et al., Lancet 385(9983):2197-2208, 2015).

Standard treatments for MM include combination chemotherapy regimens containing proteasome inhibitors (PIs) such as bortezomib and carfilzomib, and ixazomib, and/or immunomodulatory drugs (IMiDs), such as lenalidomide and pomalidomide. Alkylating agents such as melphalan and cyclophosphamide are also active in MM. Patients who are free from significant comorbidities and considered eligible, are often treated with myeloablative chemotherapy and/or radiation, followed by autologous stem cell transplant (ASCT) (Rollig et al., Lancet. 385(9983):2197-208, 2015; and Rajkumar et al., Mayo Clin Proc. 91(1):101-19, 2016). More recently, daratumumab, a monoclonal antibody targeting the CD38 antigen, has been approved for the treatment of RRMM as monotherapy in fourth line therapy.

To date, multiple myeloma remains an incurable disease managed with sequential lines of treatment that typically yield shorter durations of disease control with each subsequent relapse (Kumar et al., Mayo Clin. Proc. 79(7):867-874, 2004).

### SUMMARY

This application is based upon evidence demonstrating the efficacy of combining certain BCMA therapeutic agents such as BCMA antibodies, including non-fucosylated antibodies, with various other therapeutics to treat cancers such as MM. Therapeutics found to successfully combine with such BCMA agents (e.g., non-fucosylated antibodies) include nirogacestat and/or dexamethasone.

In another aspect, the application is based in part on the identification of various BCMA antibody dosing regimens, including a standard and an intensive dosing regimen (defined more fully below) that have been shown to be therapeutically efficacious in combination therapy, including combinations with nirogacestat and/or dexamethasone. These results were unexpected given that a relatively high level of a BCMA antibody as described herein could be administered while still maintaining a manageable safety profile, including even when the BCMA antibody was administered as part of a combination therapy.

Accordingly, provided herein are methods of treating a subject having multiple myeloma (MM) that include administering to the subject: (i) one or more doses of an antibody, or antigen-binding fragment thereof, that specifically binds to a B cell maturation antigen (BCMA), and (ii) one or more doses of nirogacestat, and wherein: the one or more doses of the antibody or antigen-binding fragment thereof are independently administered to the subject at about 100 mg of the antibody or antigen-binding fragment thereof to about 2,000 mg of the antibody or antigen-binding fragment thereof, and the one or more doses of nirogacestat are independently administered to the subject at about 80 mg to about 120 mg of nirogacestat.

In some embodiments of any of the methods described herein, the antibody or antigen-binding fragment thereof is a non-fucosylated antibody or antigen-binding fragment thereof.

In some embodiments of any of the methods described herein, a composition including the antibody or antigen-binding fragment thereof is administered to the subject, and about or at least 95%, 97%, 98% or 99% of the antibody or antigen-binding fragment thereof in the composition are afucosylated.

In some embodiments of any of the methods described herein, the antibody or antigen-binding fragment thereof, includes: a heavy chain variable region including a CDR1 including SEQ ID NO: 1, a CDR2 including SEQ ID NO: 2, and a CDR3 including SEQ ID NO: 3, and a light chain variable domain including a CDR1 including SEQ ID NO: 5, a CDR2 including SEQ ID NO: 6, and a CDR3 including SEQ ID NO: 7.

In some embodiments of any of the methods described herein, the antibody or the antigen-binding fragment thereof includes a heavy chain variable domain including an amino acid sequence that is at least 80% identical to SEQ ID NO: 4 and a light chain variable domain including an amino acid sequence that is at least 80% identical to SEQ ID NO: 8.

In some embodiments of any of the methods described herein, the antibody or the antigen-binding fragment thereof includes a heavy chain variable domain including an amino acid sequence that is at least 90% identical to SEQ ID NO: 4 and a light chain variable domain including an amino acid sequence that is at least 90% identical to SEQ ID NO: 8.

In some embodiments of any of the methods described herein, the antibody or the antigen-binding fragment thereof includes a heavy chain variable domain including an amino acid sequence of SEQ ID NO: 4 and a light chain variable domain including an amino acid sequence of SEQ ID NO: 8.

In some embodiments of any of the methods described herein, the antibody or the antigen-binding fragment thereof is humanized.

In some embodiments of any of the methods described herein, the antibody is an IgG1 antibody.

In some embodiments of any of the methods described herein, the antibody or antigen-binding fragment thereof is not a bispecific antibody, a bispecific T cell engager (BiTE), a chimeric antigen receptor (CAR), or an antibody drug conjugate (ADC), or a portion thereof.

In some embodiments of any of the methods described herein, the one or more doses of the antibody or the antigen-binding fragment thereof are independently administered to the subject at about 200 mg of the antibody or antigen-binding fragment thereof to about 1600 mg of the antibody or the antigen-binding fragment thereof.

In some embodiments of any of the methods described herein, the one or more doses of the antibody or the antigen-binding fragment thereof are independently administered to the subject at about 200 mg of the antibody or antigen-binding fragment thereof to about 800 mg of the antibody or the antigen-binding fragment thereof.

In some embodiments of any of the methods described herein, the one or more doses of the antibody or the antigen-binding fragment thereof are independently administered to the subject at about 400 mg of the antibody or antigen-binding fragment thereof to about 800 mg of the antibody or the antigen-binding fragment thereof.

In some embodiments of any of the methods described herein, the one or more doses of the antibody or the antigen-binding fragment thereof are independently administered to the subject at about 100 mg of the antibody or antigen-binding fragment thereof to about 400 mg of the antibody or the antigen-binding fragment thereof.

In some embodiments of any of the methods described herein, the one or more doses of the antibody or the antigen-binding fragment thereof are independently administered to the subject at about 800 mg of the antibody or antigen-binding fragment thereof to about 2,000 mg of the antibody or antigen-binding fragment thereof.

In some embodiments of any of the methods described herein, the one or more doses of the antibody or the antigen-binding fragment thereof are independently administered to the subject at about 1,200 mg of the antibody or antigen-binding fragment thereof to about 2,000 mg of the antibody or antigen-binding fragment thereof.

In some embodiments of any of the methods described herein, the one or more doses of the antibody or the antigen-binding fragment thereof are independently administered to the subject at about 1,400 mg of the antibody or antigen-binding fragment thereof to about 1,800 mg of the antibody or antigen-binding fragment thereof.

In some embodiments of any of the methods described herein, the one or more doses of the antibody or the antigen-binding fragment thereof are independently administered to the subject at about 100 mg of the antibody or antigen-binding fragment thereof.

In some embodiments of any of the methods described herein, the one or more doses of the antibody or the antigen-binding fragment thereof are independently administered to the subject at about 200 mg of the antibody or antigen-binding fragment thereof.

In some embodiments of any of the methods described herein, the one or more doses of the antibody or the antigen-binding fragment thereof are independently administered to the subject at about 400 mg of the antibody or antigen-binding fragment thereof.

In some embodiments of any of the methods described herein, the one or more doses of the antibody or the antigen-binding fragment thereof are independently administered to the subject at about 800 mg of the antibody or antigen-binding fragment thereof.

In some embodiments of any of the methods described herein, the one or more doses of the antibody or the antigen-binding fragment thereof are administered to the subject at about 1,600 mg of the antibody or antigen-binding fragment thereof.

In some embodiments of any of the methods described herein, a single dose of the antibody or antigen-binding fragment thereof is administered to the subject.

In some embodiments of any of the methods described herein, two or more doses of the antibody or antigen-binding fragment thereof are independently administered to the subject.

In some embodiments of any of the methods described herein, the two or more doses of the antibody or the antigen-binding fragment thereof are independently administered to the subject at a frequency of between once a week and about once every four weeks.

In some embodiments of any of the methods described herein, the two or more doses of the antibody or the antigen-binding fragment thereof are independently administered to the subject at a frequency of about once a week.

In some embodiments of any of the methods described herein, the two or more doses of the antibody or the antigen-binding fragment thereof are independently administered to the subject at a frequency of about once every two weeks.

In some embodiments of any of the methods described herein, the two or more doses of the antibody or the antigen-binding fragment thereof are independently administered to the subject at a frequency of about once every three weeks.

In some embodiments of any of the methods described herein, the two or more doses of the antibody or the antigen-binding fragment thereof are independently administered to the subject at a frequency of about once every four weeks.

In some embodiments of any of the methods described herein, each dose of the antibody or the antigen-binding fragment thereof includes about 100 mg of the antibody or the antigen-binding fragment thereof and is independently administered to the subject about once a week or about once every 2 weeks.

In some embodiments of any of the methods described herein, each dose of the antibody or the antigen-binding fragment thereof includes about 200 mg of the antibody or the antigen-binding fragment thereof and is independently administered to the subject about once a week or about once every 2 weeks.

In some embodiments of any of the methods described herein, each dose of the antibody or the antigen-binding fragment thereof includes about 400 mg of the antibody or the antigen-binding fragment thereof and is independently administered to the subject about once a week or about once every 2 weeks.

In some embodiments of any of the methods described herein, each dose of the antibody or the antigen-binding fragment thereof includes about 800 mg of the antibody or antigen-binding fragment thereof and is independently administered to the subject about once a week or about once every 2 weeks.

In some embodiments of any of the methods described herein, each dose of the antibody or the antigen-binding fragment thereof includes about 1600 mg of the antibody or antigen-binding fragment thereof and is independently administered to the subject about once a week or about once every 2 weeks.

In some embodiments of any of the methods described herein, individual doses of the antibody or antigen-binding fragment thereof are independently administered to the subject on day 1 and day 15 of a 28-day cycle.

In some embodiments of any of the methods described herein, individual doses of the antibody or antigen-binding fragment thereof are independently administered to the subject on day 1, day 8, day 15, and day 22 of a 28-day cycle.

In some embodiments of any of the methods described herein, the individual doses of the antibody or antigen-binding fragment thereof are independently administered to the subject for multiple 28-day cycles.

In some embodiments of any of the methods described herein, the two or more doses of the antibody or the antigen-binding fragment thereof include (1) one or more induction doses that are independently administered to the subject during an induction phase and (2) one or more maintenance doses of the antibody or the antigen-binding fragment thereof that are independently administered to the subject during a maintenance phase after the induction phase.

In some embodiments of any of the methods described herein, a single induction dose is administered to the subject.

In some embodiments of any of the methods described herein, two or more induction doses are independently administered to the subject.

In some embodiments of any of the methods described herein, each of the two or more induction doses are independently administered to the subject about once a week for about 1-10 weeks.

In some embodiments of any of the methods described herein, each of the two or more induction doses are independently administered to the subject once a week for 8 weeks.

In some embodiments of any of the methods described herein, induction doses are independently administered to the subject 4 times within a 28-day cycle.

In some embodiments of any of the methods described herein, induction doses are independently administered to the subject 8 times within two 28-day cycles.

In some embodiments of any of the methods described herein, individual induction doses are independently administered to the subject on day 1, day 8, day 15 and day 22 for each of the two 28-day cycles.

In some embodiments of any of the methods described herein, each of the induction dose(s) include(s) about 100, about 200, about 400, about 800, or about 1600 mg of the antibody or antigen-binding fragment thereof.

In some embodiments of any of the methods described herein, each induction dose includes about 800 mg of the antibody or antigen-binding fragment thereof.

In some embodiments of any of the methods described herein, each induction dose includes about 1600 mg of the antibody or antigen-binding fragment thereof.

In some embodiments of any of the methods described herein, a single maintenance dose is administered to the subject.

In some embodiments of any of the methods described herein, two or more maintenance doses are independently administered to the subject.

In some embodiments of any of the methods described herein, each of the two or more maintenance doses are independently administered to the subject once every 1-4 weeks.

In some embodiments of any of the methods described herein, each of the two or more maintenance doses are independently administered to the subject once every two weeks.

In some embodiments of any of the methods described herein, individual maintenance doses are independently administered to the subject on day 1 and day 15 of a 28-day cycle.

In some embodiments of any of the methods described herein, each maintenance dose includes about 100, about 200, about 400, about 800, or about 1600 mg of the antibody or antigen-binding fragment thereof.

In some embodiments of any of the methods described herein, each maintenance dose includes about 800 mg of the antibody or antigen-binding fragment thereof.

In some embodiments of any of the methods described herein, each maintenance doses includes about 1600 mg of the antibody or antigen-binding fragment thereof.

In some embodiments of any of the methods described herein, the antibody or antigen-binding fragment thereof is dosed q1wk during the induction phase for a total of 8 induction phase doses and dosed q2wk during the maintenance phase.

In some embodiments of any of the methods described herein, each induction dose includes about 100, about 200, about 400, about 800, or about 1600 mg of the antibody or antigen-binding fragment thereof, each maintenance dose includes about 100, about 200, about 400, about 800, or about 1600 mg of the antibody or antigen-binding fragment thereof, the individual induction doses are independently administered to the subject on each of day 1, day 8, day 15 and day 22 for each of two 28-day cycles for a total of 8 induction doses during the induction phase; and the individual maintenance doses are independently administered to the subject on each of days 1 and day 15 of each of one or more subsequent 28-day cycle(s).

In some embodiments of any of the methods described herein, each induction dose and each maintenance dose includes about 800 or about 1600 mg of the antibody or antigen-binding fragment thereof.

In some embodiments of any of the methods described herein, each induction dose and each maintenance dose includes about 1600 mg of the antibody or antigen-binding fragment thereof.

In some embodiments of any of the methods described herein, the dose(s) of the antibody or antigen-binding fragment thereof are administered intravenously to the subject.

In some embodiments of any of the methods described herein, a single dose of nirogacestat is administered to the subject.

In some embodiments of any of the methods described herein, two or more doses of nirogacestat are independently administered to the subject.

In some embodiments of any of the methods described herein, each dose of nirogacestat includes about 100 mg of nirogacestat.

In some embodiments of any of the methods described herein, the two or more doses of nirogacestat are independently administered to the subject at a frequency of about once a day to about four times a day.

In some embodiments of any of the methods described herein, the two or more doses of nirogacestat are independently administered to the subject at a frequency of about twice a day.

In some embodiments of any of the methods described herein, each of the two or more doses of nirogacestat includes about 100 mg of nirogacestat and the two or more doses of nirogacestat are independently administered to the subject at a frequency of about twice a day, each day of one or more 28-day cycle(s).

In some embodiments of any of the methods described herein, the dose(s) of nirogacestat is/are orally administered to the subject.

In some embodiments of any of the methods described herein, the method further includes independently administering one or more doses of dexamethasone to the subject.

In some embodiments of any of the methods described herein, the method includes administering a single dose of dexamethasone to the subject.

In some embodiments of any of the methods described herein, the method further includes independently administering two or more doses of dexamethasone to the subject.

In some embodiments of any of the methods described herein, the two or more doses of dexamethasone are independently administered to the subject at a frequency of about once a week.

In some embodiments of any of the methods described herein, each dose of dexamethasone includes about 30 mg to about 50 mg of dexamethasone.

In some embodiments of any of the methods described herein, each dose of dexamethasone includes about 40 mg of dexamethasone.

In some embodiments of any of the methods described herein, each dose of dexamethasone is/are intravenously administered to the subject.

In some embodiments of any of the methods described herein, when a dose of dexamethasone and a dose of the antibody or antigen-binding fragment thereof are administered to the subject on the same day, the dose of dexamethasone is administered to the subject about 1 to about 3 hours before the dose of the antibody or antigen-binding fragment thereof is administered to the subject.

In some embodiments of any of the methods described herein, each of two or more doses of the antibody or antigen-binding fragment thereof are independently administered to the subject at a frequency of about once every 1-4 weeks; each of the two or more doses of nirogacestat are independently administered to the subject at a frequency of once a day to about four times a day; and each of the two or more doses of dexamethasone are independently administered to the subject at a frequency of about once every 1-4 weeks.

In some embodiments of any of the methods described herein, each of the two or more doses of the antibody or antigen-binding fragment thereof are independently administered to the subject about once every two weeks; each of the two or more doses of nirogacestat are independently administered to the subject twice a day; and each of the two or more doses of dexamethasone are independently administered to the subject about once a week.

In some embodiments of any of the methods described herein, each of the two or more doses of the antibody or antigen-binding fragment thereof are independently administered to the subject on each of day 1 and day 15 of one or more 28-day cycle(s); each of the two or more doses of nirogacestat are independently administered to the subject on each of day 1 to day 28 of the one or more 28-day cycle(s); and each of the two or more doses of dexamethasone are independently administered to the subject on each of day 1, day 8, day 15 and day 22 of the one or more 28-day cycle(s).

In some embodiments of any of the methods described herein, each of the two or more doses of the antibody or antigen-binding fragment includes about 400 to about 1,600 mg of the antibody or antigen-binding fragment thereof, each of the two or more doses of nirogacestat includes about 100 mg of nirogacestat, and each of the two or more doses of dexamethasone includes about 40 mg of dexamethasone.

In some embodiments of any of the methods described herein, each of the two or more doses of the antibody or antigen-binding fragment includes about 400 mg of the antibody or antibody or antigen-binding fragment thereof, each of the two or more doses of nirogacestat includes about 100 mg of nirogacestat, and each of the two or more doses of dexamethasone includes about 40 mg of dexamethasone.

In some embodiments of any of the methods described herein, each of the two or more doses of the antibody or antigen-binding fragment includes about 800 mg of the antibody or antibody or antigen-binding fragment thereof, each of the two or more doses of nirogacestat includes about 100 mg of nirogacestat, and each of the two or more doses of dexamethasone includes about 40 mg of dexamethasone.

In some embodiments of any of the methods described herein, each of the two or more doses of the antibody or antigen-binding fragment includes about 1,600 mg of the antibody or antibody or antigen-binding fragment thereof, each of the two or more doses of nirogacestat includes about 100 mg of nirogacestat, and each of the two or more doses of dexamethasone includes about 40 mg of dexamethasone.

In some embodiments of any of the methods described herein, two or more doses of the antibody or antigen-binding fragment thereof are independently administered to the subject at a frequency of about once a week during an induction phase, and two or more doses of the antibody or antigen-binding fragment thereof are independently administered to the subject at a frequency of about once every two weeks during a subsequent maintenance phase; two or more doses of nirogacestat are independently administered to the subject at a frequency of about twice a day during one or both of the induction phase and the maintenance phase; and two or more doses of dexamethasone are independently administered to the subject at a frequency of about once a week during one or both of the induction phase and the maintenance phase.

In some embodiments of any of the methods described herein, the induction phase is about 8 weeks.

In some embodiments of any of the methods described herein, two or more doses of the antibody or antigen-binding fragment thereof are independently administered to the subject on each of day 1, day 8, day 15, and day 22 of each of two 28-day cycles of the induction phase and then on each of day 1 and day 15 of subsequent 28-day cycle(s) of the maintenance phase; two or more doses of nirogacestat are independently administered to the subject on each of day 1 to day 28 of each of the two 28-day cycles of the induction phase and each of the subsequent 28-day cycle(s) of the maintenance phase; and two or more doses of dexamethasone are independently administered to the subject on each of day 1, day 8, day 15, and day 22 of each of the two 28-day cycles of the induction phase and each of the subsequent 28-day cycle(s) of the maintenance phase.

In some embodiments of any of the methods described herein, the two or more doses of the antibody or antigen-binding fragment independently administered to the subject on each of day 1, day 8, day 15, and day 22 of each of the two 28-day cycles of the induction phase includes about 100 mg, about 200 mg, about 400 mg, about 800 mg or about 1600 mg of the antibody or antigen-binding fragment thereof, the two or more doses of the antigen or antigen-binding fragment independently administered to the subject on each of day 1 and day 15 of each of the subsequent 28-day cycle(s) of the maintenance phase includes about 100, about 200, about 400, about 800, or about 1600 mg; the two or more doses of nirogacestat independently administered to the subject on each of day 1 to day 28 of each of the two 28-day cycles of the induction phase and each of the subsequent 28-day cycle(s) of the maintenance phase includes about 80 mg to about 120 mg of nirogacestat; and the two or more doses of dexamethasone independently administered to the subject on each of day 1, day 8, day 15, and day 22 of each of the two 28-day cycles of the induction phase and each of the subsequent 28-day cycle(s) of the maintenance phase includes about 20 mg to about 60 mg of dexamethasone.

In some embodiments of any of the methods described herein, the two or more doses of the antibody or antigen-binding fragment thereof independently administered to the subject on each of day 1, day 8, day 15, and day 22 of each of the two 28-day cycles of the induction phase includes about 800 mg of the antibody or antigen-binding fragment thereof.

In some embodiments of any of the methods described herein, the two or more doses of the antibody or antigen-binding fragment thereof independently administered to the subject on each of day 1, day 8, day 15, and day 22 of each of the two 28-day cycles of the induction phase includes about 1,600 mg of the antibody or antigen-binding fragment thereof.

In some embodiments of any of the methods described herein, the two or more doses of nirogacestat independently administered to the subject on each of day 1 to day 28 of each of the two 28-day cycles of the induction phase and each of the subsequent 28-day cycle(s) of the maintenance phase includes about 100 mg of nirogacestat.

In some embodiments of any of the methods described herein, the two or more doses of dexamethasone independently administered to the subject on each of day 1, day 8, day 15, and day 22 of each of the two 28-day cycles of the induction phase and each of the subsequent 28-day cycle(s) of the maintenance phase includes about 20 mg dexamethasone.

In some embodiments of any of the methods described herein, the two or more doses of dexamethasone independently administered to the subject on each of day 1, day 8, day 15, and day 22 of each of the two 28-day cycles of the induction phase and each of the subsequent 28-day cycle(s) of the maintenance phase includes about 40 mg dexamethasone.

In some embodiments of any of the methods described herein, the two or more doses of the antibody or antigen-binding fragment independently administered to the subject on each of day 1, day 8, day 15, and day 22 of each of the two 28-day cycles of the induction phase includes about 1600 mg of the antibody or antigen-binding fragment thereof, the two or more doses of the antigen or antigen-binding fragment independently administered to the subject on each of day 1 and day 15 of each of the subsequent 28-day cycle(s) of the maintenance phase includes about 1600 mg; the two or more doses of nirogacestat independently administered to the subject twice a day on each of day 1 to day 28 of each of the two 28-day cycles of the induction phase and each of the subsequent 28-day cycle(s) of the maintenance phase includes about 100 mg of nirogacestat; and the two or more doses of dexamethasone independently administered to the subject on each of day 1, day 8, day 15, and day 22 of each of the two 28-day cycles of the induction phase and each of the subsequent 28-day cycle(s) of the maintenance phase includes about 40 mg of dexamethasone.

In some embodiments of any of the methods described herein, the two or more doses of the antibody or antigen-binding fragment independently administered to the subject on each of day 1, day 8, day 15, and day 22 of each of the two 28-day cycles of the induction phase includes about 800 mg of the antibody or antigen-binding fragment thereof, the two or more doses of the antigen or antigen-binding fragment independently administered to the subject on each of day 1 and day 15 of each of the subsequent 28-day cycle(s) of the maintenance phase includes about 800 mg; the two or more doses of nirogacestat independently administered to the subject twice a day on each of day 1 to day 28 of each of the two 28-day cycles of the induction phase and each of the subsequent 28-day cycle(s) of the maintenance phase includes about 100 mg of nirogacestat; and the two or more doses of dexamethasone independently administered to the subject on each of day 1, day 8, day 15, and day 22 of each of the two 28-day cycles of the induction phase and each of the subsequent 28-day cycle(s) of the maintenance phase includes about 40 mg of dexamethasone.

In some embodiments of any of the methods described herein, the two or more doses of dexamethasone are administered to the subject by intravenous administration.

In some embodiments of any of the methods described herein, the two or more doses of the antibody or antigen-binding fragment thereof are administered to the subject by intravenous administration.

In some embodiments of any of the methods described herein, at least an initial dose of the two or more doses of the antibody or antigen-binding fragment thereof is administered to the subject using step-wise infusion.

In some embodiments of any of the methods described herein, the step-wise infusion is performed using an infusion rate of about 50 mg/hour to about 400 mg/hour.

In some embodiments of any of the methods described herein, during the step-wise infusion, the infusion rate is increased every 30 minutes.

In some embodiments of any of the methods described herein, during the step-wise infusion, the infusion rate is increased no more than two-fold every 30 minute.

In some embodiments of any of the methods described herein, the two or more doses of the nirogacestat are administered to the subject by oral administration.

In some embodiments of any of the methods described herein, the subject is a human subject.

In some embodiments of any of the methods described herein, the subject has previously been diagnosed as having multiple myeloma.

In some embodiments of any of the methods described herein, the subject has relapsed or refractory multiple myeloma.

In some embodiments of any of the methods described herein, the subject was previously administered one or more therapeutic agents or treatments for multiple myeloma.

In some embodiments of any of the methods described herein, the previously administered one or more therapeutic agents or treatments for multiple myeloma were unsuccessful.

In some embodiments of any of the methods described herein, the subject has previously been administered at least one of a proteasome inhibitor, an immunomodulatory agent, and an anti-CD38 antibody, or cannot tolerate any of the foregoing.

In some embodiments of any of the methods described herein, the subject has previously been administered therapeutic agents including all three of a proteasome inhibitor, an immunomodulatory agent, and an anti-CD38 antibody, or cannot tolerate any of the foregoing.

In some embodiments of any of the methods described herein, the subject has previously been administered at least three prior lines of anti-multiple myeloma therapy and is refractory to at least one therapeutic agent in each of the following classes: a proteasome inhibitor, an immunomodulatory agent, and an anti-CD38 antibody.

In some embodiments of any of the methods described herein, the subject has previously been administered a BCMA-directed myeloma therapy other than the antibody or antigen-binding fragment thereof.

In some embodiments of any of the methods described herein, the subject satisfies 1, 2 or all 3 of the following criteria prior to initiating treatment: (1) serum monoclonal paraprotein (M-protein) level of ≥0.5 g/dL, urine M-protein level ≥200 mg/24 hr, (2) serum immunoglobulin free light chain ≥10 mg/dL, and/or (3) abnormal serum immunoglobulin kappa lambda free light chain ratio.

In some embodiments of any of the methods described herein, the method results in a steady-state concentration of the antibody or antigen-binding fragment thereof, in the serum of the subject of about 1 μg/mL to about 200 μg/mL.

In some embodiments of any of the methods described herein, the method results in a steady-state concentration of free light chain (FLC) in the serum of the subject of less than 50 mg/dL.

In some embodiments of any of the methods described herein, the subject has received at least two prior lines of anti-multiple myeloma therapy and/or has documented IMWG (International Myeloma Working Group) disease progression on or within 60 days of completion of the two prior lines of antimyeloma therapy.

In some embodiments of any of the methods described herein, or more therapeutic effects in the subject is improved after administration of the dose(s) of the antibody or antigen-binding fragment thereof, the dose(s) of nirogacestat, and optionally, the dose(s) of dexamethasone, relative to a baseline.

In some embodiments of any of the methods described herein, the one or more therapeutic effects is selected from the group consisting of: objective response rate, complete response rate, duration of response, duration of complete response, time to response, progression free survival, and overall survival of the subject.

In some embodiments of any of the methods described herein, the objective response rate is at least about 20%, at least about 25%, at least about 30%, at least about 35%, at least about 40%, at least about 45%, at least about 50%, at least about 60%, at least about 70%, or at least about 80%.

In some embodiments of any of the methods described herein, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years.

In some embodiments of any of the methods described herein, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years.

In some embodiments of any of the methods described herein, the duration of response or the duration of complete response to the administration is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years.

Also provided are kits including: (a) one or more doses of a pharmaceutical composition including an antibody, or antigen-binding fragment thereof, that specifically binds to a B cell maturation antigen (BCMA), wherein the antibody or antigen-binding fragment thereof, includes: a heavy chain variable region including a CDR1 including SEQ ID NO: 1, a CDR2 including SEQ ID NO: 2, and a CDR3 including SEQ ID NO: 3, and a light chain variable domain including a CDR1 including SEQ ID NO: 5, a CDR2 including SEQ ID NO: 6, and a CDR3 including SEQ ID NO: 7; and (b) instructions for performing any of the methods described herein.

In some embodiments of any of the kits described herein, the kit further includes one or more doses of a pharmaceutical composition including nirogacestat. In some embodiments of any of the kits described herein, the kit further includes one or more doses of a pharmaceutical composition including dexamethasone.

Unless otherwise defined, all technical and scientific terms used herein have the same meaning as commonly understood by one of ordinary skill in the art to which this invention belongs. Methods and materials described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic showing continuation or discontinuation of treatment with nirogacestat.

FIG. 2A. NCI-H929 cells displayed increased BCMA expression upon DAPT treatment. Light gray: isotype control; medium gray: untreated cells; dark gray: DAPT treated cells.

FIG. 2B. Molp-8 cells displayed increased BCMA expression upon DAPT treatment. Light gray: isotype control; medium gray: untreated cells; dark gray: DAPT treated cells.

FIG. 2C. Fold over background of NFAT signaling due to FcγRIII engagement. DAPT (GSI) treated NCI-H929 cells compared to untreated cells (N=3).

FIG. 2D. Fold over background of NFAT signaling due to FcγRIII engagement. DAPT (GSI) treated Molp-8 cells compared to untreated cells (N=3).

FIG. 3A Overnight treatment of multiple myeloma cells with nirogacestat induced BCMA expression in target cells.

FIG. 3B Increase in multiple myeloma target cell lysis mediated by isolated primary natural killer (NK) cells (MOLP-8 cells incubated with high affinity FcγRIII V/V genotype donor cells and U266 with low affinity FcγRIII V/F genotype donor cells).

FIG. 4A. Molp-8 cells displayed increased BCMA expression upon Nirogacestat treatment. Dark gray: isotype control; medium gray: untreated cells; light gray: Nirogacestat treated cells.

FIG. 4B. The maximum percentage of target cell lysis of Nirogacestat treated cells compared to untreated cells (N=3). hIgG1k is a non-binding antibody control.

FIG. 5. p65 activation of NCI-H929 cells bound with and without SEA-BCMA, treated with and without APRIL, in the presence or absence of Nirogacestat.

### DETAILED DESCRIPTION

Provided herein are methods of treating a subject having multiple myeloma (MM) that comprise administering to the subject (i) one or more doses of an antibody that binds to B cell maturation antigen (BCMA), or antigen-binding fragment thereof, and (ii) one or more doses of nirogacestat, wherein: the one or more doses of the antibody or antigen-binding fragment thereof are independently administered to the subject at about 100 mg of the antibody or antigen-binding fragment thereof to about 2,000 mg of the antibody or antigen-binding fragment thereof, and the one or more doses of nirogacestat are independently administered to the subject at about 80 mg to about 120 mg of nirogacestat. In some embodiments, the method further includes administering one or more doses of dexamethasone.

In some embodiments, the antibody is an IgG1 antibody. In some embodiments, the antibody is an afucosylated antibody. In some embodiments, the antibody or antigen-binding fragment thereof, comprises: a heavy chain variable region comprising a CDR1 comprising SEQ ID NO: 1, a CDR2 comprising SEQ ID NO: 2, and a CDR3 comprising SEQ ID NO: 3, and a light chain variable domain comprising a CDR1 comprising SEQ ID NO: 5, a CDR2 comprising SEQ ID NO: 6, and a CDR3 comprising SEQ ID NO: 7. In some embodiments, one or more doses of 1600 mg of the antibody, or antigen-binding fragment thereof, is independently administered to the subject at a frequency of every two weeks. In some embodiments, one or more doses of 800 mg of the antibody, or antigen-binding fragment thereof, is independently administered to the subject at a frequency of every week. In some embodiments, about 1-2 induction doses of 1600 mg of the antibody, or antigen-binding fragment thereof, is independently administered to the subject at a frequency of every week, followed by one or more maintenance doses of 1600 mg of the antibody, or antigen-binding fragment thereof, independently administered to the subject at a frequency of every two weeks. In some embodiments, about 1-2 induction doses of 800 mg of the antibody, or antigen-binding fragment thereof, is independently administered to the subject at a frequency of every week, followed by one or more maintenance doses of 1600 mg of the antibody, or antigen-binding fragment thereof, independently administered to the subject at a frequency of every two weeks.

In some embodiments, the multiple myeloma is relapsed or refractory multiple myeloma (RRMM). In some embodiments, the subject was previously administered one or more therapeutic agents or treatments for multiple myeloma. The one or more previously administered therapeutic agents or treatments for multiple myeloma include, but are not limited to, a proteasome inhibitor (PI), an immunomodulatory drug (IMiD), and an anti-CD38 antibody. In some embodiments, the subject was previously administered at least one BCMA-directed myeloma therapy selected from the group consisting of: ADC, CAR-T cell therapy, and bispecific antibodies. In some embodiments, the one or more previously administered therapeutic agents or treatments were not effective in treating the multiple myeloma. In some embodiments, the subject has one or more (e.g., two, three, or four) of: a serum monoclonal paraprotein (M-protein) level of ≥0.5 g/dL, a urine M-protein level of ≥200 mg/24 hours, a serum immunoglobulin free light chain ≥10 mg/dL and/or an abnormal serum immunoglobulin kappa to lambda free light chain ratio.

In some embodiments, these methods result in, e.g., one or more of: a therapeutically desired steady-state concentration of an anti-BCMA antibody in the serum of a subject, a therapeutically desired reduction in the steady-state levels of free light chain in the serum of a subject, and a therapeutically desired saturation of BCMA in a subject.

Initial results from a clinical trial conducted with an afucosylated anti-BCMA antibody such as the SEA-BCMA antibody described herein show that the SEA-BCMA antibody can be administered at high doses (e.g., 800 mg or 1600 mg per dose) while still maintaining a tolerable safety profile. These initial results indicate that such an antibody can potentially be administered in flexible dosing regimens, including standard or intensive dosing regimens. The ability to dose at a high level also indicates that the antibody is a good candidate for dosing in combination with other therapeutic agents, including, for example, nirogacestat and/or dexamethasone.

### Multiple Myeloma

Multiple Myeloma (MM) is a neoplastic disorder of clonally proliferating plasma cells in the bone marrow, peripheral blood, or other extramedullary sites. Diagnosis of MM requiring systemic therapy is defined by International Myeloma Working Group (IMWG) 2014 criteria (Kumar 2016). Malignant plasma cells exert a direct pathologic effect on the marrow microenvironment and adjacent skeletal bone, leading to anemia, osteolytic bone lesions, and hypercalcemia. In most cases, malignant plasma cells also produce an abnormal monoclonal immunoglobulin known as the M protein, but in a minority of patients, the myeloma cells produce only monoclonal free light chains (FLC). Abnormal levels of either M protein or FLC can contribute to the clinical spectrum of disease that includes renal failure and an increased susceptibility to infections.

Standard treatments for multiple myeloma include combination chemotherapy regimens containing proteasome inhibitors (PIs), such as bortezomib and carfilzomib, and/or immunomodulatory drugs (IMiDs), such as lenalidomide and pomalidomide, together with corticosteroids. Alkylating agents such as melphalan and cyclophosphamide are also active in multiple myeloma. Patients who are free from significant comorbidities and considered eligible, are often treated with myeloablative chemotherapy and/or radiation. More recently, daratumumab, a monoclonal antibody targeting the CD38 antigen, has been approved for the treatment of RRMM as monotherapy in fourth line and in combination with bortezomib, lenalidomide, or pomalidomide plus dexamethasone in earlier lines of therapy based on significant clinical efficacy. Subsequently between 2018 and 2019, daratumumab garnered US Food Drug Administration approval for subjects with newly diagnosed multiple myeloma in combination with several standard of care (SOC) regiments (bortezomib+melphalan+prednisone, and lenalidomide+dexamethasone, for transplant-ineligible patients, and bortezomib+thalidomide+dexamethasone in transplant-eligible patients).

Conventional therapy of multiple myeloma (MM), such as combination chemotherapy regimens is not curative and most of the patients ultimately progress. In addition, some patients will not respond to initial treatment.

Duration of initial disease response remains one of the strongest prognostic factors in MM, particularly post autologous stem cell transplantation (ASCT). Early relapse (<24 months) after upfront ASCT strongly predicts lower overall survival (OS), and despite all advancements in the last two decades, the natural history of the disease remains grossly unchanged with the proportion of early relapses stable at around 35-38% (see, Kumar et al., Leukemia 32:986-95, 2018). These relapses usually present aggressively, with similar dismal outcomes from refractory disease, defined as progression under treatment or within 60 days after treatment cessation. Early relapses also do not allow for proper patient recovery from initial treatments and can severely limit treatment choices.

Almost all, if not all, myeloma patients eventually relapse, but while early relapses are usually aggressive and dismal, late relapses (>24 months) generally have a more indolent course. In addition, patients would usually have had time to recover, with little residual toxicity from previous interventions allowing more aggressive approaches. A high unmet need remains in later lines of therapy. The unmet need is pronounced in patients who are refractory to previous administered treatments of PIs, IMiDs, and anti-CD38 antibodies (“triple-class” refractory subjects).

Provided herein are methods of treating a subject having a multiple myeloma (MM). In some embodiments, the multiple myeloma is selected from the group consisting of a precursor to myeloma, multiple myeloma cancers which produce light chains of kappa-type and/or light chains of lambda-type, aggressive multiple myeloma, refractory multiple myeloma, and drug-resistant multiple myeloma. In some embodiments, the multiple myeloma is a relapsed or refractory multiple myeloma (RRMM). In some embodiments, the subject has one or more (e.g., two, three, or four) of: a serum monoclonal paraprotein (M-protein) level of ≥0.5 g/dL, a urine M-protein level of ≥200 mg/24 hours, a serum immunoglobulin free light chain ≥10 mg/dL, and/or an abnormal serum immunoglobulin kappa to lambda free light chain ratio.

Methods for assessing the efficacy of treatment in a subject having multiple myeloma include the measurement of free light chain, M protein, the level of hypercalcemia, and the relative number of myeloma cells in the subject.

### BCMA

B-cell maturation antigen (BCMA or BCM), also known as tumor necrosis factor receptor superfamily member 17 (TNFRSF17), is a protein that in humans is encoded by the TNFRSF17 gene. BCMA is an established plasmablast- and plasma cell-specific protein that mediates cell proliferation and survival. BCMA is expressed at moderate to low levels on the majority of MM patient tumor cells (Novak et al., Blood 103(2):689-694, 2004; Seckinger et al., Cancer Cell 31(3):396-410, 2017). The ligands APRIL and BAFF bind to BCMA and mediate pro-survival cellular signals (Moreaux et al., Blood 103(8):3148-3157, 2004; Novak et al., Blood 103(2):689-694, 2004; O'Connor et al., J. Exp. Med. 199(1):91-8, 2004).

Unless otherwise indicated, BCMA means a human BCMA. Exemplary sequences for wildtype human BCMA protein and wildtype human BCMA cDNA are shown below.

Wildtype Mature Human BCMA Protein
(SEQ ID NO: 9)
MLQMAGQCSQNEYFDSLLHACIPCQLRCSSNTPPLTCQRYCNASVTNSVKGTNAILWTC
LGLSLIISLAVFVLMFLLRKINSEPLKDEFKNTGSGLLGMANIDLEKSRTGDEIILPRGLEY
TVEECTCEDCIKSKPKVDSDHCFPLPAMEEGATILVTTKTNDYCKSLPAALSATEIEKSIS
AR
Wildtype Human BCMA cDNA
(SEQ ID NO: 10)
aagactcaaa cttagaaact tgaattagat gtggtattca aatccttagc tgccgcgaag
acacagacag cccccgtaag aacccacgaa gcaggcgaag ttcattgttc tcaacattct
agctgctctt gctgcatttg ctctggaatt cttgtagaga tattacttgt ccttccaggc
tgttctttct gtagctccct tgttttcttt ttgtgatcat gttgcagatg gctgggcagt
gctcccaaaa tgaatatttt gacagtttgt tgcatgcttg cataccttgt caacttcgat
gttcttctaa tactcctcct ctaacatgtc agcgttattg taatgcaagt gtgaccaatt
cagtgaaagg aacgaatgcg attctctgga cctgtttggg actgagctta ataatttctt
tggcagtttt cgtgctaatg tttttgctaa ggaagataaa ctctgaacca ttaaaggacg
agtttaaaaa cacaggatca ggtctcctgg gcatggctaa cattgacctg gaaaagagca
ggactggtga tgaaattatt cttccgagag gcctcgagta cacggtggaa gaatgcacct
gtgaagactg catcaagagc aaaccgaagg tcgactctga ccattgcttt ccactcccag
ctatggagga aggcgcaacc attcttgtca ccacgaaaac gaatgactat tgcaagagcc
tgccagctgc tttgagtgct acggagatag agaaatcaat ttctgctagg taattaacca
tttcgactcg agcagtgcca ctttaaaaat cttttgtcag aatagatgat gtgtcagatc
tctttaggat gactgtattt ttcagttgcc gatacagctt tttgtcctct aactgtggaa
actctttatg ttagatatat ttctctaggt tactgttggg agcttaatgg tagaaacttc
cttggtttca tgattaaact cttttttttc ctga

Unless otherwise apparent from the context reference to BMCA means at least an extracellular domain of a BCMA protein. An exemplary extracellular domain of human BCMA protein comprises amino acids 1 to 54 of SEQ ID NO: 9). In some embodiments, the anti-BCMA antibody or antigen-binding fragment described herein can bind specifically to BCMA expressed on the surface of a cancer cell (e.g., myeloma cell).

### Antibodies and Antigen-Binding Fragments

The term “antibody” is used herein in its broadest sense and includes proteins (e.g., single-chain polypeptides or multi-chain polypeptides) that comprise one or more antigen-binding domains that specifically bind to an antigen or epitope. An intact antibody usually comprises four polypeptides—two heavy chains and two light chains that are joined to form a “Y” shaped molecule. The amino acid sequence in the tips of the “Y” varies greatly among different antibodies. This variable region, composed of, for example, 110-130 amino acids, give the antibody its specificity for binding antigen. The variable region includes the ends of the light and heavy chains. Treating the antibody with a protease can cleave this region, producing Fab or antigen-binding fragment that include the variable ends of an antibody. The regions in the variable region that directly contact a portion of the antigen's surface are complementarity determining regions (CDRs). The light chain variable region (VL) and heavy chain variable region (VH) each comprises three CDRs—CDR1, CDR2, and CDR3. The constant region determines the mechanism used to destroy antigen. Antibodies are divided into five major classes, IgM, IgG, IgA, IgD, and IgE, based on their constant region structure and immune function.

In some embodiments, an antibody specifically includes, e.g., intact antibodies (e.g., intact immunoglobulins, e.g., human IgG (e.g., human IgG1, human IgG2, human IgG3, human IgG4)) and antigen-binding antibody fragments. In some embodiments, the antibody is an humanized IgG1 antibody. One example of an antigen-binding domain is an antigen-binding domain formed by a VH-VL dimer. Additional examples of an antibody are described herein. Additional examples of an antibody are known in the art.

As used herein, the term “antigen-binding domain,” or “antigen-binding fragment” is one or more protein domain(s) (e.g., formed from amino acids from a single polypeptide or formed from amino acids from two or more polypeptides (e.g., the same or different polypeptides)) that is capable of specifically binding to one or more different antigen(s). In some examples, an antigen-binding domain can bind to an antigen or epitope with specificity and affinity similar to that of naturally-occurring antibodies. In some embodiments, an antigen-binding domain can include an alternative scaffold. Non-limiting examples of antigen-binding domains are described herein. Additional examples of antigen-binding domains are known in the art. In some examples, an antigen-binding domain can bind to a single antigen. In some embodiments, the antibody, or antigen-binding fragments used in the methods described herein specifically binds to a B cell maturation antigen (BCMA).

An antibody or antigen-binding fragment thereof described herein can be a single polypeptide, or can comprise two, three, four, five, six, seven, eight, nine, or ten (the same or different) polypeptides. In some embodiments where the antibody or antigen-binding fragment thereof is a single polypeptide, the antibody or antigen-binding fragment can comprise a single antigen-binding domain or two antigen-binding domains. In some embodiments where the antibody or antigen-binding fragment is a single polypeptide and comprises two antigen-binding domains, the first and second antigen-binding domains can be identical or different from each other (and can specifically bind to the same or different antigens or epitopes).

In some embodiments where the antibody or the antigen-binding fragment is a single polypeptide, the first antigen-binding domain and the second antigen-binding domain (if present) can each be independently selected from the group of: a VH domain, a VHH domain, a VNAR domain, and a scFv. In some embodiments where the antibody or the antigen-binding fragment is a single polypeptide, the antibody or antigen-binding fragment can be a BiTe, a (scFv)₂, a nanobody, a nanobody-HSA, a DART, a TandAb, a scDiabody, a scDiabody-CH3, scFv-CH-CL-scFv, a HSAbody, scDiabody-HAS, a tandem-scFv, an Adnectin, a DARPin, a fibronectin, and a DEP conjugate. Additional examples of antigen-binding domains that can be used when the antibody or antigen-binding fragment is a single polypeptide are known in the art.

A VHH domain is a single monomeric variable antibody domain that can be found in camelids. A V_{NAR} domain is a single monomeric variable antibody domain that can be found in cartilaginous fish. Non-limiting aspects of VHH domains and V_{NAR} domains are described in, e.g., Cromie et al., Curr. Top. Med. Chem. 15:2543-2557, 2016; De Genst et al., Dev. Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther. 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001; Muyldermans et al., Trends Biochem. Sci. 26:230-235, 2001; Muyldermans, Ann. Rev. Biochem. 82:775-797, 2013; Rahbarizadeh et al., Immunol. Invest. 40:299-338, 2011; Van Audenhove et al., EBioMedicine 8:40-48, 2016; Van Bockstaele et al., Curr Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

In some embodiments where the antibody or antigen-binding fragment is a single polypeptide and comprises two antigen-binding domains, the first antigen-binding domain and the second antigen-binding domain can both be VHH domains, or at least one antigen-binding domain can be a VHH domain. In some embodiments where the antibody or antigen-binding fragment is a single polypeptide and comprises two antigen-binding domains, the first antigen-binding domain and the second antigen-binding domain are both V_{NAR} domains, or at least one antigen-binding domain is a V_{NAR} domain. In some embodiments where the antibody or antigen-binding domain is a single polypeptide, the first antigen-binding domain is a scFv domain. In some embodiments where the antibody or antigen-binding fragment is a single polypeptide and comprises two antigen-binding domains, the first antigen-binding domain and the second antigen-binding domain can both be scFv domains, or at least one antigen-binding domain can be a scFv domain.

In some embodiments, the antibody or antigen-binding fragment can comprise two or more polypeptides (e.g., two, three, four, five, six, seven, eight, nine, or ten polypeptides). In some embodiments where the antibody or antigen-binding fragment comprises two or more polypeptides, two, three, four, five or six of the polypeptides of the two or more polypeptides can be identical.

In some embodiments where the antibody or antigen-binding fragment comprises two or more polypeptides (e.g., two, three, four, five, six, seven, eight, nine, or ten polypeptides), two or more of the polypeptides of the antibody or antigen-binding fragment can assemble (e.g., non-covalently assemble) to form one or more antigen-binding domains, e.g., an antigen-binding fragment of an antibody (e.g., any of the antigen-binding fragments of an antibody described herein), a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv₂, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, and a DEP conjugate. See, e.g., Spiess et al., Mol. Immunol. 67:95-106, 2015, incorporated in its entirety herewith, for a description of these elements. Non-limiting examples of an antigen-binding fragment of an antibody include an Fv fragment, a Fab fragment, a F(ab′)₂ fragment, and a Fab′ fragment. Additional examples of an antigen-binding fragment of an antibody is an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM).

A “Fv” fragment comprises a non-covalently-linked dimer of one heavy chain variable domain and one light chain variable domain.

A “Fab” fragment comprises the constant domain of the light chain and the first constant domain (C_{H1}) of the heavy chain, in addition to the heavy and light chain variable domains of the Fv fragment.

A “F(ab′)₂” fragment comprises two Fab fragments joined, near the hinge region, by disulfide bonds.

A “dual variable domain immunoglobulin” or “DVD-Ig” refers to multivalent and multispecific binding proteins as described, e.g., in DiGiammarino et al., Methods Mol. Biol. 899:145-156, 2012; Jakob et al., MABs 5:358-363, 2013; and U.S. Pat. Nos. 7,612,181; 8,258,268; 8,586,714; 8,716,450; 8,722,855; 8,735,546; and 8,822,645, each of which is incorporated by reference in its entirety.

DARTs are described in, e.g., Garber, Nature Reviews Drug Discovery 13:799-801, 2014.

Afucosylated, or non-fucosylated, monoclonal antibodies are monoclonal antibodies engineered so that the oligosaccharides in the Fc region of the antibody do not have any fucose sugar units. In some embodiments, afucosylation of antibodies increases effects such as antibody-dependent cellular cytotoxicity (ADCC). As described in greater detail below, in some embodiments, the antibodies used in the methods described herein are afucosylated antibodies.

In some embodiments, an antibody described herein can be an IgG1 (e.g., human or humanized IgG1), IgG2 (e.g., human or humanized IgG2), IgG3 (e.g., human or humanized IgG3), IgG4 (e.g., human or humanized IgG4), IgA1 (e.g., human or humanized IgA1), IgA2 (e.g., human or humanized IgA2), IgD (e.g., human or humanized IgD), IgE (e.g., human or humanized IgE), or IgM (e.g., human or humanized IgM).

A humanized antibody is a genetically engineered antibody in which CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557). The acceptor antibody sequences can be, for example, a mature human antibody sequence, a composite of such sequences, a consensus sequence of human antibody sequences, or a germline region sequence. For humanization, an exemplary acceptor sequence for the heavy chain is the germline VH exon VH1-2 and for the J exon (JH), exon JH-3. For the light chain, an exemplary acceptor sequence is exon VL1-12 and J exon JK5.

Thus, a humanized antibody is an antibody having at least four CDRs entirely or substantially from a non-human donor antibody and variable region framework sequences and constant regions, if present, entirely or substantially from human antibody sequences. Similarly a humanized heavy chain has at least two and usually all three CDRs entirely or substantially from a donor antibody heavy chain, and a heavy chain variable region framework sequence and heavy chain constant region, if present, substantially from human heavy chain variable region framework and constant region sequences. Similarly a humanized light chain has at least two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized or human antibody is substantially from or substantially identical to a corresponding CDR in a non-human antibody when at least 60%, 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 70%, 80%, 85%, 90%, 95% or 100% of corresponding residues defined by Kabat are identical.

Although humanized antibodies often incorporate all six CDRs (as defined by Kabat) from a mouse antibody, they can also be made with less than all CDRs (e.g., at least 4 or 5) CDRs from a mouse antibody (e.g., Pascalis et al., J. Immunol. 169:3076, 2002; Vajdos et al., J. Mol. Biol. 320:415-428, 2002; Iwahashi et al., Mol. Immunol. 36:1079-1091, 1999; Tamura et al., J. Immunol. 164:1432-1441, 2000).

Certain amino acids from the human variable region framework residues can be selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid can be substituted by the equivalent framework amino acid from the mouse antibody when it is reasonably expected that the amino acid:
(1) noncovalently binds antigen directly,
(2) is adjacent to a CDR region,
(3) otherwise interacts with a CDR region (e.g. is within about 6 Å of a CDR region); or
(4) mediates interaction between the heavy and light chains.

In some embodiments of any of the antibodies or antigen-binding fragments described herein, the antibody or antigen-binding fragment can comprise a heavy chain variable region comprising a CDR1 comprising DYYIH (SEQ ID NO: 1), a CDR2 comprising YINPNSGYTNYAQKFQG (SEQ ID NO: 2), and a CDR3 comprising YMWERVTGFFDF (SEQ ID NO: 3), and a light chain variable region comprising a CDR1 comprising LASEDISDDLA (SEQ ID NO: 5), a CDR2 comprising TTSSLQS (SEQ ID NO: 6), and a CDR3 comprising QQTYKFPPT (SEQ ID NO: 7).

In some embodiments of any of the antibodies or antigen-binding fragments described herein, the antibody or antigen-binding fragment can comprise a heavy chain variable region comprising a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 4, and/or a light chain variable domain comprising a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8.

In some embodiments of any of the antibodies or antigen-binding fragments described herein, the antibody or antigen-binding fragment can comprise a heavy chain variable region encoded by a nucleic acid comprising a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 11, and/or a light chain variable domain encoded by a nucleic acid comprising a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 12.

Exemplary Heavy Chain Variable Domain
(SEQ ID NO: 4)
QVQLVQSGAEVKKPGASVKLSCKASGYTFTDYYIHWVRQAPGQGLEWIGYINPNSGYT
NYAQKFQGRATMTADKSINTAYVELSRLRSDDTAVYFCTRYMWERVTGFFDFWGQGT
MVTVSS
DNA Encoding Exemplary Heavy Chain Variable Domain
(SEQ ID NO: 11)
caagtgcagc tggtgcagtc cggagcggaa gtgaagaaac ctggggcgtc
cgtgaagctc agctgcaagg cctccggcta cactttcacc gattactaca
tccactgggt cagacaggca ccgggacagg gactggagtg gattggttac
atcaacccca actccgggta caccaattac gcccagaagt tccagggtcg
ggctacgatg accgccgaca agtcgatcaa cactgcctac gtggaactgt
caaggctgcg gtccgatgac accgccgtgt acttctgtac ccgctatatg
tgggagcgcg tgactggatt tttcgacttc tggggccaag gcaccatggt
caccgtgtcg agc
Exemplary Light Chain Variable Domain
(SEQ ID NO: 8)
DIQMTQSPSSVSASVGDRVTITCLASEDISDDLAWYQQKPGKAPKVLVYTTSSLQSGVPS
RFSGSGSGTDFTLTISSLQPEDFATYFCQQTYKFPPTFGGGTKVEIKR
DNA Encoding Exemplary Light Chain Variable Domain
(SEQ ID NO: 12)
gacattcaga tgacccagtc cccctcgtcc gtgtccgctt ccgtgggaga
tcgcgtgacc atcacttgtc ttgcgtccga ggatatctca gacgacctgg
cctggtacca gcagaagcct ggaaaggccc cgaaggtcct ggtgtacact
accagcagcc tccagtcggg cgtgccttca cggttctccg gttcggggtc
tggcaccgac ttcaccctga ctattagctc cctgcaaccc gaggacttcg
ccacctactt ttgccagcaa acctacaagt tcccgccaac gttcggaggg
ggcaccaagg tcgaaatcaa acgt

In some embodiments of any of the antibodies or antigen-binding fragments described herein, the antibody or antigen-binding fragment can comprise a heavy chain comprising a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 13, and/or a light chain comprising a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 15.

In some embodiments of any of the antibodies or antigen-binding fragments described herein, the antibody or antigen-binding fragment can comprise a heavy chain encoded by a nucleic acid comprising a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 14, and/or a light chain encoded by a nucleic acid comprising a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 16.

Exemplary Heavy Chain
(SEQ ID NO: 13)
QVQLVQSGAEVKKPGASVKLSCKASGYTFTDYYIHWVRQAPGQGLEWIGYINPNSGYT
NYAQKFQGRATMTADKSINTAYVELSRLRSDDTAVYFCTRYMWERVTGFFDFWGQGT
MVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPPCP
APELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK
DNA Encoding Exemplary Heavy Chain
(SEQ ID NO: 14)
caagtgcagc tggtgcagtc cggagcggaa gtgaagaaac ctggggcgtc
cgtgaagctc agctgcaagg cctccggcta cactttcacc gattactaca
tccactgggt cagacaggca ccgggacagg gactggagtg gattggttac
atcaacccca actccgggta caccaattac gcccagaagt tccagggtcg
ggctacgatg accgccgaca agtcgatcaa cactgcctac gtggaactgt
caaggctgcg gtccgatgac accgccgtgt acttctgtac ccgctatatg
tgggagcgcg tgactggatt tttcgacttc tggggccaag gcaccatggt
caccgtgtcg agcgctagca ccaagggccc atcggtcttc cccctggcac
cctcctccaa gagcacctct gggggcacag cggccctggg ctgcctggtc
aaggactact tccccgaacc ggtgacggtg tcgtggaact caggcgccct
gaccagcggc gtgcacacct tcccggccgt cctacagtcc tcaggactct
actccctcag cagcgtggtg accgtgccct ccagcagctt gggcacccag
acctacatct gcaacgtgaa tcacaagccc agcaacacca aggtggacaa
gaaggttgag cccaaatctt gtgacaaaac tcacacatgc ccaccgtgcc
cagcacctga actcctgggg ggaccgtcag tcttcctctt ccccccaaaa
cccaaggaca ccctcatgat ctcccggacc cctgaggtca catgcgtggt
ggtggacgtg agccacgaag accctgaggt caagttcaac tggtacgtgg
acggcgtgga ggtgcataat gccaagacaa agccgcggga ggagcagtac
aacagcacgt accgtgtggt cagcgtcctc accgtcctgc accaggactg
gctgaatggc aaggagtaca agtgcaaggt ctccaacaaa gccctcccag
cccccatcga gaaaaccatc tccaaagcca aagggcagcc ccgagaacca
caggtgtaca ccctgccccc atcccgggac gagctgacca agaaccaggt
cagcctgacc tgcctggtca aaggcttcta tcccagcgac atcgccgtgg
agtgggagag caatgggcag ccggagaaca actacaagac cacgcctccc
gtgctggact ccgacggctc cttcttcctc tacagcaagc tcaccgtgga
caagagcagg tggcagcagg ggaacgtctt ctcatgctcc gtgatgcatg
aggctctgca caaccactac acgcagaaga gcctctccct gtctccgggt aaa
Exemplary Light Chain
(SEQ ID NO: 15)
DIQMTQSPSSVSASVGDRVTITCLASEDISDDLAWYQQKPGKAPKVLVYTTSSLQSGVPS
RFSGSGSGTDFTLTISSLQPEDFATYFCQQTYKFPPTFGGGTKVEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC
DNA Encoding Exemplary Light Chain
(SEQ ID NO: 16)
gacattcaga tgacccagtc cccctcgtcc gtgtccgctt ccgtgggaga
tcgcgtgacc atcacttgtc ttgcgtccga ggatatctca gacgacctgg
cctggtacca gcagaagcct ggaaaggccc cgaaggtcct ggtgtacact
accagcagcc tccagtcggg cgtgccttca cggttctccg gttcggggtc
tggcaccgac ttcaccctga ctattagctc cctgcaaccc gaggacttcg
ccacctactt ttgccagcaa acctacaagt tcccgccaac gttcggaggg
ggcaccaagg tcgaaatcaa acgtacggtg gctgcaccat ctgtcttcat
cttcccgcca tctgatgagc agttgaaatc tggaactgcc tctgttgtgt
gcctgctgaa taacttctat cccagagagg ccaaagtaca gtggaaggtg
gataacgccc tccaatcggg taactcccag gagagtgtca cagagcagga
cagcaaggac agcacctaca gcctcagcag caccctgacg ctgagcaaag
cagactacga gaaacacaaa gtctacgcct gcgaagtcac ccatcagggc
ctgagctcgc ccgtcacaaa gagcttcaac aggggagagt gt

In some embodiments of any of the antibodies or antigen-binding fragments described herein, the antibody is one as described in US 2017/0233484 (see also WO 2017/143069). In one such embodiment, the antibody or antigen-binding fragment includes the hSG16.17 VH3 antibody, which comprises a heavy chain variable region comprising a CDR1, CDR2 and CDR3 corresponding to SEQ ID NOs: 60-62 respectively, as listed in US 2017/0233484 and WO 2017/143069, and a light chain variable domain comprising CDR1, CDR2 and CDR3 corresponding to SEQ ID NOs: 90-92, respectively, as listed in US 2017/0233484 and WO 2017/143069. The VH and VL domains of hSG16.17 VH3 correspond to SEQ ID NOs: 13 and 19, respectively, as listed in US 2017/0233484 and WO 2017/143069.

Heavy and light chain variable regions of humanized antibodies can be linked to at least a portion of a human constant region. The choice of constant region depends, in part, whether antibody-dependent cell-mediated cytotoxicity, antibody dependent cellular phagocytosis, and/or complement dependent cytotoxicity are desired. For example, human isotopes IgG1 and IgG3 have strong complement-dependent cytotoxicity, human isotype IgG2 weak complement-dependent cytotoxicity, and human IgG4 lacks complement-dependent cytotoxicity. Human IgG1 and IgG3 also induce stronger cell mediated effector functions than human IgG2 and IgG4. Light chain constant regions can be lambda or kappa. Antibodies can be expressed as tetramers containing two light and two heavy chains, as separate heavy chains, light chains, as Fab, Fab′, F(ab′)₂, and Fv, or as single chain antibodies in which heavy and light chain variable domains are linked through a spacer.

One or several amino acids at the amino or carboxy terminus of the light and/or heavy chain, such as the C-terminal lysine of the heavy chain, may be missing or derivatized in a portion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. U.S.A. 103:4005, 2006), or to prolong half-life in humans (see, e.g., Hinton et al., J. Biol. Chem. 279:6213, 2004).

Exemplary substitutions include a substitution of a native amino acid to a cysteine residue at amino acid position 234, 235, 237, 239, 267, 298, 299, 326, 330, or 332, preferably an S239C mutation in a human IgG1 heavy chain (numbering is according to the EU index (Kabat, Sequences of Proteins of Immunological Interest (National Institutes of Health, Bethesda, Md., 1987 and 1991); see US 20100158909, which is herein incorporated reference). A heavy chain can include a S239C substitution, with and without a C-terminal lysine. The presence of an additional cysteine residue allows interchain disulfide bond formation. Such interchain disulfide bond formation can cause steric hindrance, thereby reducing the affinity of the Fc region-FcγR binding interaction. The cysteine residue(s) introduced in or in proximity to the Fc region of an IgG constant region can also serve as sites for conjugation to therapeutic agents (i.e., coupling cytotoxic drugs using thiol specific reagents such as maleimide derivatives of drugs. The presence of a therapeutic agent causes steric hindrance, thereby further reducing the affinity of the Fc region-FcTR binding interaction. Other substitutions at any of heavy chain amino acid positions 234, 235, 236 and/or 237 reduce affinity for Fcγ receptors, particularly FcγRI receptor (see, e.g., U.S. Pat. Nos. 6,624,821, 5,624,821.) A preferred combination of heavy chain amino acid substitutions is S239D, A330L and 1332E, which increases the affinity of the Fc domain for FcγRIIIA and consequently increases ADCC.

The in vivo half-life of an antibody can also impact its effector functions. The half-life of an antibody can be increased or decreased to modify its therapeutic activities. FcRn is a receptor that is structurally similar to MHC Class I antigen that non-covalently associates with β2-microglobulin. FcRn regulates the catabolism of IgGs and their transcytosis across tissues (Ghetie and Ward, Annu. Rev. Immunol. 18:739-766, 2000; Ghetie and Ward, Immunol. Res. 25:97-113, 2002). The IgG-FcRn interaction takes place at pH 6.0 (pH of intracellular vesicles) but not at pH 7.4 (pH of blood); this interaction enables IgGs to be recycled back to the circulation (Ghetie and Ward, Ann. Rev. Immunol. 18:739-766, 2000; Ghetie and Ward, Immunol. Res. 25:97-113, 2002). The region on human IgG1 involved in FcRn binding has been mapped (Shields et al., J. Biol. Chem. 276:6591-604, 2001). Alanine substitutions at heavy chain amino acid positions Pro238, Thr256, Thr307, Gln311, Asp312, Glu380, Glu382, or Asn434 of human IgG1 enhance FcRn binding (Shields et al., J. Biol. Chem. 276:6591-604, 2001). IgG1 molecules harboring these substitutions have longer serum half-lives. Consequently, these modified IgG1 molecules may be able to carry out their effector functions, and hence exert their therapeutic efficacies, over a longer period of time compared to unmodified IgG1. Other exemplary substitutions in a heavy chain for increasing binding to FcRn include introduction of a Gln at amino acid position 250 and/or a Leu at amino acid position 428. EU numbering is used for all positions in the constant region.

Oligosaccharides covalently attached to the conserved Asn297 are involved in the ability of the Fc region of an IgG to bind FcγR (Lund et al., J. Immunol. 157:4963-69, 1996; Wright and Morrison, Trends Biotechnol. 15:26-31, 1997). Engineering of this glycoform on IgG can significantly improve IgG-mediated ADCC. Addition of bisecting N-acetylglucosamine modifications (Umana et al., Nat. Biotechnol. 17:176-180, 1999; Davies et al., Biotech. Bioeng. 74:288-94, 2001) to this glycoform or removal of fucose (Shields et al., J. Biol. Chem. 277:26733-40, 2002; Shinkawa et al., J. Biol. Chem. 278:6591-604, 2003; Niwa et al., Cancer Res. 64:2127-33, 2004) from this glycoform are two examples of IgG Fc engineering that improves the binding between IgG Fc and FcγR, thereby enhancing Ig-mediated ADCC activity.

A systemic substitution of solvent-exposed amino acids of human IgG1 Fc region has generated IgG variants with altered FcγR binding affinities (Shields et al., J. Biol. Chem. 276:6591-604, 2001). When compared to parental IgG1, a subset of these variants involving substitutions at Thr256/Ser298, Ser298/Glu333, Ser298/Lys334, or Ser298/Glu333/Lys334 to Ala demonstrate increased in both binding affinity toward FcγR and ADCC activity (Shields et al., J. Biol. Chem. 276:6591-604, 2001; Okazaki et al., J. Mol. Biol. 336:1239-49, 2004).

Complement fixation activity of antibodies (both C1q binding and CDC activity) can be improved by substitutions at Lys326 and Glu333 (Idusogie et al., J. Immunol. 166:2571-2575, 2001). The same substitutions on a human IgG2 backbone can convert an antibody isotype that binds poorly to C1q and is severely deficient in complement activation activity to one that can both bind C1q and mediate CDC (Idusogie et al., J. Immunol. 166:2571-75, 2001). Several other methods have also been applied to improve complement fixation activity of antibodies. For example, the grafting of an 18-amino acid carboxyl-terminal tail piece of IgM to the carboxyl-termini of IgG greatly enhances their CDC activity. This is observed even with IgG4, which normally has no detectable CDC activity (Smith et al., J. Immunol. 154:2226-36, 1995). Also, substituting Ser444 located close to the carboxy-terminal of IgG1 heavy chain with Cys induced tail-to-tail dimerization of IgG1 with a 200-fold increase of CDC activity over monomeric IgG1 (Shopes et al., J. Immunol. 148:2918-22, 1992). In addition, a bispecific diabody construct with specificity for C1q also confers CDC activity (Kontermann et al., Nat. Biotech. 15:629-31, 1997).

Complement activity can be reduced by mutating at least one of the amino acid residues 318, 320, and 322 of the heavy chain to a residue having a different side chain, such as Ala. Other alkyl-substituted non-ionic residues, such as Gly, Ile, Leu, or Val, or such aromatic non-polar residues as Phe, Tyr, Trp and Pro in place of any one of the three residues also reduce or abolish C1q binding. Ser, Thr, Cys, and Met can be used at residues 320 and 322, but not 318, to reduce or abolish C1q binding activity. Replacement of the 318 (Glu) residue by a polar residue may modify but not abolish C1q binding activity. Replacing residue 297 (Asn) with Ala results in removal of lytic activity, but only slightly reduces (about three-fold weaker) affinity for C1q. This alteration destroys the glycosylation site and the presence of carbohydrate that is required for complement activation. Any other substitution at this site also destroys the glycosylation site. The following heavy chain substitutions and any combination thereof also reduce C1q binding: D270A, K322A, P329A, and P311S (see WO 06/036291).

Reference to a human constant region includes a constant region with any natural allotype or any permutation of residues occupying polymorphic positions in natural allotypes. Also, up to 1, 2, 5, or 10 mutations may be present relative to a natural human constant region, such as those indicated above to reduce Fcγ receptor binding or increase binding to FcRN.

### Non-Fucosylated Antibodies or Antigen-Binding Fragments

In some embodiments, any of the antibodies or antigen-binding fragments as described herein have reduced fucosylation or are non-fucosylated and can be utilized in the methods that are provided. For example, in some embodiments, the antibody or antigen-binding fragment has reduced core fucosylation. “Core fucosylation” refers to addition of fucose (“fucosylation”) to N-acetylglucosamine (“GlcNAc”) at the reducing terminal of an N-linked glycan.

A “complex N-glycoside-linked sugar chain” is typically bound to asparagine 297 (according to the number of Kabat). As used herein, the complex N-glycoside-linked sugar chain has a biantennary composite sugar chain, mainly having the following structure:

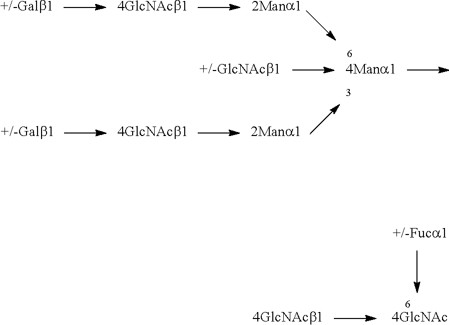

where ± indicates the sugar molecule can be present or absent, and the numbers indicate the position of linkages between the sugar molecules. In the above structure, the sugar chain terminal which binds to asparagine is called a reducing terminal (at right), and the opposite side is called a non-reducing terminal. Fucose is usually bound to N-acetylglucosamine (“GlcNAc”) of the reducing terminal, typically by an α1,6 bond (the 6-position of GlcNAc is linked to the 1-position of fucose). “Gal” refers to galactose, and “Man” refers to mannose.

A “complex N-glycoside-linked sugar chain” includes 1) a complex type, in which the non-reducing terminal side of the core structure has one or more branches of galactose-N-acetylglucosamine (also referred to as “gal-GlcNAc”) and the non-reducing terminal side of Gal-GlcNAc optionally has a sialic acid, bisecting N-acetylglucosamine or the like; or 2) a hybrid type, in which the non-reducing terminal side of the core structure has both branches of a high mannose N-glycoside-linked sugar chain and complex N-glycoside-linked sugar chain. In some embodiments, the “complex N-glycoside-linked sugar chain” includes a complex type in which the non-reducing terminal side of the core structure has zero, one or more branches of galactose-N-acetylglucosamine (also referred to as “gal-GlcNAc”) and the non-reducing terminal side of Gal-GlcNAc optionally further has a structure such as a sialic acid, bisecting N-acetylglucosamine or the like.

In certain embodiments, typically only a minor amount of fucose is incorporated into the complex N-glycoside-linked sugar chain(s) of the antibodies or antigen-binding fragments disclosed herein. For example, in various embodiments, less than about 60%, less than about 50%, less than about 40%, less than about 30%, less than about 20%, less than about 15%, less than about 10%, less than about 5%, or less than about 3% of the molecules of an antibody have core fucosylation by fucose. In some embodiments, about 2% of the molecules of the antibody has core fucosylation by fucose.

In some embodiments, only a minor amount of a fucose analog (or a metabolite or product of the fucose analog) is incorporated into the complex N-glycoside-linked sugar chain(s). For example, in various embodiments, less than about 60%, less than about 50%, less than about 40%, less than about 30%, less than about 20%, less than about 15%, less than about 10%, less than about 5%, or less than about 3% of the antibodies or antigen-binding fragment have core fucosylation by a fucose analog or a metabolite or product of the fucose analog. In some embodiments, about 2% of the antibody or antigen-binding fragment have core fucosylation by a fucose analog or a metabolite or product of the fucose analog.

In some of any of the embodiments disclosed herein, the antibody is an afucosylated antibody, meaning that the antibody at position N297 (EU numbering) does not contain fucose or that a population of such antibodies collectively have no fucose at this position or only have a very low level of fucosylation. For example, in certain embodiments, the antibodies are >90%, or are >95% afucosylated. In some embodiments, the antibodies are at least 95-98% afucosylated, or at least 98-99% afucosylated.

Methods of making non-fucosylated antibodies by incubating antibody-producing cells with a fucose analogue are described, e.g., in WO2009/135181. Briefly, cells that have been engineered to express an antibody or antigen-binding fragment are incubated in the presence of a fucose analogue or an intracellular metabolite or product of the fucose analog. An intracellular metabolite can be, for example, a GDP-modified analog or a fully or partially de-esterified analog. A product can be, for example, a fully or partially de-esterified analog. In some embodiments, a fucose analogue can inhibit an enzyme(s) in the fucose salvage pathway. For example, a fucose analog (or an intracellular metabolite or product of the fucose analog) can inhibit the activity of fucokinase, or GDP-fucose-pyrophosphorylase. In some embodiments, a fucose analog (or an intracellular metabolite or product of the fucose analog) inhibits fucosyltransferase (preferably a 1,6-fucosyltransferase, e.g., the FUT8 protein). In some embodiments, a fucose analog (or an intracellular metabolite or product of the fucose analog) can inhibit the activity of an enzyme in the de novo synthetic pathway for fucose. For example, a fucose analog (or an intracellular metabolite or product of the fucose analog) can inhibit the activity of GDP-mannose 4,6-dehydratase or/or GDP-fucose synthetase. In some embodiments, the fucose analog (or an intracellular metabolite or product of the fucose analog) can inhibit a fucose transporter (e.g., GDP-fucose transporter).

In certain embodiments, the fucose analogue is 2-flurofucose. Methods of using fucose analogues in growth medium and other fucose analogues are disclosed, e.g., in WO/2009/135181.

Other methods for engineering cell lines to reduce core fucosylation included gene knock-outs, gene knock-ins and RNA interference (RNAi). In gene knock-outs, the gene encoding FUT8 (alpha 1,6-fucosyltransferase enzyme) is inactivated. FUT8 catalyzes the transfer of a fucosyl residue from GDP-fucose to position 6 of Asn-linked (N-linked) GlcNac of an N-glycan. FUT8 is reported to be the only enzyme responsible for adding fucose to the N-linked biantennary carbohydrate at Asn297. Gene knock-ins add genes encoding enzymes such as GNTIII or a golgi alpha mannosidase II. An increase in the levels of such enzymes in cells diverts monoclonal antibodies from the fucosylation pathway (leading to decreased core fucosylation), and having increased amount of bisecting N-acetylglucosamines. RNAi typically also targets FUT8 gene expression, leading to decreased mRNA transcript levels or knocking out gene expression entirely. Any of these methods can be used to generate a cell line that would be able to produce a non-fucosylated antibody.

Many methods are available to determine the amount of fucosylation on an antibody. Methods include, e.g., LC-MS via PLRP-S chromatography and electrospray ionization quadrupole TOF MS.

### Production of Antibodies and Antigen-Binding Fragments

Antibodies and antigen-binding fragments are typically produced by recombinant expression. Recombinant polynucleotide constructs typically include an expression control sequence operably linked to the coding sequences of antibody chains, including naturally-associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the produced antibodies or antigen-binding fragments.

Mammalian cells are a preferred host for expressing nucleotide segments encoding antibodies and antigen-binding fragments. See Winnacker, From Genes to Clones, (VCH Publishers, N Y, 1987). A number of suitable host cell lines capable of secreting intact heterologous proteins have been developed in the art, and include CHO cell lines (e.g., DG44), various COS cell lines, HeLa cells, HEK293 cells, L cells, and non-antibody-producing myelomas including Sp2/0 and NS0. Preferably, the cells are nonhuman. Expression vectors for these cells can include expression control sequences, such as an origin of replication, a promoter, an enhancer (Queen et al., Immunol. Rev. 89:49, 1986), and necessary processing information sites, such as ribosome binding sites, RNA splice sites, polyadenylation sites, and transcriptional terminator sequences. Preferred expression control sequences are promoters derived from endogenous genes, cytomegalovirus, SV40, adenovirus, bovine papillomavirus, and the like. See Co et al., J. Immunol. 148:1149, 1992.

Once expressed, antibodies and antigen-binding fragments can be purified according to standard procedures of the art, including HPLC purification, column chromatography, gel electrophoresis and the like (see generally, Scopes, Protein Purification (Springer-Verlag, NY, 1982)).

### Nirogacestat

Nirogacestat is a selective, reversible, noncompetitive inhibitor of γ-secretase. In some embodiments of any of the methods described herein, nirogacestat ((S)-2-(((S)-6,8-difluoro-1,2,3,4-tetrahydronaphthalen-2-yl)amino)-N-(1-(2-methyl-1-(neopentylamino)propan-2-yl)-1H-imidazol-4-yl)pentanamide), (PF-03084014), has the structure of Compound I:

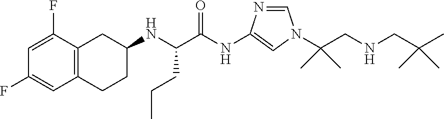 [SMILES: CCC[C@H](N[C@H]1CCc2cc(F)cc(F)c2C1)C(=O)Nc1cn(C(C)(C)CNCC(C)(C)C)cn1]

or a pharmaceutically acceptable salt thereof. In some embodiments, the pharmaceutically acceptable salt is hydrobromide (e.g., nirogacestat hydrobromide). In other embodiments, the pharmaceutically acceptable salt is dihydrobromide (e.g., nirogacestat dihydrobromide). Known carriers can be include in the formulation for oral administration of nirogacestat, e.g., microcrystalline cellulose, sodium citrate, calcium carbonate, dicalcium phosphate and glycine, along with disintegrants (e.g., starch (e.g., corn, potato, or tapioca starch)), methylcellulose, alginic acid, and certain complex silicates, granulation binders (e.g., polyvinylpyroolidone, sucrose, gelatin, and acacia), lubricating agents (e.g., magnesium stearate, sodium lauryl sulfate and talc). In some embodiments, nirogacestat is combined with various sweetening and/or flavoring agents, coloring dyes.

### Pharmaceutical Compositions

The pharmaceutical compositions used in any of the methods described herein include an antibody, or antigen-binding fragment thereof, that specifically binds to a B cell maturation antigen (BCMA) (e.g., any of the exemplary antibodies or antigen-binding fragments described herein) and/or dexamethasone. Other pharmaceutical compositions used in any of the methods described herein include nirogacestat.

Pharmaceutical compositions comprising an antibody or antigen-binding fragment and/or dexamethasone can be formulated for systemic (e.g., intravenous) administration. Pharmaceutical compositions comprising nirogacestat can be formulated for oral administration.

Methods of generating pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral (e.g., intravenous), intradermal, or subcutaneous application can include the following components: a sterile diluent, such as water for injection, saline solution, fixed oils, polyethylene glycols, glycerine, propylene glycol or other synthetic solvents; antibacterial agents, such as benzyl alcohol or methyl parabens; antioxidants, such as ascorbic acid or sodium bisulfite; chelating agents, such as ethylenediaminetetraacetic acid; buffers, such as acetates, citrates, or phosphates; and agents for the adjustment of tonicity, such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ), or phosphate buffered saline (PBS). In some embodiments, the pharmaceutically acceptable carrier is a sodium chloride solution. In all cases, the composition should be sterile. The compositions should be stable under the conditions of manufacture and storage, and must be preserved against the contaminating action of microorganisms, such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating, such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, the composition can include isotonic agents, for example, sugars, polyalcohols, such as mannitol, sorbitol, and sodium chloride in the composition. Prolonged absorption of the injectable compositions can be achieved by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the methods of preparation can include the use of vacuum drying and freeze-drying, which yield a powder of the active ingredient, plus any additional desired ingredient from a previously sterile-filtered solution thereof.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

The one or more doses of the pharmaceutical composition comprising nirogacestat ((S)-2-(((S)-6,8-difluoro-1,2,3,4-tetrahydronaphthalen-2-yl)amino)-N-(1-(2-methyl-1-(neopentylamino)propan-2-yl)-1H-imidazol-4-yl)pentanamide), (PF-03084014), can be formulated for oral administration (e.g., any of the pharmaceutically acceptable salt forms of nirogacestat described herein or known in the art, e.g., nirogacestat hydrobromide or nirogacestat dihydrobromide). In some embodiments, the one or more doses of the pharmaceutical composition comprising nirogacestat or a pharmaceutically acceptable salt thereof is formulated as a tablet, capsule, or aqueous suspension. Non-limiting examples of carriers that can be present in a pharmaceutical composition comprising nirogacestat include microcrystalline cellulose, sodium citrate, calcium carbonate, dicalcium phosphate, and glycine. Non-limiting examples of disintegrants that can be present in a pharmaceutical composition comprising nirogacestat include starch (preferably corn, potato, or tapioca starch), methylcellulose, alginic acid, and certain complex silicates. Non-limiting examples of granulation binders that can be present in a pharmaceutical composition comprising nirogacestat include polyvinylpyrrolidone, sucrose, gelatin, and acacia. Lubricating agents such as magnesium stearate, sodium lauryl sulfate and talc are often useful for tableting purposes. Solid compositions of a similar type may also be employed as fillers in gelatin capsules. Preferred materials in this connection include lactose or milk sugar as well as high molecular weight polyethylene glycols. When aqueous suspensions and/or elixers are desired for oral administration, the active ingredient may be combined with various sweetening or flavoring agents, coloring matter or dyes, and, if so desired, emulsifying and/or suspending agents as well, together with such diluents as water, ethanol, glycerin, and various like combinations thereof.

### Methods of Treatment

Provided herein are methods of treating a subject having multiple myeloma (MM) that include administering to the subject one or more doses of an antibody, or antigen binding fragment thereof, that specifically binds to a B cell maturation antigen (BCMA) (e.g., any of the exemplary antibodies or antigen-binding fragments described herein) and one or more doses of nirogacestat (e.g., nirogacestat dihydrobromide or nirogacestat hydrobromide).

Also provided herein are methods of treating a subject having multiple myeloma (MM) that include administering to the subject one or more doses of an antibody, or antigen binding fragment thereof, that specifically binds to a B cell maturation antigen (BCMA) (e.g., any of the exemplary antibodies or antigen-binding fragments described herein), one or more doses of nirogacestat (e.g., nirogacestat dihydrobromide, nirogacestat hydrobromide), and one or more doses of dexamethasone.

As used herein, a “subject” typically refers to a human subject, such as a human patient that has multiple myeloma (MM). In some embodiments, the subject has been identified or diagnosed as having a precursor to myeloma, a multiple myeloma cancer which produces light chains of kappa-type and/or light chains of lambda-type, aggressive multiple myeloma, refractory multiple myeloma, or drug-resistant multiple myeloma. In some embodiments, the subject has been identified or diagnosed as having relapsed or refractory multiple myeloma (RRMM). Diagnosis of MM requiring systemic therapy is defined by International Myeloma Working Group (IMWG) 2014 criteria (Rajkumar, et al. (2014) Lancet Oncol, 15(12):e538-48).

In some embodiments, the subject is evaluated to determine if the subject has a small nucleotide polymorphism of FcγRII and/or FcγRIII. In some embodiments, the small nucleotide polymorphisms of FcγRII and FcγRIII may be determined by, for example, testing of the polymorphisms of FCGRIIIA-158V/F, and/or FCGRIIA-131H/R. Accordingly, in some embodiments, the subject has a small nucleotide polymorphism of FcγRII and/or FcγRIII.

In some embodiments, the subject was previously administered one or more therapeutic agents or treatments for multiple myeloma. The one or more previously administered therapeutic agents or treatments for multiple myeloma include, but are not limited to, a proteasome inhibitor (PI), an immunomodulatory drug (IMiD), and an anti-CD38 antibody. In some embodiments, the one or more (e.g., one, two, or three) previously administered therapeutic agents or treatments (e.g., one or more of PIs, IMiDs, and anti-CD38 antibodies) were not effective in treating the multiple myeloma in the subject. In some embodiments, the subject has previously been administered a BCMA-directed myeloma therapy other than at least one of a proteasome inhibitor, an immunomodulatory agent, and an anti-CD38 antibody, or cannot tolerate any of the foregoing. In some embodiments, the subject was previously administered at least one BCMA-directed myeloma therapy selected from the group consisting of: ADC, CAR-T cell therapy, and bispecific antibodies targeted to human BCMA.

In some embodiments, the subject has one or more of: a serum monoclonal paraprotein (M-protein) level of ≥0.5 g/dL, a urine M-protein level of ≥200 mg/24 hours, a serum immunoglobulin free light chain level of ≥10 mg/dL, and/or an abnormal serum immunoglobulin kappa to lambda free light chain ratio.

In some embodiments, the cancer cells in the subject having MM show detectable levels of BCMA measured at either the protein (e.g., by immunoassay using one of the exemplified antibodies) or mRNA level. In some embodiments, the cancer cells in the subject having MM show elevated levels of BCMA relative to noncancerous tissue of the same type, e.g., from the same or a similar patient. An exemplary level of BCMA on cancer cells can be 5000-150000 BCMA molecules per cell. Optionally, a level of BCMA in a cancer cell from a subject can be measured before administering treatment. In some embodiments, the methods described herein can further include a step of selecting a subject having a multiple myeloma. In some embodiments, specific criteria are applied to the selection of subjects (e.g., any of the inclusion criteria described herein). Such criteria include characteristics of the subjects such as age, gender, the type and stage of a disease, previous treatment history, and other medical conditions. In some embodiments, the methods described herein can further include terminating the treatment due to the condition of the subject (e.g., using any of the termination criteria described herein).

A. Combination Therapy with Nirogacestat

Provided herein are methods of treating a subject having multiple myeloma (MM) that include administering to the subject one or more doses of an antibody, or antigen binding fragment thereof, that specifically binds to a B cell maturation antigen (BCMA) (e.g., any of the exemplary antibodies or antigen-binding fragments described herein) and one or more doses of nirogacestat (e.g., nirogacestat dihydrobromide or nirogacestat hydrobromide).

In some embodiments, the antibody or antigen-binding fragment thereof is a non-fucosylated antibody or antigen-binding fragment thereof. In some embodiments, the antibody or antigen-binding fragment thereof is administered to the subject, and wherein about or at least 95%, 97%, 98% or 99% of the antibody or antigen-binding fragment thereof in the composition are afucosylated. In some embodiments, the antibody or antigen-binding fragment thereof, comprises: a heavy chain variable region comprising a CDR1 comprising SEQ ID NO: 1, a CDR2 comprising SEQ ID NO: 2, and a CDR3 comprising SEQ ID NO: 3, and a light chain variable domain comprising a CDR1 comprising SEQ ID NO: 5, a CDR2 comprising SEQ ID NO: 6, and a CDR3 comprising SEQ ID NO: 7.

In some embodiments of any of the methods described herein, the antibody or the antigen-binding fragment thereof comprises a heavy chain variable domain comprising an amino acid sequence that is at least 80% identical to SEQ ID NO: 4 and a light chain variable domain comprising an amino acid sequence that is at least 80% identical to SEQ ID NO: 8.

In some embodiments, the antibody or the antigen-binding fragment thereof comprises a heavy chain variable domain comprising an amino acid sequence that is at least 90% identical to SEQ ID NO: 4 and a light chain variable domain comprising an amino acid sequence that is at least 90% identical to SEQ ID NO: 8. In some embodiments, the antibody or the antigen-binding fragment thereof comprises a heavy chain variable domain comprising an amino acid sequence of SEQ ID NO: 4 and a light chain variable domain comprising an amino acid sequence of SEQ ID NO: 8.

In some embodiments of any of the methods described herein, the antibody or the antigen-binding fragment thereof is humanized. In some embodiments of any of the methods described herein, the antibody is an IgG1 antibody. In some embodiments of any of the methods described herein, the antibody or antigen-binding fragment thereof is not a bispecific antibody, a bispecific T cell engager (BiTE), a chimeric antigen receptor (CAR), or an antibody drug conjugate (ADC), or a portion thereof.

### General Dosing of BCMA Antibody or Antigen-Fragment Thereof And Nirogacestat

In some embodiments of any of the methods described herein, the one or more doses of the antibody or the antigen-binding fragment thereof are independently administered to the subject at about 100 mg of the antibody or antigen-binding fragment thereof to about 2000 mg of the antibody or the antigen-binding fragment thereof (e.g., about 100 mg to about 1800 mg, about 100 mg to about 1600 mg, about 100 mg to about 1400 mg, about 100 mg to about 1200 mg, about 100 mg to about 1000 mg, about 100 mg to about 800 mg, about 100 mg to about 600 mg, about 100 mg to about 400 mg, about 100 mg to about 200 mg, about 200 mg to about 2000 mg, about 200 mg to about 1800 mg, about 200 mg to about 1600 mg, about 200 mg to about 1400 mg, about 200 mg to about 1200 mg, about 200 mg to about 1000 mg, about 200 mg to about 800 mg, about 200 mg to about 600 mg, about 200 mg to about 400 mg, about 400 mg to about 2000 mg, about 400 mg to about 1800 mg, about 400 mg to about 1600 mg, about 400 mg to about 1400 mg, about 400 mg to about 1200 mg, about 400 mg to about 1000 mg, about 400 mg to about 800 mg, about 400 mg to about 600 mg, about 600 mg to about 2000 mg, about 600 mg to about 1800 mg, about 600 mg to about 1600 mg, about 600 mg to about 1400 mg, about 600 mg to about 1200 mg, about 600 mg to about 1000 mg, about 600 mg to about 800 mg, about 800 mg to about 2000 mg, about 800 mg to about 1800 mg, about 800 mg to about 1600 mg, about 800 mg to about 1400 mg, about 800 mg to about 1200 mg, about 800 mg to about 1000 mg, about 1000 mg to about 2000 mg, about 1000 mg to about 1800 mg, about 1000 mg to about 1600 mg, about 1000 mg to about 1400 mg, about 1000 mg to about 1200 mg, about 1200 mg to about 2000 mg, about 1200 mg to about 1800 mg, about 1200 mg to about 1600 mg, about 1200 mg to about 1400 mg, about 1400 mg to about 2000 mg, about 1400 mg to about 1800 mg, about 1400 mg to about 1600 mg, about 1600 mg to about 2000 mg, about 1600 mg to about 1800 mg, about 1800 mg to about 2000 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg).

In some embodiments, the one or more doses of the antibody or antigen-binding fragment are independently administered to the subject at about 100 mg of the antibody or antigen-binding fragment to about 2,000 mg of the antibody or antigen-binding fragment (e.g., about 100 mg to about 1,950 mg, about 100 mg to about 1,900 mg, about 100 mg to about 1,850 mg, about 100 mg to about 1,800 mg, about 100 mg to about 1,750 mg, about 100 mg to about 1,700 mg, about 100 mg to about 1,650 mg, about 100 mg to about 1,600 mg, about 100 mg to about 1,550 mg, about 100 mg to about 1,500 mg, about 100 mg to about 1,450 mg, about 100 mg to about 1,400 mg, about 100 mg to about 1,350 mg, about 100 mg to about 1,300 mg, about 100 mg to about 1,250 mg, about 100 mg to about 1,200 mg, about 100 mg to about 1,150 mg, about 100 mg to about 1,100 mg, about 100 mg to about 1,050 mg, about 100 mg to about 1,050 mg, about 100 mg to about 1000 mg, about 100 mg to about 950 mg, about 100 mg to about 900 mg, about 100 mg to about 850 mg, about 100 mg to about 800 mg, about 100 mg to about 750 mg, about 100 mg to about 700 mg, about 100 mg to about 650 mg, about 100 mg to about 600 mg, about 100 mg to about 550 mg, about 100 mg to about 500 mg, about 100 mg to about 450 mg, about 100 mg to about 400 mg, about 100 mg to about 350 mg, about 100 mg to about 300 mg, about 100 mg to about 250 mg, about 100 mg to about 200 mg, about 100 mg to about 150 mg, about 200 mg to about 2,000 mg, about 200 mg to about 1,950 mg, about 200 mg to about 1,900 mg, about 200 mg to about 1,850 mg, about 200 mg to about 1,800 mg, about 200 mg to about 1,750 mg, about 200 mg to about 1,700 mg, about 200 mg to about 1,650 mg, about 200 mg to about 1,600 mg, about 200 mg to about 1,550 mg, about 200 mg to about 1,500 mg, about 200 mg to about 1,450 mg, about 200 mg to about 1,400 mg, about 200 mg to about 1,350 mg, about 200 mg to about 1,300 mg, about 200 mg to about 1,250 mg, about 200 mg to about 1,200 mg, about 200 mg to about 1,150 mg, about 200 mg to about 1,100 mg, about 200 mg to about 1,050 mg, about 200 mg to about 1,050 mg, about 200 mg to about 1000 mg, about 200 mg to about 950 mg, about 200 mg to about 900 mg, about 200 mg to about 850 mg, about 200 mg to about 800 mg, about 200 mg to about 750 mg, about 200 mg to about 700 mg, about 200 mg to about 650 mg, about 200 mg to about 600 mg, about 200 mg to about 550 mg, about 200 mg to about 500 mg, about 200 mg to about 450 mg, about 200 mg to about 400 mg, about 200 mg to about 350 mg, about 200 mg to about 300 mg, about 200 mg to about 250 mg, about 300 mg to about 2,000 mg, about 300 mg to about 1,950 mg, about 300 mg to about 1,900 mg, about 300 mg to about 1,850 mg, about 300 mg to about 1,800 mg, about 300 mg to about 1,750 mg, about 300 mg to about 1,700 mg, about 300 mg to about 1,650 mg, about 300 mg to about 1,600 mg, about 300 mg to about 1,550 mg, about 300 mg to about 1,500 mg, about 300 mg to about 1,450 mg, about 300 mg to about 1,400 mg, about 300 mg to about 1,350 mg, about 300 mg to about 1,300 mg, about 300 mg to about 1,250 mg, about 300 mg to about 1,200 mg, about 300 mg to about 1,150 mg, about 300 mg to about 1,100 mg, about 300 mg to about 1,050 mg, about 300 mg to about 1,050 mg, about 300 mg to about 1000 mg, about 300 mg to about 950 mg, about 300 mg to about 900 mg, about 300 mg to about 850 mg, about 300 mg to about 800 mg, about 300 mg to about 750 mg, about 300 mg to about 700 mg, about 300 mg to about 650 mg, about 300 mg to about 600 mg, about 300 mg to about 550 mg, about 300 mg to about 500 mg, about 300 mg to about 450 mg, about 300 mg to about 400 mg, about 300 mg to about 350 mg, about 400 mg to about 2,000 mg, about 400 mg to about 1,950 mg, about 400 mg to about 1,900 mg, about 400 mg to about 1,850 mg, about 400 mg to about 1,800 mg, about 400 mg to about 1,750 mg, about 400 mg to about 1,700 mg, about 400 mg to about 1,650 mg, about 400 mg to about 1,600 mg, about 400 mg to about 1,550 mg, about 400 mg to about 1,500 mg, about 400 mg to about 1,450 mg, about 400 mg to about 1,400 mg, about 400 mg to about 1,350 mg, about 400 mg to about 1,300 mg, about 400 mg to about 1,250 mg, about 400 mg to about 1,200 mg, about 400 mg to about 1,150 mg, about 400 mg to about 1,100 mg, about 400 mg to about 1,050 mg, about 400 mg to about 1,000 mg, about 400 mg to about 950 mg, about 400 mg to about 900 mg, about 400 mg to about 900 mg, about 400 mg to about 850 mg, about 400 mg to about 800 mg, about 400 mg to about 750 mg, about 400 mg to about 700 mg, about 400 mg to about 650 mg, about 400 mg to about 600 mg, about 400 mg to about 550 mg, about 400 mg to about 500 mg, about 400 mg to about 450 mg, about 500 mg to about 2,000 mg, about 500 mg to about 1,950 mg, about 500 mg to about 1,900 mg, about 500 mg to about 1,850 mg, about 500 mg to about 1,800 mg, about 500 mg to about 1,750 mg, about 500 mg to about 1,700 mg, about 500 mg to about 1,650 mg, about 500 mg to about 1,600 mg, about 500 mg to about 1,550 mg, about 500 mg to about 1,500 mg, about 500 mg to about 1,450 mg, about 500 mg to about 1,400 mg, about 500 mg to about 1,350 mg, about 500 mg to about 1,300 mg, about 500 mg to about 1,250 mg, about 500 mg to about 1,200 mg, about 500 mg to about 1,150 mg, about 500 mg to about 1,100 mg, about 500 mg to about 1,050 mg, about 500 mg to about 1,000 mg, about 500 mg to about 950 mg, about 500 mg to about 900 mg, about 500 mg to about 900 mg, about 500 mg to about 850 mg, about 500 mg to about 800 mg, about 500 mg to about 750 mg, about 500 mg to about 700 mg, about 500 mg to about 650 mg, about 500 mg to about 600 mg, about 500 mg to about 550 mg, about 600 mg to about 2,000 mg, about 600 mg to about 1,950 mg, about 600 mg to about 1,900 mg, about 600 mg to about 1,850 mg, about 600 mg to about 1,800 mg, about 600 mg to about 1,750 mg, about 600 mg to about 1,700 mg, about 600 mg to about 1,650 mg, about 600 mg to about 1,600 mg, about 600 mg to about 1,550 mg, about 600 mg to about 1,500 mg, about 600 mg to about 1,450 mg, about 600 mg to about 1,400 mg, about 600 mg to about 1,350 mg, about 600 mg to about 1,300 mg, about 600 mg to about 1,250 mg, about 600 mg to about 1,200 mg, about 600 mg to about 1,150 mg, about 600 mg to about 1,100 mg, about 600 mg to about 1,050 mg, about 600 mg to about 1,000 mg, about 600 mg to about 950 mg, about 600 mg to about 900 mg, about 600 mg to about 900 mg, about 600 mg to about 850 mg, about 600 mg to about 800 mg, about 600 mg to about 750 mg, about 600 mg to about 700 mg, about 600 mg to about 650 mg, about 700 mg to about 2,000 mg, about 700 mg to about 1,950 mg, about 700 mg to about 1,900 mg, about 700 mg to about 1,850 mg, about 700 mg to about 1,800 mg, about 700 mg to about 1,750 mg, about 700 mg to about 1,700 mg, about 700 mg to about 1,650 mg, about 700 mg to about 1,600 mg, about 700 mg to about 1,550 mg, about 700 mg to about 1,500 mg, about 700 mg to about 1,450 mg, about 700 mg to about 1,400 mg, about 700 mg to about 1,350 mg, about 700 mg to about 1,300 mg, about 700 mg to about 1,250 mg, about 700 mg to about 1,200 mg, about 700 mg to about 1,150 mg, about 700 mg to about 1,100 mg, about 700 mg to about 1,050 mg, about 700 mg to about 1,000 mg, about 700 mg to about 950 mg, about 700 mg to about 900 mg, about 700 mg to about 900 mg, about 700 mg to about 850 mg, about 700 mg to about 800 mg, about 700 mg to about 750 mg, about 800 mg to about 2,000 mg, about 800 mg to about 1,950 mg, about 800 mg to about 1,900 mg, about 800 mg to about 1,850 mg, about 800 mg to about 1,800 mg, about 800 mg to about 1,750 mg, about 800 mg to about 1,700 mg, about 800 mg to about 1,650 mg, about 800 mg to about 1,600 mg, about 800 mg to about 1,550 mg, about 800 mg to about 1,500 mg, about 800 mg to about 1,450 mg, about 800 mg to about 1,400 mg, about 800 mg to about 1,350 mg, about 800 mg to about 1,300 mg, about 800 mg to about 1,250 mg, about 800 mg to about 1,200 mg, about 800 mg to about 1,150 mg, about 800 mg to about 1,100 mg, about 800 mg to about 1,050 mg, about 800 mg to about 1,000 mg, about 800 mg to about 950 mg, about 800 mg to about 900 mg, about 800 mg to about 900 mg, about 800 mg to about 850 mg, about 900 mg to about 2,000 mg, about 900 mg to about 1,950 mg, about 900 mg to about 1,900 mg, about 900 mg to about 1,850 mg, about 900 mg to about 1,800 mg, about 900 mg to about 1,750 mg, about 900 mg to about 1,700 mg, about 900 mg to about 1,650 mg, about 900 mg to about 1,600 mg, about 900 mg to about 1,550 mg, about 900 mg to about 1,500 mg, about 900 mg to about 1,450 mg, about 900 mg to about 1,400 mg, about 900 mg to about 1,350 mg, about 900 mg to about 1,300 mg, about 900 mg to about 1,250 mg, about 900 mg to about 1,200 mg, about 900 mg to about 1,150 mg, about 900 mg to about 1,100 mg, about 900 mg to about 1,050 mg, about 900 mg to about 1,000 mg, about 900 mg to about 950 mg, about 1,000 mg to about 2,000 mg, about 1,000 mg to about 1,950 mg, about 1,000 mg to about 1,900 mg, about 1,000 mg to about 1,850 mg, about 1,000 mg to about 1,800 mg, about 1,000 mg to about 1,750 mg, about 1,000 mg to about 1,700 mg, about 1,000 mg to about 1,650 mg, about 1,000 mg to about 1,600 mg, about 1,000 mg to about 1,550 mg, about 1,000 mg to about 1,500 mg, about 1,000 mg to about 1,450 mg, about 1,000 mg to about 1,400 mg, about 1,000 mg to about 1,350 mg, about 1,000 mg to about 1,300 mg, about 1,000 mg to about 1,250 mg, about 1,000 mg to about 1,200 mg, about 1,000 mg to about 1,150 mg, about 1,000 mg to about 1,100 mg, about 1,000 mg to about 1,050 mg, about 1,100 mg to about 2,000 mg, about 1,100 mg to about 1,950 mg, about 1,100 mg to about 1,900 mg, about 1,100 mg to about 1,850 mg, about 1,100 mg to about 1,800 mg, about 1,100 mg to about 1,750 mg, about 1,100 mg to about 1,700 mg, about 1,100 mg to about 1,650 mg, about 1,100 mg to about 1,600 mg, about 1,100 mg to about 1,550 mg, about 1,100 mg to about 1,500 mg, about 1,100 mg to about 1,450 mg, about 1,100 mg to about 1,400 mg, about 1,100 mg to about 1,350 mg, about 1,100 mg to about 1,300 mg, about 1,100 mg to about 1,250 mg, about 1,100 mg to about 1,200 mg, about 1,100 mg to about 1,150 mg, about 1,200 mg to about 2,000 mg, about 1,200 mg to about 1,950 mg, about 1,200 mg to about 1,900 mg, about 1,200 mg to about 1,850 mg, about 1,200 mg to about 1,800 mg, about 1,200 mg to about 1,750 mg, about 1,200 mg to about 1,700 mg, about 1,200 mg to about 1,650 mg, about 1,200 mg to about 1,600 mg, about 1,200 mg to about 1,550 mg, about 1,200 mg to about 1,500 mg, about 1,200 mg to about 1,450 mg, about 1,200 mg to about 1,400 mg, about 1,200 mg to about 1,350 mg, about 1,200 mg to about 1,300 mg, about 1,200 mg to about 1,250 mg, about 1,300 mg to about 2,000 mg, about 1,300 mg to about 1,950 mg, about 1,300 mg to about 1,900 mg, about 1,300 mg to about 1,850 mg, about 1,300 mg to about 1,800 mg, about 1,300 mg to about 1,750 mg, about 1,300 mg to about 1,700 mg, about 1,300 mg to about 1,650 mg, about 1,300 mg to about 1,600 mg, about 1,300 mg to about 1,550 mg, about 1,300 mg to about 1,500 mg, about 1,300 mg to about 1,450 mg, about 1,300 mg to about 1,400 mg, about 1,300 mg to about 1,350 mg, about 1,400 mg to about 2,000 mg, about 1,400 mg to about 1,950 mg, about 1,400 mg to about 1,900 mg, about 1,400 mg to about 1,850 mg, about 1,400 mg to about 1,800 mg, about 1,400 mg to about 1,750 mg, about 1,400 mg to about 1,700 mg, about 1,400 mg to about 1,650 mg, about 1,400 mg to about 1,600 mg, about 1,400 mg to about 1,550 mg, about 1,400 mg to about 1,500 mg, about 1,400 mg to about 1,450 mg, about 1,500 mg to about 2,000 mg, about 1,500 mg to about 1,950 mg, about 1,500 mg to about 1,900 mg, about 1,500 mg to about 1,850 mg, about 1,500 mg to about 1,800 mg, about 1,500 mg to about 1,750 mg, about 1,500 mg to about 1,700 mg, about 1,500 mg to about 1,650 mg, about 1,500 mg to about 1,600 mg, about 1,500 mg to about 1,550 mg, about 1,600 mg to about 2,000 mg, about 1,600 mg to about 1,950 mg, about 1,600 mg to about 1,900 mg, about 1,600 mg to about 1,850 mg, about 1,600 mg to about 1,800 mg, about 1,600 mg to about 1,750 mg, about 1,600 mg to about 1,700 mg, about 1,600 mg to about 1,650 mg, about 1,700 mg to about 2,000 mg, about 1,700 mg to about 1,950 mg, about 1,700 mg to about 1,900 mg, about 1,700 mg to about 1,850 mg, about 1,700 mg to about 1,800 mg, about 1,700 mg to about 1,750 mg, about 1,800 mg to about 2,000 mg, about 1,800 mg to about 1,950 mg, about 1,800 mg to about 1,900 mg, about 1,800 mg to about 1,850 mg, about 1,900 mg to about 2,000 mg, or about 1,900 mg to about 1,950 mg).

In some embodiments, the one or more doses of nirogacestat (e.g., nirogacestat dihydrobromide or nirogacestat hydrobromide) are independently administered to the subject at about 80 mg to about 120 mg of nirogacestat (e.g., nirogacestat dihydrobromide or nirogacestat hydrobromide) (e.g., about 80 mg to about 100 mg, about 80 mg to about 90 mg, about 90 mg to about 120 mg, about 90 mg to about 100 mg, about 100 mg to about 120 mg, about 80 mg, about 90 mg, about 100 mg, about 110 mg, or about 120 mg). In some embodiments, the one or more doses of nirogacestat (e.g., nirogacestat dihydrobromide or nirogacestat hydrobromide) are independently administered to the subject at about 100 mg of nirogacestat (e.g., nirogacestat dihydrobromide or nirogacestat hydrobromide). In some embodiments, two or more doses of about 100 mg nirogacestat (e.g., nirogacestat dihydrobromide or nirogacestat hydrobromide) are independently administered (e.g., orally administered) to the subject twice a day.

### Induction and Maintenance Dosing of BCMA Antibody and Antigen-Binding Fragments Thereof and Nirogacestat

In some embodiments, the methods described herein comprise administering to the subject one or more induction doses of an antibody or an antigen-binding fragment described herein. In some embodiments, the methods described herein further comprise administering to the subject one more maintenance doses of an antibody or an antigen-binding fragment described herein.

In some embodiments, the one or more induction doses are independently administered to the subject at about 100, 200, 400, 800, or 1600 mg of the antibody or antigen-binding fragment, and each dose of nirogacestat is administered to the subject at about 100 mg of nirogacestat. In some embodiments, the one or more induction doses is 800 mg of the antibody or antigen-binding fragment, and each dose of nirogacestat is administered to the subject at about 100 mg of nirogacestat. In further embodiments, the one or more induction doses is 1600 mg of the antibody or antigen-binding fragment, and each dose of nirogacestat is administered to the subject at about 100 mg of nirogacestat.

In some embodiments of any of the methods described herein, the two or more doses of the antibody or the antigen-binding fragment thereof are independently administered to the subject at a frequency of between once a week and about once every four weeks. In some embodiments, the two or more doses of the antibody or the antigen-binding fragment thereof are independently administered to the subject at a frequency of about once a week. In some embodiments, the two or more doses of the antibody or the antigen-binding fragment thereof are independently administered to the subject at a frequency of about once every two weeks, once every three weeks, or once every four weeks.

In some embodiments, each dose of the antibody or the antigen-binding fragment thereof comprises about 100 mg, about 200 mg, about 400 mg, about 800 mg, or about 1600 mg of the antibody or the antigen-binding fragment thereof and is independently administered to the subject about once a week or about once every 2 weeks.

In some embodiments of any of the methods described herein, individual doses of the antibody or antigen-binding fragment thereof are independently administered to the subject on day 1 and day 15 of a 28-day cycle. In some embodiments of any of the methods described herein, individual doses of the antibody or antigen-binding fragment thereof are independently administered to the subject on day 1, day 8, day 15, and day 22 of a 28-day cycle. In some embodiments of any of the methods described herein, the individual doses of the antibody or antigen-binding fragment thereof are independently administered to the subject for multiple 28-day cycles.

In some embodiments, the two or more doses of the antibody or the antigen-binding fragment thereof comprise (1) one or more induction doses that are independently administered to the subject during an induction phase and (2) one or more maintenance doses of the antibody or the antigen-binding fragment thereof that are independently administered to the subject during a maintenance phase after the induction phase. In some embodiments, a single induction dose is administered to the subject. In some embodiments, two or more induction doses are independently administered to the subject. In some embodiments, each of the two or more induction doses are independently administered to the subject about once a week for about 1-10 weeks. In some embodiments, each of the two or more induction doses are independently administered to the subject once a week for 8 weeks. In some embodiments, induction doses are independently administered to the subject 4 times within a 28-day cycle.

In some embodiments, the induction doses are independently administered to the subject 8 times within two 28-day cycles. In some embodiments, the individual induction doses are independently administered to the subject on day 1, day 8, day 15 and day 22 for each of the two 28-day cycles. In some embodiments of any of the methods described herein, each induction dose comprises about 100, about 200, about 400, about 800, or about 1600 mg of the antibody or antigen-binding fragment thereof.

In some embodiments of any of the methods described herein, each induction dose comprises about 100, about 200, about 400, about 800, or about 1600 mg of the antibody or antigen-binding fragment thereof, each maintenance dose comprises about 100, about 200, about 400, about 800, or about 1600 mg of the antibody or antigen-binding fragment thereof, the individual induction doses are independently administered to the subject on each of day 1, day 8, day 15 and day 22 for each of two 28-day cycles for a total of 8 induction doses during the induction phase; and the individual maintenance doses are independently administered to the subject on each of days 1 and day 15 of each of one or more subsequent 28-day cycle(s).

In some embodiments of any of the methods described herein, the dose(s) of the antibody or antigen-binding fragment thereof are administered intravenously to the subject. In some embodiments of any of the methods described herein, a single dose of nirogacestat is administered to the subject. In some embodiments of any of the methods described herein, two or more doses of nirogacestat are independently administered to the subject. In some embodiments of any of the methods described herein, each dose of nirogacestat comprises about 100 mg of nirogacestat. In some embodiments of any of the methods described herein, the two or more doses of nirogacestat are independently administered to the subject at a frequency of about once a day to about four times a day. In some embodiments of any of the methods described herein, the two or more doses of nirogacestat are independently administered to the subject at a frequency of about twice a day. In some embodiments, each dose of nirogacestat comprises about 100 mg of nirogacestat and the two or more doses of nirogacestat are independently administered to the subject at a frequency of about twice a day each day of a 28-day cycle. In some embodiments of any of the methods described herein, the dose(s) of nirogacestat is/are orally administered to the subject.

B. Combination Therapy with Nirogacestat and Dexamethasone

Also provided herein are methods of treating a subject having multiple myeloma (MM) that include administering to the subject one or more doses of an antibody, or antigen binding fragment thereof, that specifically binds to a B cell maturation antigen (BCMA) (e.g., any of the exemplary antibodies or antigen-binding fragments described herein), one or more doses of nirogacestat (e.g., nirogacestat dihydrobromide, nirogacestat hydrobromide), and one or more doses of dexamethasone.

### General Dosing of BCMA Antibody or Antigen-Fragment Thereof, Nirogacestat and Dexamethasone

In some embodiments, the one or more doses are independently administered to the subject at about 100 mg of the antibody or antigen-binding fragment to about 2,000 mg of the antibody or antigen-binding fragment (e.g., about 100 mg to about 2,000 mg, about 100 mg to about 1,950 mg, about 100 mg to about 1,900 mg, about 100 mg to about 1,850 mg, about 100 mg to about 1,800 mg, about 100 mg to about 1,750 mg, about 100 mg to about 1,700 mg, about 100 mg to about 1,650 mg, about 100 mg to about 1,600 mg, about 100 mg to about 1,550 mg, about 100 mg to about 1,500 mg, about 100 mg to about 1,450 mg, about 100 mg to about 1,400 mg, about 100 mg to about 1,350 mg, about 100 mg to about 1,300 mg, about 100 mg to about 1,250 mg, about 100 mg to about 1,200 mg, about 100 mg to about 1,150 mg, about 100 mg to about 1,100 mg, about 100 mg to about 1,050 mg, about 100 mg to about 1,000 mg, about 100 mg to about 950 mg, about 100 mg to about 900 mg, about 100 mg to about 850 mg, about 100 mg to about 800 mg, about 100 mg to about 750 mg, about 100 mg to about 700 mg, about 100 mg to about 650 mg, about 100 mg to about 600 mg, about 100 mg to about 550 mg, about 100 mg to about 500 mg, about 100 mg to about 450 mg, about 100 mg to about 400 mg, about 100 mg to about 350 mg, about 100 mg to about 300 mg, about 100 mg to about 250 mg, about 100 mg to about 200 mg, about 100 mg to about 150 mg, about 200 mg to about 2,000 mg, about 200 mg to about 1,950 mg, about 200 mg to about 1,900 mg, about 200 mg to about 1,850 mg, about 200 mg to about 1,800 mg, about 200 mg to about 1,750 mg, about 200 mg to about 1,700 mg, about 200 mg to about 1,650 mg, about 200 mg to about 1,600 mg, about 200 mg to about 1,550 mg, about 200 mg to about 1,500 mg, about 200 mg to about 1,450 mg, about 200 mg to about 1,400 mg, about 200 mg to about 1,350 mg, about 200 mg to about 1,300 mg, about 200 mg to about 1,250 mg, about 200 mg to about 1,200 mg, about 200 mg to about 1,150 mg, about 200 mg to about 1,100 mg, about 200 mg to about 1,050 mg, about 200 mg to about 1,000 mg, about 200 mg to about 950 mg, about 200 mg to about 900 mg, about 200 mg to about 850 mg, about 200 mg to about 800 mg, about 200 mg to about 750 mg, about 200 mg to about 700 mg, about 200 mg to about 650 mg, about 200 mg to about 600 mg, about 200 mg to about 550 mg, about 200 mg to about 500 mg, about 200 mg to about 450 mg, about 200 mg to about 400 mg, about 200 mg to about 350 mg, about 200 mg to about 300 mg, about 200 mg to about 250 mg, about 300 mg to about 2,000 mg, about 300 mg to about 1,950 mg, about 300 mg to about 1,900 mg, about 300 mg to about 1,850 mg, about 300 mg to about 1,800 mg, about 300 mg to about 1,750 mg, about 300 mg to about 1,700 mg, about 300 mg to about 1,650 mg, about 300 mg to about 1,600 mg, about 300 mg to about 1,550 mg, about 300 mg to about 1,500 mg, about 300 mg to about 1,450 mg, about 300 mg to about 1,400 mg, about 300 mg to about 1,350 mg, about 300 mg to about 1,300 mg, about 300 mg to about 1,250 mg, about 300 mg to about 1,200 mg, about 300 mg to about 1,150 mg, about 300 mg to about 1,100 mg, about 300 mg to about 1,050 mg, about 300 mg to about 1,000 mg, about 300 mg to about 950 mg, about 300 mg to about 900 mg, about 300 mg to about 850 mg, about 300 mg to about 800 mg, about 300 mg to about 750 mg, about 300 mg to about 700 mg, about 300 mg to about 650 mg, about 300 mg to about 600 mg, about 300 mg to about 550 mg, about 300 mg to about 500 mg, about 300 mg to about 450 mg, about 300 mg to about 400 mg, about 300 mg to about 350 mg, about 400 mg to about 2,000 mg, about 400 mg to about 1,950 mg, about 400 mg to about 1,900 mg, about 400 mg to about 1,850 mg, about 400 mg to about 1,800 mg, about 400 mg to about 1,750 mg, about 400 mg to about 1,700 mg, about 400 mg to about 1,650 mg, about 400 mg to about 1,600 mg, about 400 mg to about 1,550 mg, about 400 mg to about 1,500 mg, about 400 mg to about 1,450 mg, about 400 mg to about 1,400 mg, about 400 mg to about 1,350 mg, about 400 mg to about 1,300 mg, about 400 mg to about 1,250 mg, about 400 mg to about 1,200 mg, about 400 mg to about 1,150 mg, about 400 mg to about 1,100 mg, about 400 mg to about 1,050 mg, about 400 mg to about 1,000 mg, about 400 mg to about 950 mg, about 400 mg to about 900 mg, about 400 mg to about 900 mg, about 400 mg to about 850 mg, about 400 mg to about 800 mg, about 400 mg to about 750 mg, about 400 mg to about 700 mg, about 400 mg to about 650 mg, about 400 mg to about 600 mg, about 400 mg to about 550 mg, about 400 mg to about 500 mg, about 400 mg to about 450 mg, about 500 mg to about 2,000 mg, about 500 mg to about 1,950 mg, about 500 mg to about 1,900 mg, about 500 mg to about 1,850 mg, about 500 mg to about 1,800 mg, about 500 mg to about 1,750 mg, about 500 mg to about 1,700 mg, about 500 mg to about 1,650 mg, about 500 mg to about 1,600 mg, about 500 mg to about 1,550 mg, about 500 mg to about 1,500 mg, about 500 mg to about 1,450 mg, about 500 mg to about 1,400 mg, about 500 mg to about 1,350 mg, about 500 mg to about 1,300 mg, about 500 mg to about 1,250 mg, about 500 mg to about 1,200 mg, about 500 mg to about 1,150 mg, about 500 mg to about 1,100 mg, about 500 mg to about 1,050 mg, about 500 mg to about 1,000 mg, about 500 mg to about 950 mg, about 500 mg to about 900 mg, about 500 mg to about 900 mg, about 500 mg to about 850 mg, about 500 mg to about 800 mg, about 500 mg to about 750 mg, about 500 mg to about 700 mg, about 500 mg to about 650 mg, about 500 mg to about 600 mg, about 500 mg to about 550 mg, about 600 mg to about 2,000 mg, about 600 mg to about 1,950 mg, about 600 mg to about 1,900 mg, about 600 mg to about 1,850 mg, about 600 mg to about 1,800 mg, about 600 mg to about 1,750 mg, about 600 mg to about 1,700 mg, about 600 mg to about 1,650 mg, about 600 mg to about 1,600 mg, about 600 mg to about 1,550 mg, about 600 mg to about 1,500 mg, about 600 mg to about 1,450 mg, about 600 mg to about 1,400 mg, about 600 mg to about 1,350 mg, about 600 mg to about 1,300 mg, about 600 mg to about 1,250 mg, about 600 mg to about 1,200 mg, about 600 mg to about 1,150 mg, about 600 mg to about 1,100 mg, about 600 mg to about 1,050 mg, about 600 mg to about 1,000 mg, about 600 mg to about 950 mg, about 600 mg to about 900 mg, about 600 mg to about 900 mg, about 600 mg to about 850 mg, about 600 mg to about 800 mg, about 600 mg to about 750 mg, about 600 mg to about 700 mg, about 600 mg to about 650 mg, about 700 mg to about 2,000 mg, about 700 mg to about 1,950 mg, about 700 mg to about 1,900 mg, about 700 mg to about 1,850 mg, about 700 mg to about 1,800 mg, about 700 mg to about 1,750 mg, about 700 mg to about 1,700 mg, about 700 mg to about 1,650 mg, about 700 mg to about 1,600 mg, about 700 mg to about 1,550 mg, about 700 mg to about 1,500 mg, about 700 mg to about 1,450 mg, about 700 mg to about 1,400 mg, about 700 mg to about 1,350 mg, about 700 mg to about 1,300 mg, about 700 mg to about 1,250 mg, about 700 mg to about 1,200 mg, about 700 mg to about 1,150 mg, about 700 mg to about 1,100 mg, about 700 mg to about 1,050 mg, about 700 mg to about 1,000 mg, about 700 mg to about 950 mg, about 700 mg to about 900 mg, about 700 mg to about 900 mg, about 700 mg to about 850 mg, about 700 mg to about 800 mg, about 700 mg to about 750 mg, about 800 mg to about 2,000 mg, about 800 mg to about 1,950 mg, about 800 mg to about 1,900 mg, about 800 mg to about 1,850 mg, about 800 mg to about 1,800 mg, about 800 mg to about 1,750 mg, about 800 mg to about 1,700 mg, about 800 mg to about 1,650 mg, about 800 mg to about 1,600 mg, about 800 mg to about 1,550 mg, about 800 mg to about 1,500 mg, about 800 mg to about 1,450 mg, about 800 mg to about 1,400 mg, about 800 mg to about 1,350 mg, about 800 mg to about 1,300 mg, about 800 mg to about 1,250 mg, about 800 mg to about 1,200 mg, about 800 mg to about 1,150 mg, about 800 mg to about 1,100 mg, about 800 mg to about 1,050 mg, about 800 mg to about 1,000 mg, about 800 mg to about 950 mg, about 800 mg to about 900 mg, about 800 mg to about 900 mg, about 800 mg to about 850 mg, about 900 mg to about 2,000 mg, about 900 mg to about 1,950 mg, about 900 mg to about 1,900 mg, about 900 mg to about 1,850 mg, about 900 mg to about 1,800 mg, about 900 mg to about 1,750 mg, about 900 mg to about 1,700 mg, about 900 mg to about 1,650 mg, about 900 mg to about 1,600 mg, about 900 mg to about 1,550 mg, about 900 mg to about 1,500 mg, about 900 mg to about 1,450 mg, about 900 mg to about 1,400 mg, about 900 mg to about 1,350 mg, about 900 mg to about 1,300 mg, about 900 mg to about 1,250 mg, about 900 mg to about 1,200 mg, about 900 mg to about 1,150 mg, about 900 mg to about 1,100 mg, about 900 mg to about 1,050 mg, about 900 mg to about 1,000 mg, about 900 mg to about 950 mg, about 1,000 mg to about 2,000 mg, about 1,000 mg to about 1,950 mg, about 1,000 mg to about 1,900 mg, about 1,000 mg to about 1,850 mg, about 1,000 mg to about 1,800 mg, about 1,000 mg to about 1,750 mg, about 1,000 mg to about 1,700 mg, about 1,000 mg to about 1,650 mg, about 1,000 mg to about 1,600 mg, about 1,000 mg to about 1,550 mg, about 1,000 mg to about 1,500 mg, about 1,000 mg to about 1,450 mg, about 1,000 mg to about 1,400 mg, about 1,000 mg to about 1,350 mg, about 1,000 mg to about 1,300 mg, about 1,000 mg to about 1,250 mg, about 1,000 mg to about 1,200 mg, about 1,000 mg to about 1,150 mg, about 1,000 mg to about 1,100 mg, about 1,000 mg to about 1,050 mg, about 1,100 mg to about 2,000 mg, about 1,100 mg to about 1,950 mg, about 1,100 mg to about 1,900 mg, about 1,100 mg to about 1,850 mg, about 1,100 mg to about 1,800 mg, about 1,100 mg to about 1,750 mg, about 1,100 mg to about 1,700 mg, about 1,100 mg to about 1,650 mg, about 1,100 mg to about 1,600 mg, about 1,100 mg to about 1,550 mg, about 1,100 mg to about 1,500 mg, about 1,100 mg to about 1,450 mg, about 1,100 mg to about 1,400 mg, about 1,100 mg to about 1,350 mg, about 1,100 mg to about 1,300 mg, about 1,100 mg to about 1,250 mg, about 1,100 mg to about 1,200 mg, about 1,100 mg to about 1,150 mg, about 1,200 mg to about 2,000 mg, about 1,200 mg to about 1,950 mg, about 1,200 mg to about 1,900 mg, about 1,200 mg to about 1,850 mg, about 1,200 mg to about 1,800 mg, about 1,200 mg to about 1,750 mg, about 1,200 mg to about 1,700 mg, about 1,200 mg to about 1,650 mg, about 1,200 mg to about 1,600 mg, about 1,200 mg to about 1,550 mg, about 1,200 mg to about 1,500 mg, about 1,200 mg to about 1,450 mg, about 1,200 mg to about 1,400 mg, about 1,200 mg to about 1,350 mg, about 1,200 mg to about 1,300 mg, about 1,200 mg to about 1,250 mg, about 1,300 mg to about 2,000 mg, about 1,300 mg to about 1,950 mg, about 1,300 mg to about 1,900 mg, about 1,300 mg to about 1,850 mg, about 1,300 mg to about 1,800 mg, about 1,300 mg to about 1,750 mg, about 1,300 mg to about 1,700 mg, about 1,300 mg to about 1,650 mg, about 1,300 mg to about 1,600 mg, about 1,300 mg to about 1,550 mg, about 1,300 mg to about 1,500 mg, about 1,300 mg to about 1,450 mg, about 1,300 mg to about 1,400 mg, about 1,300 mg to about 1,350 mg, about 1,400 mg to about 2,000 mg, about 1,400 mg to about 1,950 mg, about 1,400 mg to about 1,900 mg, about 1,400 mg to about 1,850 mg, about 1,400 mg to about 1,800 mg, about 1,400 mg to about 1,750 mg, about 1,400 mg to about 1,700 mg, about 1,400 mg to about 1,650 mg, about 1,400 mg to about 1,600 mg, about 1,400 mg to about 1,550 mg, about 1,400 mg to about 1,500 mg, about 1,400 mg to about 1,450 mg, about 1,500 mg to about 2,000 mg, about 1,500 mg to about 1,950 mg, about 1,500 mg to about 1,900 mg, about 1,500 mg to about 1,850 mg, about 1,500 mg to about 1,800 mg, about 1,500 mg to about 1,750 mg, about 1,500 mg to about 1,700 mg, about 1,500 mg to about 1,650 mg, about 1,500 mg to about 1,600 mg, about 1,500 mg to about 1,550 mg, about 1,600 mg to about 2,000 mg, about 1,600 mg to about 1,950 mg, about 1,600 mg to about 1,900 mg, about 1,600 mg to about 1,850 mg, about 1,600 mg to about 1,800 mg, about 1,600 mg to about 1,750 mg, about 1,600 mg to about 1,700 mg, about 1,600 mg to about 1,650 mg, about 1,700 mg to about 2,000 mg, about 1,700 mg to about 1,950 mg, about 1,700 mg to about 1,900 mg, about 1,700 mg to about 1,850 mg, about 1,700 mg to about 1,800 mg, about 1,700 mg to about 1,750 mg, about 1,800 mg to about 2,000 mg, about 1,800 mg to about 1,950 mg, about 1,800 mg to about 1,900 mg, about 1,800 mg to about 1,850 mg, about 1,900 mg to about 2,000 mg, or about 1,900 mg to about 1,950 mg).

In some embodiments, the one or more doses of nirogacestat (e.g., nirogacestat dihydrobromide or nirogacestat hydrobromide) are independently administered to the subject at about 80 mg to about 120 mg of nirogacestat (e.g., about 80 mg to about 100 mg, about 80 mg to about 90 mg, about 90 mg to about 120 mg, about 90 mg to about 100 mg, about 100 mg to about 120 mg, about 80 mg, about 90 mg, about 100 mg, about 110 mg, or about 120 mg). In some embodiments, the one or more doses of nirogacestat (e.g., nirogacestat dihydrobromide or nirogacestat hydrobromide) are independently administered to the subject at about 100 mg of nirogacestat (e.g., nirogacestat dihydrobromide or nirogacestat hydrobromide). In some embodiments, two or more doses of about 100 mg nirogacestat (e.g., nirogacestat dihydrobromide or nirogacestat hydrobromide) are independently administered (e.g., orally administered) to the subject twice a day.

In some embodiments, the one or more doses of dexamethasone are independently administered to the subject at about 5 mg to about 200 mg (e.g., about 5 mg to about 150 mg, about 5 mg to about 100 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg. about 10 mg to about 200 mg, about 10 mg to about 150 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 200 mg, about 20 mg to about 150 mg, about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 200 mg, about 30 mg to about 150 mg, about 30 mg to about 100 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 200 mg, about 40 mg to about 150 mg, about 40 mg to about 100 mg, about 40 mg to about 80 mg, about 40 mg to about 60 mg, about 40 mg to about 50 mg, about 50 mg to about 200 mg, about 50 mg to about 150 mg, about 50 mg to about 100 mg, about 50 mg to about 90 mg, about 50 mg to about 80 mg, about 50 mg to about 70 mg, about 50 mg to about 60 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 115 mg, about 120 mg, about 140 mg, about 150 mg, about 170 mg, about 180 mg, or about 200 mg).

### Induction and Maintenance Dosing of BCMA Antibody and Antigen-Binding Fragments Thereof, Nirogacestat and Dexamethasone

In some embodiments, each of two or more doses of the antibody or antigen-binding fragment thereof are independently administered to the subject at a frequency of about once every 1-4 weeks; each dose of nirogacestat is independently administered to the subject at a frequency of once a day to about four times a day; and each dose of dexamethasone is independently administered to the subject at a frequency of about once every 1-4 weeks.

In some embodiments, each dose of the antibody or antigen-binding fragment thereof is independently administered to the subject about once every two weeks; each dose of nirogacestat is independently administered to the subject twice a day; and each dose of dexamethasone is independently administered to the subject about once a week.

In some embodiments, each dose of the antibody or antigen-binding fragment thereof is independently administered to the subject on each of day 1 and day 15 of one or more 28-day cycle(s); each dose of nirogacestat is independently administered to the subject on each of day 1 to day 28 of the one or more 28-day cycle(s); and each dose of dexamethasone is independently administered to the subject on each of day 1, day 8, day 15 and day 22 of the one or more 28-day cycle(s).

In some embodiments, each dose of the antibody or antigen-binding fragment comprises about 400-1600 mg (or any of the subranges of this range described herein) of the antibody or antigen-binding fragment thereof, each dose of nirogacestat comprises about 100 mg of nirogacestat, and each dose of dexamethasone comprises about 40 mg of dexamethasone.

In some embodiments, each dose of the antibody or antigen-binding fragment comprises about 400 mg of the antibody or antigen-binding fragment thereof, each dose of nirogacestat comprises about 100 mg of nirogacestat, and each of dose of dexamethasone comprises about 40 mg of dexamethasone.

In some embodiments, each dose of the antibody or antigen-binding fragment comprises about 800 mg of the antibody or antigen-binding fragment thereof, each dose of nirogacestat comprises about 100 mg of nirogacestat, and each of dose of dexamethasone comprises about 40 mg of dexamethasone.

In some embodiments, each dose of the antibody or antigen-binding fragment comprises about 1600 mg of the antibody or antigen-binding fragment thereof, each dose of nirogacestat comprises about 100 mg of nirogacestat, and each of dose of dexamethasone comprises about 40 mg of dexamethasone.

In some embodiments, two or more doses of the antibody or antigen-binding fragment thereof are independently administered to the subject at a frequency of about once a week during an induction phase, and two or more doses of the antibody or antigen-binding fragment thereof are independently administered to the subject at a frequency of about once every two weeks during a subsequent maintenance phase; two or more doses of nirogacestat are independently administered to the subject at a frequency of about twice a day during one or both of the induction phase and the maintenance phase; and two or more doses of dexamethasone are independently administered to the subject at a frequency of about once a week during one or both of the induction phase and the maintenance phase. In some embodiments, the induction phase is about 8 weeks.

In some embodiments, two or more doses of the antibody or antigen-binding fragment thereof are independently administered to the subject on each of day 1, day 8, day 15, and day 22 of each of two 28-day cycles of the induction phase and then on each of day 1 and day 15 of subsequent 28-day cycle(s) of the maintenance phase; two or more doses of nirogacestat are independently administered to the subject on each of day 1 to day 28 of each of the two 28-day cycles of the induction phase and each of the subsequent 28-day cycle(s) of the maintenance phase; and two or more doses of dexamethasone are independently administered to the subject on each of day 1, day 8, day 15, and day 22 of each of the two 28-day cycles of the induction phase and each of the subsequent 28-day cycle(s) of the maintenance phase.

In some embodiments, the two or more doses of the antibody or antigen-binding fragment independently administered to the subject on each of day 1, day 8, day 15, and day 22 of each of the two 28-day cycles of the induction phase comprises about 100 mg, about 200 mg, about 400 mg, about 800 mg, or about 1600 mg of the antibody or antigen-binding fragment thereof, the two or more doses of the antigen or antigen-binding fragment independently administered to the subject on each of day 1 and day 15 of each of the subsequent 28-day cycle(s) of the maintenance phase comprises about 100, about 200, about 400, about 800, or about 1600 mg; the two or more doses of nirogacestat independently administered to the subject on each of day 1 to day 28 of each of the two 28-day cycles of the induction phase and each of the subsequent 28-day cycle(s) of the maintenance phase comprises about 80 mg to about 120 mg of nirogacestat; and the two or more doses of dexamethasone independently administered to the subject on each of day 1, day 8, day 15, and day 22 of each of the two 28-day cycles of the induction phase and each of the subsequent 28-day cycle(s) of the maintenance phase comprises about 20 mg to about 60 mg of dexamethasone.

In some embodiments, the two or more doses of the antibody or antigen-binding fragment thereof independently administered to the subject on each of day 1, day 8, day 15, and day 22 of each of the two 28-day cycles of the induction phase comprises about 800 mg of the antibody or antigen-binding fragment thereof.

In some embodiments, the two or more doses of the antibody or antigen-binding fragment thereof independently administered to the subject on each of day 1, day 8, day 15, and day 22 of each of the two 28-day cycles of the induction phase comprises about 1,600 mg of the antibody or antigen-binding fragment thereof.

In some embodiments of any of the methods described herein, the two or more doses of nirogacestat independently administered to the subject on each of day 1 to day 28 of each of the two 28-day cycles of the induction phase and each of the subsequent 28-day cycle(s) of the maintenance phase comprises about 100 mg of nirogacestat.

In some embodiments of any of the methods described herein, the two or more doses of dexamethasone independently administered to the subject on each of day 1, day 8, day 15, and day 22 of each of the two 28-day cycles of the induction phase and each of the subsequent 28-day cycle(s) of the maintenance phase comprises about 20 mg dexamethasone.

In some embodiments of any of the methods described herein, the two or more doses of dexamethasone independently administered to the subject on each of day 1, day 8, day 15, and day 22 of each of the two 28-day cycles of the induction phase and each of the subsequent 28-day cycle(s) of the maintenance phase comprises about 40 mg dexamethasone.

In some embodiments, the two or more doses of the antibody or antigen-binding fragment independently administered to the subject on each of day 1, day 8, day 15, and day 22 of each of the two 28-day cycles of the induction phase comprises about 1600 mg of the antibody or antigen-binding fragment; the two or more doses of the antibody or antigen-binding fragment independently administered to the subject on each of day 1 and day 15 of each of the subsequent 28-day cycle(s) of the maintenance phase comprises about 1600 mg; the two or more doses of nirogacestat independently administered to the subject on each of day 1 to day 28 of each of the 28-day cycles of the induction phase and each of the subsequent 28-day cycle(s) of the maintenance phase comprises about 100 mg of nirogacestat; and the two or more doses of dexamethasone independently administered to the subject on each of day 1, day 8, day 15, and day 22 of each of the two 28-day cycles of the induction phase and each of the subsequent 28-day cycle(s) of the maintenance phase comprises about 40 mg of dexamethasone.

In some embodiments of any of the methods described herein, the two or more doses of the antibody or antigen-binding fragment independently administered to the subject on each of day 1, day 8, day 15, and day 22 of each of the two 28-day cycles of the induction phase comprises about 800 mg of the antibody or antigen-binding fragment; the two or more doses of the antibody or antigen-binding fragment independently administered to the subject on each of day 1 and day 15 of each of the subsequent 28-day cycle(s) of the maintenance phase comprises about 800 mg; the two or more doses of nirogacestat independently administered to the subject on each of day 1 to day 28 of each of the 28-day cycles of the induction phase and each of the subsequent 28-day cycle(s) of the maintenance phase comprises about 100 mg of nirogacestat; and the two or more doses of dexamethasone independently administered to the subject on each of day 1, day 8, day 15, and day 22 of each of the two 28-day cycles of the induction phase and each of the subsequent 28-day cycle(s) of the maintenance phase comprises about 40 mg of dexamethasone.

In some embodiments, the dexamethasone is administered to the subject about 10 minutes to about 5 hours (e.g., about 5 minutes to about 4.5 hours, about 5 minutes to about 4 hours, about 5 minutes to about 3.5 hours, about 5 minutes to about 3 hours, about 5 minutes to about 2.5 hours, about 5 minutes to about 2 hours, about 5 minutes to about 1.5 hours, about 5 minutes to about 1 hours, about 5 minutes to about 45 minutes, about 5 minutes to about 40 minutes, about 5 minutes to about 35 minutes, about 5 minutes to about 30 minutes, about 5 minutes to about 25 minutes, about 5 minutes to about 20 minutes, about 5 minutes to about 15 minutes, about 5 minutes to about 10 minutes, about 30 minutes to about 5 hours, about 30 minutes to about 4.5 hours, about 30 minutes to about 4 hours, about 30 minutes to about 3.5 hours, about 30 minutes to about 3 hours, about 30 minutes to about 2.5 hours, about 30 minutes to about 2 hours, about 30 minutes to about 1.5 hours, about 30 minutes to about 1 hours, about 30 minutes to about 45 minutes, about 1 hour to about 5 hours, about 1 hour to about 4.5 hours, about 1 hour to about 4 hours, about 1 hour to about 3.5 hours, about 1 hour to about 3 hours, about 1 hour to about 2.5 hours, about 1 hour to about 2 hours, about 1 hour to about 1.5 hours) prior to the administration of each dose of the pharmaceutical composition described herein (e.g., comprising any of the antibodies or antigen-binding fragments described herein).

In some embodiments, the dexamethasone is administered to the subject about 10 minutes to about 5 hours (e.g., about 5 minutes to about 4.5 hours, about 5 minutes to about 4 hours, about 5 minutes to about 3.5 hours, about 5 minutes to about 3 hours, about 5 minutes to about 2.5 hours, about 5 minutes to about 2 hours, about 5 minutes to about 1.5 hours, about 5 minutes to about 1 hours, about 5 minutes to about 45 minutes, about 5 minutes to about 40 minutes, about 5 minutes to about 35 minutes, about 5 minutes to about 30 minutes, about 5 minutes to about 25 minutes, about 5 minutes to about 20 minutes, about 5 minutes to about 15 minutes, about 5 minutes to about 10 minutes, about 30 minutes to about 5 hours, about 30 minutes to about 4.5 hours, about 30 minutes to about 4 hours, about 30 minutes to about 3.5 hours, about 30 minutes to about 3 hours, about 30 minutes to about 2.5 hours, about 30 minutes to about 2 hours, about 30 minutes to about 1.5 hours, about 30 minutes to about 1 hours, about 30 minutes to about 45 minutes, about 1 hour to about 5 hours, about 1 hour to about 4.5 hours, about 1 hour to about 4 hours, about 1 hour to about 3.5 hours, about 1 hour to about 3 hours, about 1 hour to about 2.5 hours, about 1 hour to about 2 hours, about 1 hour to about 1.5 hours) after the administration of each dose of the pharmaceutical composition described herein (e.g., comprising any of the antibodies or antigen-binding fragments described herein).

In some embodiments, a dose of about 40 mg of dexamethasone is administered to the subject about 1 to about 3 hours prior to each dose of the pharmaceutical composition described herein (e.g., comprising any of the antibodies or antigen-binding fragments described herein).

### C. Treatment Period

In some embodiments, the treatment period can be about 1 week to about 5 years (e.g., about 1 week to about 4.5 years, about 1 week to about 4 years, about 1 week to about 3.5 years, about 1 week to about 3 years, about 1 week to about 2.5 years, about 1 week to about 2 years, about 1 week to about 1.5 years, about 1 week to about 1 year, about 1 week to about 10 months, about 1 week to about 8 months, about 1 week to about 6 months, about 1 week to about 4 months, about 1 week to about 2 months, about 1 week to about 1 month, about 1 week to about 2 weeks, about 2 weeks to about 5 years, about 2 weeks to about 4.5 years, about 2 weeks to about 4 years, about 2 weeks to about 3.5 years, about 2 weeks to about 3 years, about 2 weeks to about 2.5 years, about 2 weeks to about 2 years, about 2 weeks to about 1.5 years, about 2 weeks to about 1 year, about 2 weeks to about 10 months, about 2 weeks to about 8 months, about 2 weeks to about 6 months, about 2 weeks to about 4 months, about 2 weeks to about 2 months, about 2 weeks to about 1 month, about 1 month to about 5 years, about 1 month to about 4.5 years, about 1 month to about 4 years, about 1 month to about 3.5 years, about 1 month to about 3 years, about 1 month to about 2.5 years, about 1 month to about 2 years, about 1 month to about 1.5 years, about 1 month to about 1 year, about 1 month to about 10 months, about 1 month to about 8 months, about 1 month to about 6 months, about 1 month to about 4 months, about 1 month to about 2 months, about 2 months to about 5 years, about 2 months to about 4.5 years, about 2 months to about 4 years, about 2 months to about 3.5 years, about 2 months to about 3 years, about 2 months to about 2.5 years, about 2 months to about 2 years, about 2 months to about 1.5 years, about 2 months to about 1 year, about 2 months to about 10 months, about 2 months to about 8 months, about 2 months to about 6 months, about 2 months to about 4 months, about 4 months to about 5 years, about 4 months to about 4.5 years, about 4 months to about 4 years, about 4 months to about 3.5 years, about 4 months to about 3 years, about 4 months to about 2.5 years, about 4 months to about 2 years, about 4 months to about 1.5 years, about 4 months to about 1 year, about 4 months to about 10 months, about 4 months to about 8 months, about 4 months to about 6 months, about 6 months to about 5 years, about 6 months to about 4.5 years, about 6 months to about 4 years, about 6 months to about 3.5 years, about 6 months to about 3 years, about 6 months to about 2.5 years, about 6 months to about 2 years, about 6 months to about 1.5 years, about 6 months to about 1 year, about 6 months to about 10 months, about 6 months to about 8 months, about 8 months to about 5 years, about 8 months to about 4.5 years, about 8 months to about 4 years, about 8 months to about 3.5 years, about 8 months to about 3 years, about 8 months to about 2.5 years, about 8 months to about 2 years, about 8 months to about 1.5 years, about 8 months to about 1 year, about 8 months to about 10 months, about 10 months to about 5 years, about 10 months to about 4.5 years, about 10 months to about 4 years, about 10 months to about 3.5 years, about 10 months to about 3 years, about 10 months to about 2.5 years, about 10 months to about 2 years, about 10 months to about 1.5 years, about 10 months to about 1 year, about 1 year to about 5 years, about 1 year to about 4.5 years, about 1 year to about 4 years, about 1 year to about 3.5 years, about 1 year to about 3 years, about 1 year to about 2.5 years, about 1 year to about 2 years, about 1 year to about 1.5 years, about 1.5 years to about 5 years, about 1.5 years to about 4.5 years, about 1.5 years to about 4 years, about 1.5 years to about 3.5 years, about 1.5 years to about 3 years, about 1.5 years to about 2.5 years, about 1.5 years to about 2 years, about 2 years to about 5 years, about 2 years to about 4.5 years, about 2 years to about 4 years, about 2 years to about 3.5 years, about 2 years to about 3 years, about 2 years to about 2.5 years, about 2.5 years to about 5 years, about 2.5 years to about 4.5 years, about 2.5 years to about 4 years, about 2/5 years to about 3.5 years, about 2.5 years to about 3 years, about 3 years to about 5 years, about 3 years to about 4.5 years, about 3 years to about 4 years, about 3 years to about 3.5 years, about 3.5 years to about 5 years, about 3.5 years to about 4.5 years, about 3.5 years to about 4 years, about 4 years to about 5 years, about 4 years to about 4.5 years, or about 4.5 years to about 5 years).

An effective treatment of multiple myeloma in a subject means one or more of a reduction in the severity of the disease, a decrease in the rate of development, and/or a reduction in one or more of the number, frequency, severity, and/or duration of one or more symptoms of multiple myeloma in a subject. In some instances, therapeutic efficacy can be observed in a subject relative to historical controls or past experience in the same subject. In other instances, therapeutic efficacy can be demonstrated in a preclinical or clinical trial in a population of treated subjects relative to a control population of untreated or placebo-treated subjects.

In some embodiments, a pharmaceutical composition comprising any of the antibodies or antigen-binding fragments described herein is administered at a frequency of once every two weeks. In some embodiments, a pharmaceutical composition comprising any of the antibodies or antigen-binding fragments described herein is administered at a 1600 mg fixed dose once a week. In some embodiments, a pharmaceutical composition comprising any of the antibodies or antigen-binding fragments described herein is administered at a 1600 mg fixed dose once every two weeks. In some embodiments, a pharmaceutical composition comprising any of the antibodies or antigen-binding fragments described herein is administered at an 800 mg fixed dose once a week. In some embodiments, a pharmaceutical composition comprising any of the antibodies or antigen-binding fragments described herein is administered at an 800 mg fixed dose once every two weeks.

In some embodiments, provided herein are methods of treating a subject having multiple myeloma, the method including administering to the subject (i) one or more doses of a pharmaceutical composition comprising an antibody, or antigen-binding fragment thereof, that specifically binds to a B cell maturation antigen (BCMA), and (ii) one or more doses of a pharmaceutical composition comprising nirogacestat, and optionally (iii) one or more doses of a pharmaceutical composition comprising dexamethasone. In some embodiments, the multiple myeloma is relapsed or refractory multiple myeloma (RRMM). In some embodiments, the antibody, or antigen-binding fragment thereof comprises: a heavy chain variable region comprising a CDR1 comprising SEQ ID NO: 1, a CDR2 comprising SEQ ID NO: 2, and a CDR3 comprising SEQ ID NO: 3, and a light chain variable domain comprising a CDR1 comprising SEQ ID NO: 5, a CDR2 comprising SEQ ID NO: 6, and a CDR3 comprising SEQ ID NO: 7. In some embodiments, the antibody is an IgG1 antibody.

In some embodiments, one or more doses of 1600 mg of the antibody, or antigen-binding fragment thereof, is independently administered to the subject at a frequency of every two weeks. In some embodiments, one or more doses of 800 mg of the antibody, or antigen-binding fragment thereof, is independently administered to the subject at a frequency of every week. In some embodiments, about 1-2 induction doses of 1600 mg of the antibody, or antigen-binding fragment thereof, is independently administered to the subject at a frequency of every week, followed by one or more maintenance doses of 1600 mg of the antibody, or antigen-binding fragment thereof, independently administered to the subject at a frequency of every two weeks. In some embodiments, about 1-2 induction doses of 800 mg of the antibody, or antigen-binding fragment thereof, is independently administered to the subject at a frequency of every week, followed by one or more maintenance doses of 1600 mg of the antibody, or antigen-binding fragment thereof, independently administered to the subject at a frequency of every two weeks.

In some embodiments, the subject was previously administered one or more therapeutic agents or treatments for multiple myeloma. The one or more previously administered therapeutic agents or treatments for multiple myeloma include, but are not limited to, a proteasome inhibitor (PI), an immunomodulatory drug (IMiD), and an anti-CD38 antibody. In some embodiments, the subject has previously been administered a BCMA-directed myeloma therapy other than at least one of a proteasome inhibitor, an immunomodulatory agent, and an anti-CD38 antibody, or cannot tolerate any of the foregoing.

Specifically, proteasome inhibitors are agents whose mechanism of action is to inhibit a proteasome. Exemplary proteasome inhibitors include, but are not limited to are bortezomib, carfilzomib, and ixazomib. Immunomodulatory drugs (IMiDs) are thalidomide analogues, which possess pleiotropic anti-myeloma properties including immune-modulation, anti-angiogenic, anti-inflammatory and anti-proliferative effects. Immunomodulatory imide drugs (IMiDs) are immunomodulatory agents containing and “imide” group. Exemplary IMiDs include, but are not limited to, lenalidomide, pomalidomide, thalidomide, and Iberdomide (CC-220, Celgene). Exemplary anti-CD38 antibodies include, but are not limited to, daratumumab and isatuximab.

In some embodiments, the previously administered one or more therapeutic agents or treatments were not effective in treating the multiple myeloma. In some embodiments, the subject has one or more measurable diseases including a serum monoclonal paraprotein (M-protein) level of ≥0.5 g/dL, a urine M-protein level of ≥200 mg/24 hours, a serum immunoglobulin free light chain ≥10 mg/dL, and/or an abnormal serum immunoglobulin kappa to lambda free light chain ratio.

### D. Routes of Administration

Administration of a pharmaceutical composition (e.g., any of the exemplary pharmaceutical compositions described herein comprising any of the antibodies or antigen-binding fragments described herein, or any of the exemplary pharmaceutical compositions described herein comprising dexamethasone) can be parenteral. In some embodiments, administration of a pharmaceutical composition (e.g., any of the exemplary pharmaceutical compositions described herein comprising any of the antibodies or antigen-binding fragments described herein, or any of the exemplary pharmaceutical compositions described herein comprising dexamethasone) can be intravenous, subcutaneous, intra-arterial, intracranial, intrathecal, intraperitoneal, or intramuscular. Administration can also be localized directly into a tumor. Administration into the systemic circulation by intravenous or subcutaneous administration. In some embodiments, the administration of a pharmaceutical composition (e.g., any of the exemplary pharmaceutical compositions described herein comprising any of the antibodies or antigen-binding fragments described herein or any of the exemplary pharmaceutical compositions described herein comprising dexamethasone) is systemic. In some embodiments, the systemic administration of a pharmaceutical composition (e.g., any of the exemplary pharmaceutical compositions described herein comprising any of the antibodies or antigen-binding fragments described herein, or any of the exemplary pharmaceutical compositions described herein comprising dexamethasone) is intravenous administration.

Intravenous administration can be performed, for example, by step-wise infusion or a single bolus injection. In some embodiments, the step-wise infusion is performed using an infusion rate of about 20 mg/hour to about 500 mg/hour (e.g., about 20 mg/hour to about 450 mg/hour, about 20 mg/hour to about 400 mg/hour, about 20 mg/hour to about 350 mg/hour, about 20 mg/hour to about 300 mg/hour, about 20 mg/hour to about 250 mg/hour, about 20 mg/hour to about 200 mg/hour, about 20 mg/hour to about 180 mg/hour, about 20 mg/hour to about 160 mg/hour, about 20 mg/hour to about 140 mg/hour, about 20 mg/hour to about 120 mg/hour, about 20 mg/hour to about 100 mg/hour, about 20 mg/hour to about 80 mg/hour, about 20 mg/hour to about 60 mg/hour, about 20 mg/hour to about 50 mg/hour, about 20 mg/hour to about 40 mg/hour, about 40 mg/hour to about 500 mg/hour, about 40 mg/hour to about 450 mg/hour, about 40 mg/hour to about 400 mg/hour, about 40 mg/hour to about 350 mg/hour, about 40 mg/hour to about 300 mg/hour, about 40 mg/hour to about 250 mg/hour, about 40 mg/hour to about 200 mg/hour, about 40 mg/hour to about 180 mg/hour, about 40 mg/hour to about 160 mg/hour, about 40 mg/hour to about 140 mg/hour, about 40 mg/hour to about 120 mg/hour, about 40 mg/hour to about 100 mg/hour, about 40 mg/hour to about 80 mg/hour, about 40 mg/hour to about 60 mg/hour, about 40 mg/hour to about 50 mg/hour, about 50 mg/hour to about 500 mg/hour, about 50 mg/hour to about 450 mg/hour, about 50 mg/hour to about 400 mg/hour, about 50 mg/hour to about 350 mg/hour, about 50 mg/hour to about 300 mg/hour, about 50 mg/hour to about 250 mg/hour, about 50 mg/hour to about 200 mg/hour, about 50 mg/hour to about 180 mg/hour, about 50 mg/hour to about 160 mg/hour, about 50 mg/hour to about 140 mg/hour, about 50 mg/hour to about 120 mg/hour, about 50 mg/hour to about 100 mg/hour, about 50 mg/hour to about 80 mg/hour, about 50 mg/hour to about 60 mg/hour, about 60 mg/hour to about 500 mg/hour, about 60 mg/hour to about 450 mg/hour, about 60 mg/hour to about 400 mg/hour, about 60 mg/hour to about 350 mg/hour, about 60 mg/hour to about 300 mg/hour, about 60 mg/hour to about 250 mg/hour, about 60 mg/hour to about 200 mg/hour, about 60 mg/hour to about 180 mg/hour, about 60 mg/hour to about 160 mg/hour, about 60 mg/hour to about 140 mg/hour, about 60 mg/hour to about 120 mg/hour, about 60 mg/hour to about 100 mg/hour, about 60 mg/hour to about 80 mg/hour, about 80 mg/hour to about 500 mg/hour, about 80 mg/hour to about 450 mg/hour, about 80 mg/hour to about 400 mg/hour, about 80 mg/hour to about 350 mg/hour, about 80 mg/hour to about 300 mg/hour, about 80 mg/hour to about 250 mg/hour, about 80 mg/hour to about 200 mg/hour, about 80 mg/hour to about 180 mg/hour, about 80 mg/hour to about 160 mg/hour, about 80 mg/hour to about 140 mg/hour, about 80 mg/hour to about 120 mg/hour, about 80 mg/hour to about 100 mg/hour, about 100 mg/hour to about 500 mg/hour, about 100 mg/hour to about 450 mg/hour, about 100 mg/hour to about 400 mg/hour, about 100 mg/hour to about 350 mg/hour, about 100 mg/hour to about 300 mg/hour, about 100 mg/hour to about 250 mg/hour, about 100 mg/hour to about 200 mg/hour, about 100 mg/hour to about 180 mg/hour, about 100 mg/hour to about 160 mg/hour, about 100 mg/hour to about 140 mg/hour, about 100 mg/hour to about 120 mg/hour, about 120 mg/hour to about 500 mg/hour, about 120 mg/hour to about 450 mg/hour, about 120 mg/hour to about 400 mg/hour, about 120 mg/hour to about 350 mg/hour, about 120 mg/hour to about 300 mg/hour, about 120 mg/hour to about 250 mg/hour, about 120 mg/hour to about 200 mg/hour, about 120 mg/hour to about 180 mg/hour, about 120 mg/hour to about 160 mg/hour, about 120 mg/hour to about 140 mg/hour, about 140 mg/hour to about 500 mg/hour, about 140 mg/hour to about 450 mg/hour, about 140 mg/hour to about 400 mg/hour, about 140 mg/hour to about 350 mg/hour, about 140 mg/hour to about 300 mg/hour, about 140 mg/hour to about 250 mg/hour, about 140 mg/hour to about 200 mg/hour, about 140 mg/hour to about 180 mg/hour, about 140 mg/hour to about 160 mg/hour, about 160 mg/hour to about 500 mg/hour, about 160 mg/hour to about 450 mg/hour, about 160 mg/hour to about 400 mg/hour, about 160 mg/hour to about 350 mg/hour, about 160 mg/hour to about 300 mg/hour, about 160 mg/hour to about 250 mg/hour, about 160 mg/hour to about 200 mg/hour, about 160 mg/hour to about 180 mg/hour, about 180 mg/hour to about 500 mg/hour, about 180 mg/hour to about 450 mg/hour, about 180 mg/hour to about 400 mg/hour, about 180 mg/hour to about 350 mg/hour, about 180 mg/hour to about 300 mg/hour, about 180 mg/hour to about 250 mg/hour, about 180 mg/hour to about 200 mg/hour, about 200 mg/hour to about 500 mg/hour, about 200 mg/hour to about 450 mg/hour, about 200 mg/hour to about 400 mg/hour, about 200 mg/hour to about 350 mg/hour, about 200 mg/hour to about 300 mg/hour, about 200 mg/hour to about 250 mg/hour, about 250 mg/hour to about 500 mg/hour, about 250 mg/hour to about 450 mg/hour, about 250 mg/hour to about 400 mg/hour, about 250 mg/hour to about 350 mg/hour, about 250 mg/hour to about 300 mg/hour, about 300 mg/hour to about 500 mg/hour, about 300 mg/hour to about 450 mg/hour, about 300 mg/hour to about 400 mg/hour, about 300 mg/hour to about 350 mg/hour, about 350 mg/hour to about 500 mg/hour, about 350 mg/hour to about 450 mg/hour, about 350 mg/hour to about 400 mg/hour, about 400 mg/hour to about 500 mg/hour, about 400 mg/hour to about 450 mg/hour, or about 450 mg/hour to about 500 mg/hour).

In some embodiments, the step-wise infusion rate is increased about every 10 minutes. In some embodiments, the step-wise infusion rate is increased about every 20 minutes. In some embodiments, the step-wise infusion rate is increased about every 30 minutes. In some embodiments, the step-wise infusion rate is increased about every 40 minutes. In some embodiments, the step-wise infusion rate is increased about every 50 minutes. In some embodiments, the step-wise infusion rate is increased about every 60 minutes. In some embodiments, during the step-wise infusion, the infusion rate is increased no more than about two-fold, about every 30 minute.

In some embodiments, administration of a pharmaceutical composition comprising nirogacestat can be oral administration. In such embodiments, the pharmaceutical composition comprising nirogacestat can be formulated as a tablet, a capsule, or aqueous suspension.

### E. Pharmacokinetic Effects

In some embodiments, the administration of the pharmaceutical composition described herein, using any of the methods described herein, results in a steady-state concentration of the antibody or antigen-binding fragment thereof, in the serum of the subject that is able to bind to at least 50%, at least 60%, at least 70%, at least 80%, at least 90%, at least 91%, at least 92%, at least 93%, at least 94%, at least 95%, at least 96%, at least 97%, at least 98%, or at least 99% of the BCMA expressed on the surface of tumor cells in the subject.

In certain embodiments, the antibody or antigen-binding fragment is administered under dose and infusion rates such that the half-life of the antibody or antigen-binding fragment is at least 4, 5, 6, 7, 8, 9, 10, 11, 12, 13, 14 or 15 days. In other embodiments, the half-life is at least one week, at least two weeks, at least three weeks, or at least four weeks.

Some embodiments of these methods result in a steady-state concentration of the antibody, or antigen-binding fragment thereof, in the serum of the subject of about 1 μg/mL to about 200 μg/mL (e.g., about 1 μg/mL to about 180 μg/mL, about 1 μg/mL to about 160 μg/mL, about 1 μg/mL to about 140 μg/mL, about 1 μg/mL to about 120 μg/mL, about 1 μg/mL to about 100 μg/mL, about 1 μg/mL to about 90 μg/mL, about 1 μg/mL to about 80 μg/mL, about 1 μg/mL to about 70 μg/mL, about 1 μg/mL to about 60 μg/mL, about 1 μg/mL to about 50 μg/mL, about 1 μg/mL to about 40 μg/mL, about 1 μg/mL to about 30 μg/mL, about 1 μg/mL to about 20 μg/mL, about 1 μg/mL to about 10 μg/mL, about 10 μg/mL to about 200 μg/mL, about 10 μg/mL to about 180 μg/mL, about 10 μg/mL to about 160 μg/mL, about 10 μg/mL to about 140 μg/mL, about 10 μg/mL to about 120 μg/mL, about 10 μg/mL to about 100 μg/mL, about 10 μg/mL to about 90 μg/mL, about 10 μg/mL to about 80 μg/mL, about 10 μg/mL to about 70 μg/mL, about 10 μg/mL to about 60 μg/mL, about 10 μg/mL to about 50 μg/mL, about 10 μg/mL to about 40 μg/mL, about 10 μg/mL to about 30 μg/mL, about 10 μg/mL to about 20 μg/mL, about 20 μg/mL to about 200 μg/mL, about 20 μg/mL to about 180 μg/mL, about 20 μg/mL to about 160 μg/mL, about 20 μg/mL to about 140 μg/mL, about 20 μg/mL to about 120 μg/mL, about 20 μg/mL to about 100 μg/mL, about 20 μg/mL to about 90 μg/mL, about 20 μg/mL to about 80 μg/mL, about 20 μg/mL to about 70 μg/mL, about 20 μg/mL to about 60 μg/mL, about 20 μg/mL to about 50 μg/mL, about 20 μg/mL to about 40 μg/mL, about 20 μg/mL to about 30 μg/mL, about 30 μg/mL to about 200 μg/mL, about 30 μg/mL to about 180 μg/mL, about 30 μg/mL to about 160 μg/mL, about 30 μg/mL to about 140 μg/mL, about 30 μg/mL to about 120 μg/mL, about 30 μg/mL to about 100 μg/mL, about 30 μg/mL to about 90 μg/mL, about 30 μg/mL to about 80 μg/mL, about 30 μg/mL to about 70 μg/mL, about 30 μg/mL to about 60 μg/mL, about 30 μg/mL to about 50 μg/mL, about 30 μg/mL to about 40 μg/mL, about 40 μg/mL to about 200 μg/mL, about 40 μg/mL to about 180 μg/mL, about 40 μg/mL to about 160 μg/mL, about 40 μg/mL to about 140 μg/mL, about 40 μg/mL to about 120 μg/mL, about 40 μg/mL to about 100 μg/mL, about 40 μg/mL to about 90 μg/mL, about 40 μg/mL to about 80 μg/mL, about 40 μg/mL to about 70 μg/mL, about 40 μg/mL to about 60 μg/mL, about 40 μg/mL to about 50 μg/mL, about 50 μg/mL to about 200 μg/mL, about 50 μg/mL to about 180 μg/mL, about 50 μg/mL to about 160 μg/mL, about 50 μg/mL to about 140 μg/mL, about 50 μg/mL to about 120 μg/mL, about 50 μg/mL to about 100 μg/mL, about 50 μg/mL to about 90 μg/mL, about 50 μg/mL to about 80 μg/mL, about 50 μg/mL to about 70 μg/mL, about 50 μg/mL to about 60 μg/mL, about 60 μg/mL to about 200 μg/mL, about 60 μg/mL to about 180 μg/mL, about 60 μg/mL to about 160 μg/mL, about 60 μg/mL to about 140 μg/mL, about 60 μg/mL to about 120 μg/mL, about 60 μg/mL to about 100 μg/mL, about 60 μg/mL to about 90 μg/mL, about 60 μg/mL to about 80 μg/mL, about 60 μg/mL to about 70 μg/mL, about 70 μg/mL to about 200 μg/mL, about 70 μg/mL to about 180 μg/mL, about 70 μg/mL to about 160 μg/mL, about 70 μg/mL to about 140 μg/mL, about 70 μg/mL to about 120 μg/mL, about 70 μg/mL to about 100 μg/mL, about 70 μg/mL to about 90 μg/mL, about 70 μg/mL to about 80 μg/mL, about 80 μg/mL to about 200 μg/mL, about 80 μg/mL to about 180 μg/mL, about 80 μg/mL to about 160 μg/mL, about 80 μg/mL to about 140 μg/mL, about 80 μg/mL to about 120 μg/mL, about 80 μg/mL to about 100 μg/mL, about 80 μg/mL to about 90 μg/mL, about 90 μg/mL to about 200 μg/mL, about 90 μg/mL to about 180 μg/mL, about 90 μg/mL to about 160 μg/mL, about 90 μg/mL to about 140 μg/mL, about 90 μg/mL to about 120 μg/mL, about 90 μg/mL to about 100 μg/mL, about 100 μg/mL to about 200 μg/mL, about 100 μg/mL to about 180 μg/mL, about 100 μg/mL to about 160 μg/mL, about 100 μg/mL to about 140 μg/mL, about 100 μg/mL to about 120 μg/mL, about 120 μg/mL to about 200 μg/mL, about 120 μg/mL to about 180 μg/mL, about 120 μg/mL to about 160 μg/mL, about 120 μg/mL to about 140 μg/mL, about 140 μg/mL to about 200 μg/mL, about 140 μg/mL to about 180 μg/mL, about 140 μg/mL to about 160 μg/mL, about 160 μg/mL to about 200 μg/mL, about 160 μg/mL to about 180 μg/mL, or about 180 μg/mL to about 200 μg/mL) (e.g., for about 6 hours to about one year (e.g., about 6 hours to about 11.5 months, about 6 hours to about 11.0 months, about 6 hours to about 10.5 months, about 6 hours to about 10.0 months, about 6 hours to about 9.5 months, about 6 hours to about 9.0 months, about 6 hours to about 8.5 months, about 6 hours to about 8.0 months, about 6 hours to about 7.5 months, about 6 hours to about 7.0 months, about 6 hours to about 6.5 months, about 6 hours to about 6.0 months, about 6 hours to about 5.5 months, about 6 hours to about 5.0 months, about 6 hours to about 4.5 months, about 6 hours to about 4.0 months, about 6 hours to about 3.5 months, about 6 hours to about 3.0 months, about 6 hours to about 2.5 months, about 6 hours to about 2.0 months, about 6 hours to about 1.5 months, about 6 hours to about 5 weeks, about 6 hours to about 4 weeks, about 6 hours to about 3 weeks, about 6 hours to about 2 weeks, about 6 hours to about 1 week, about 6 hours to about 5 days, about 6 hours to about 3 days, about 6 hours to about 1 day, about 6 hours to about 18 hours, about 6 hours to about 12 hours, about 12 hours to about 1 year, about 12 hours to about 11.5 months, about 12 hours to about 11.0 months, about 12 hours to about 10.5 months, about 12 hours to about 10.0 months, about 12 hours to about 9.5 months, about 12 hours to about 9.0 months, about 12 hours to about 8.5 months, about 12 hours to about 8.0 months, about 12 hours to about 7.5 months, about 12 hours to about 7.0 months, about 12 hours to about 6.5 months, about 12 hours to about 6.0 months, about 12 hours to about 5.5 months, about 12 hours to about 5.0 months, about 12 hours to about 4.5 months, about 12 hours to about 4.0 months, about 12 hours to about 3.5 months, about 12 hours to about 3.0 months, about 12 hours to about 2.5 months, about 12 hours to about 2.0 months, about 12 hours to about 1.5 months, about 12 hours to about 5 weeks, about 12 hours to about 4 weeks, about 12 hours to about 3 weeks, about 12 hours to about 2 weeks, about 12 hours to about 1 week, about 12 hours to about 5 days, about 12 hours to about 3 days, about 12 hours to about 1 day, about 12 hours to about 18 hours, about 18 hours to about 1 year, about 18 hours to about 11.5 months, about 18 hours to about 11.0 months, about 18 hours to about 10.5 months, about 18 hours to about 10.0 months, about 18 hours to about 9.5 months, about 18 hours to about 9.0 months, about 18 hours to about 8.5 months, about 18 hours to about 8.0 months, about 18 hours to about 7.5 months, about 18 hours to about 7.0 months, about 18 hours to about 6.5 months, about 18 hours to about 6.0 months, about 18 hours to about 5.5 months, about 18 hours to about 5.0 months, about 18 hours to about 4.5 months, about 18 hours to about 4.0 months, about 18 hours to about 3.5 months, about 18 hours to about 3.0 months, about 18 hours to about 2.5 months, about 18 hours to about 2.0 months, about 18 hours to about 1.5 months, about 18 hours to about 5 weeks, about 18 hours to about 4 weeks, about 18 hours to about 3 weeks, about 18 hours to about 2 weeks, about 18 hours to about 1 week, about 18 hours to about 5 days, about 18 hours to about 3 days, about 18 hours to about 1 day, about 1 day to about 1 year, about 1 day to about 11.5 months, about 1 day to about 11.0 months, about 1 day to about 10.5 months, about 1 day to about 10.0 months, about 1 day to about 9.5 months, about 1 day to about 9.0 months, about 1 day to about 8.5 months, about 1 day to about 8.0 months, about 1 day to about 7.5 months, about 1 day to about 7.0 months, about 1 day to about 6.5 months, about 1 day to about 6.0 months, about 1 day to about 5.5 months, about 1 day to about 5.0 months, about 1 day to about 4.5 months, about 1 day to about 4.0 months, about 1 day to about 3.5 months, about 1 day to about 3.0 months, about 1 day to about 2.5 months, about 1 day to about 2.0 months, about 1 day to about 1.5 months, about 1 day to about 5 weeks, about 1 day to about 4 weeks, about 1 day to about 3 weeks, about 1 day to about 2 weeks, about 1 day to about 1 week, about 1 day to about 5 days, about 1 day to about 3 days, about 3 days to about 1 year, about 3 days to about 11.5 months, about 3 days to about 11.0 months, about 3 days to about 10.5 months, about 3 days to about 10.0 months, about 3 days to about 9.5 months, about 3 days to about 9.0 months, about 3 days to about 8.5 months, about 3 days to about 8.0 months, about 3 days to about 7.5 months, about 3 days to about 7.0 months, about 3 days to about 6.5 months, about 3 days to about 6.0 months, about 3 days to about 5.5 months, about 3 days to about 5.0 months, about 3 days to about 4.5 months, about 3 days to about 4.0 months, about 3 days to about 3.5 months, about 3 days to about 3.0 months, about 3 days to about 2.5 months, about 3 days to about 2.0 months, about 3 days to about 1.5 months, about 3 days to about 5 weeks, about 3 days to about 4 weeks, about 3 days to about 3 weeks, about 3 days to about 2 weeks, about 3 days to about 1 week, about 3 days to about 5 days, about 5 days to about 1 year, about 5 days to about 11.5 months, about 5 days to about 11.0 months, about 5 days to about 10.5 months, about 5 days to about 10.0 months, about 5 days to about 9.5 months, about 5 days to about 9.0 months, about 5 days to about 8.5 months, about 5 days to about 8.0 months, about 5 days to about 7.5 months, about 5 days to about 7.0 months, about 5 days to about 6.5 months, about 5 days to about 6.0 months, about 5 days to about 5.5 months, about 5 days to about 5.0 months, about 5 days to about 4.5 months, about 5 days to about 4.0 months, about 5 days to about 3.5 months, about 5 days to about 3.0 months, about 5 days to about 2.5 months, about 5 days to about 2.0 months, about 5 days to about 1.5 months, about 5 days to about 5 weeks, about 5 days to about 4 weeks, about 5 days to about 3 weeks, about 5 days to about 2 weeks, about 5 days to about 1 week, about 1 week to about 1 year, about 1 week to about 11.5 months, about 1 week to about 11.0 months, about 1 week to about 10.5 months, about 1 week to about 10.0 months, about 1 week to about 9.5 months, about 1 week to about 9.0 months, about 1 week to about 8.5 months, about 1 week to about 8.0 months, about 1 week to about 7.5 months, about 1 week to about 7.0 months, about 1 week to about 6.5 months, about 1 week to about 6.0 months, about 1 week to about 5.5 months, about 1 week to about 5.0 months, about 1 week to about 4.5 months, about 1 week to about 4.0 months, about 1 week to about 3.5 months, about 1 week to about 3.0 months, about 1 week to about 2.5 months, about 1 week to about 2.0 months, about 1 week to about 1.5 months, about 1 week to about 5 weeks, about 1 week to about 4 weeks, about 1 week to about 3 weeks, about 1 week to about 2 weeks, about 2 weeks to about 1 year, about 2 weeks to about 11.5 months, about 2 weeks to about 11.0 months, about 2 weeks to about 10.5 months, about 2 weeks to about 10.0 months, about 2 weeks to about 9.5 months, about 2 weeks to about 9.0 months, about 2 weeks to about 8.5 months, about 2 weeks to about 8.0 months, about 2 weeks to about 7.5 months, about 2 weeks to about 7.0 months, about 2 weeks to about 6.5 months, about 2 weeks to about 6.0 months, about 2 weeks to about 5.5 months, about 2 weeks to about 5.0 months, about 2 weeks to about 4.5 months, about 2 weeks to about 4.0 months, about 2 weeks to about 3.5 months, about 2 weeks to about 3.0 months, about 2 weeks to about 2.5 months, about 2 weeks to about 2.0 months, about 2 weeks to about 1.5 months, about 2 weeks to about 5 weeks, about 2 weeks to about 4 weeks, about 2 weeks to about 3 weeks, about 3 weeks to about 1 year, about 3 weeks to about 11.5 months, about 3 weeks to about 11.0 months, about 3 weeks to about 10.5 months, about 3 weeks to about 10.0 months, about 3 weeks to about 9.5 months, about 3 weeks to about 9.0 months, about 3 weeks to about 8.5 months, about 3 weeks to about 8.0 months, about 3 weeks to about 7.5 months, about 3 weeks to about 7.0 months, about 3 weeks to about 6.5 months, about 3 weeks to about 6.0 months, about 3 weeks to about 5.5 months, about 3 weeks to about 5.0 months, about 3 weeks to about 4.5 months, about 3 weeks to about 4.0 months, about 3 weeks to about 3.5 months, about 3 weeks to about 3.0 months, about 3 weeks to about 2.5 months, about 3 weeks to about 2.0 months, about 3 weeks to about 1.5 months, about 3 weeks to about 5 weeks, about 3 weeks to about 4 weeks, about 4 weeks to about 1 year, about 4 weeks to about 11.5 months, about 4 weeks to about 11.0 months, about 4 weeks to about 10.5 months, about 4 weeks to about 10.0 months, about 4 weeks to about 9.5 months, about 4 weeks to about 9.0 months, about 4 weeks to about 8.5 months, about 4 weeks to about 8.0 months, about 4 weeks to about 7.5 months, about 4 weeks to about 7.0 months, about 4 weeks to about 6.5 months, about 4 weeks to about 6.0 months, about 4 weeks to about 5.5 months, about 4 weeks to about 5.0 months, about 4 weeks to about 4.5 months, about 4 weeks to about 4.0 months, about 4 weeks to about 3.5 months, about 4 weeks to about 3.0 months, about 4 weeks to about 2.5 months, about 4 weeks to about 2.0 months, about 4 weeks to about 1.5 months, about 4 weeks to about 5 weeks, about 5 weeks to about 1 year, about 5 weeks to about 11.5 months, about 5 weeks to about 11.0 months, about 5 weeks to about 10.5 months, about 5 weeks to about 10.0 months, about 5 weeks to about 9.5 months, about 5 weeks to about 9.0 months, about 5 weeks to about 8.5 months, about 5 weeks to about 8.0 months, about 5 weeks to about 7.5 months, about 5 weeks to about 7.0 months, about 5 weeks to about 6.5 months, about 5 weeks to about 6.0 months, about 5 weeks to about 5.5 months, about 5 weeks to about 5.0 months, about 5 weeks to about 4.5 months, about 5 weeks to about 4.0 months, about 5 weeks to about 3.5 months, about 5 weeks to about 3.0 months, about 5 weeks to about 2.5 months, about 5 weeks to about 2.0 months, about 5 weeks to about 1.5 months, about 1.5 months to about 1 year, about 1.5 months to about 11.5 months, about 1.5 months to about 11.0 months, about 1.5 months to about 10.5 months, about 1.5 months to about 10.0 months, about 1.5 months to about 9.5 months, about 1.5 months to about 9.0 months, about 1.5 months to about 8.5 months, about 1.5 months to about 8.0 months, about 1.5 months to about 7.5 months, about 1.5 months to about 7.0 months, about 1.5 months to about 6.5 months, about 1.5 months to about 6.0 months, about 1.5 months to about 5.5 months, about 1.5 months to about 5.0 months, about 1.5 months to about 4.5 months, about 1.5 months to about 4.0 months, about 1.5 months to about 3.5 months, about 1.5 months to about 3.0 months, about 1.5 months to about 2.5 months, about 1.5 months to about 2.0 months, about 2.0 months to about 1 year, about 2.0 months to about 11.5 months, about 2.0 months to about 11.0 months, about 2.0 months to about 10.5 months, about 2.0 months to about 10.0 months, about 2.0 months to about 9.5 months, about 2.0 months to about 9.0 months, about 2.0 months to about 8.5 months, about 2.0 months to about 8.0 months, about 2.0 months to about 7.5 months, about 2.0 months to about 7.0 months, about 2.0 months to about 6.5 months, about 2.0 months to about 6.0 months, about 2.0 months to about 5.5 months, about 2.0 months to about 5.0 months, about 2.0 months to about 4.5 months, about 2.0 months to about 4.0 months, about 2.0 months to about 3.5 months, about 2.0 months to about 3.0 months, about 2.0 months to about 2.5 months, about 2.5 months to about 1 year, about 2.5 months to about 11.5 months, about 2.5 months to about 11.0 months, about 2.5 months to about 10.5 months, about 2.5 months to about 10.0 months, about 2.5 months to about 9.5 months, about 2.5 months to about 9.0 months, about 2.5 months to about 8.5 months, about 2.5 months to about 8.0 months, about 2.5 months to about 7.5 months, about 2.5 months to about 7.0 months, about 2.5 months to about 6.5 months, about 2.5 months to about 6.0 months, about 2.5 months to about 5.5 months, about 2.5 months to about 5.0 months, about 2.5 months to about 4.5 months, about 2.5 months to about 4.0 months, about 2.5 months to about 3.5 months, about 2.5 months to about 3.0 months, about 3.0 months to about 1 year, about 3.0 months to about 11.5 months, about 3.0 months to about 11.0 months, about 3.0 months to about 10.5 months, about 3.0 months to about 10.0 months, about 3.0 months to about 9.5 months, about 3.0 months to about 9.0 months, about 3.0 months to about 8.5 months, about 3.0 months to about 8.0 months, about 3.0 months to about 7.5 months, about 3.0 months to about 7.0 months, about 3.0 months to about 6.5 months, about 3.0 months to about 6.0 months, about 3.0 months to about 5.5 months, about 3.0 months to about 5.0 months, about 3.0 months to about 4.5 months, about 3.0 months to about 4.0 months, about 3.0 months to about 3.5 months, about 3.5 months to about 1 year, about 3.5 months to about 11.5 months, about 3.5 months to about 11.0 months, about 3.5 months to about 10.5 months, about 3.5 months to about 10.0 months, about 3.5 months to about 9.5 months, about 3.5 months to about 9.0 months, about 3.5 months to about 8.5 months, about 3.5 months to about 8.0 months, about 3.5 months to about 7.5 months, about 3.5 months to about 7.0 months, about 3.5 months to about 6.5 months, about 3.5 months to about 6.0 months, about 3.5 months to about 5.5 months, about 3.5 months to about 5.0 months, about 3.5 months to about 4.5 months, about 3.5 months to about 4.0 months, about 4.0 months to about 1 year, about 4.0 months to about 11.5 months, about 4.0 months to about 11.0 months, about 4.0 months to about 10.5 months, about 4.0 months to about 10.0 months, about 4.0 months to about 9.5 months, about 4.0 months to about 9.0 months, about 4.0 months to about 8.5 months, about 4.0 months to about 8.0 months, about 4.0 months to about 7.5 months, about 4.0 months to about 7.0 months, about 4.0 months to about 6.5 months, about 4.0 months to about 6.0 months, about 4.0 months to about 5.5 months, about 4.0 months to about 5.0 months, about 4.0 months to about 4.5 months, about 4.5 months to about 1 year, about 4.5 months to about 11.5 months, about 4.5 months to about 11.0 months, about 4.5 months to about 10.5 months, about 4.5 months to about 10.0 months, about 4.5 months to about 9.5 months, about 4.5 months to about 9.0 months, about 4.5 months to about 8.5 months, about 4.5 months to about 8.0 months, about 4.5 months to about 7.5 months, about 4.5 months to about 7.0 months, about 4.5 months to about 6.5 months, about 4.5 months to about 6.0 months, about 4.5 months to about 5.5 months, about 4.5 months to about 5.0 months, about 5.0 months to about 1 year, about 5.0 months to about 11.5 months, about 5.0 months to about 11.0 months, about 5.0 months to about 10.5 months, about 5.0 months to about 10.0 months, about 5.0 months to about 9.5 months, about 5.0 months to about 9.0 months, about 5.0 months to about 8.5 months, about 5.0 months to about 8.0 months, about 5.0 months to about 7.5 months, about 5.0 months to about 7.0 months, about 5.0 months to about 6.5 months, about 5.0 months to about 6.0 months, about 5.0 months to about 5.5 months, about 5.5 months to about 1 year, about 5.5 months to about 11.5 months, about 5.5 months to about 11.0 months, about 5.5 months to about 10.5 months, about 5.5 months to about 10.0 months, about 5.5 months to about 9.5 months, about 5.5 months to about 9.0 months, about 5.5 months to about 8.5 months, about 5.5 months to about 8.0 months, about 5.5 months to about 7.5 months, about 5.5 months to about 7.0 months, about 5.5 months to about 6.5 months, about 5.5 months to about 6.0 months, about 6.0 months to about 1 year, about 6.0 months to about 11.5 months, about 6.0 months to about 11.0 months, about 6.0 months to about 10.5 months, about 6.0 months to about 10.0 months, about 6.0 months to about 9.5 months, about 6.0 months to about 9.0 months, about 6.0 months to about 8.5 months, about 6.0 months to about 8.0 months, about 6.0 months to about 7.5 months, about 6.0 months to about 7.0 months, about 6.0 months to about 6.5 months, about 6.5 months to about 1 year, about 6.5 months to about 11.5 months, about 6.5 months to about 11.0 months, about 6.5 months to about 10.5 months, about 6.5 months to about 10.0 months, about 6.5 months to about 9.5 months, about 6.5 months to about 9.0 months, about 6.5 months to about 8.5 months, about 6.5 months to about 8.0 months, about 6.5 months to about 7.5 months, about 6.5 months to about 7.0 months, about 7.0 months to about 1 year, about 7.0 months to about 11.5 months, about 7.0 months to about 11.0 months, about 7.0 months to about 10.5 months, about 7.0 months to about 10.0 months, about 7.0 months to about 9.5 months, about 7.0 months to about 9.0 months, about 7.0 months to about 8.5 months, about 7.0 months to about 8.0 months, about 7.0 months to about 7.5 months, about 7.5 months to about 1 year, about 7.5 months to about 11.5 months, about 7.5 months to about 11.0 months, about 7.5 months to about 10.5 months, about 7.5 months to about 10.0 months, about 7.5 months to about 9.5 months, about 7.5 months to about 9.0 months, about 7.5 months to about 8.5 months, about 7.5 months to about 8.0 months, about 8.0 months to about 1 year, about 8.0 months to about 11.5 months, about 8.0 months to about 11.0 months, about 8.0 months to about 10.5 months, about 8.0 months to about 10.0 months, about 8.0 months to about 9.5 months, about 8.0 months to about 9.0 months, about 8.0 months to about 8.5 months, about 8.5 months to about 1 year, about 8.5 months to about 11.5 months, about 8.5 months to about 11.0 months, about 8.5 months to about 10.5 months, about 8.5 months to about 10.0 months, about 8.5 months to about 9.5 months, about 8.5 months to about 9.0 months, about 9.0 months to about 1 year, about 9.0 months to about 11.5 months, about 9.0 months to about 11.0 months, about 9.0 months to about 10.5 months, about 9.0 months to about 10.0 months, about 9.0 months to about 9.5 months, about 9.5 months to about 1 year, about 9.5 months to about 11.5 months, about 9.5 months to about 11.0 months, about 9.5 months to about 10.5 months, about 9.5 months to about 10.0 months, about 10.0 months to about 1 year, about 10.0 months to about 11.5 months, about 10.0 months to about 11.0 months, about 10.0 months to about 10.5 months, about 10.5 months to about 1 year, about 10.5 months to about 11.5 months, about 10.5 months to about 11.0 months, about 11.0 months to about 1 year, about 11.0 months to about 11.5 months, or about 11.5 months to about 1 year) after the administration of a first dose of the antibody or the antigen-binding fragment.

Some embodiments of these methods result in a steady-state concentration of free light chain (FLC), in the serum of the subject of less than about 50 mg/dL, less than about 45 mg/dL, less than about 40 mg/dL, less than about 35 mg/dL, less than about 30 mg/dL, less than about 25 mg/dL, less than about 20 mg/dL, less than about 18 mg/dL, less than about 16 mg/dL, less than about 14 mg/dL, less than about 12 mg/dL, less than about 10 mg/dL, less than about 8 mg/dL, less than about 6 mg/dL, less than about 4 mg/dL, less than about 2 mg/dL, or less than about 1 mg/dL (e.g., for about 6 hours to about one year, or any of the subranges of this range, after the administration of a first dose of the antibody or the antigen-binding fragment and a first dose of nirogacestat to the subject).

Some embodiments of these methods result in a steady-state concentration of free light chain (FLC), in the serum of the subject of about 0.1 mg/dL to about 50 mg/dL (e.g., about 0.1 mg/dL to about 48 mg/dL, about 0.1 mg/dL to about 45 mg/dL, about 0.1 mg/dL to about 40 mg/dL, about 0.1 mg/dL to about 35 mg/dL, about 0.1 mg/dL to about 30 mg/dL, about 0.1 mg/dL to about 25 mg/dL, about 0.1 mg/dL to about 20 mg/dL, about 0.1 mg/dL to about 18 mg/dL, about 0.1 mg/dL to about 16 mg/dL, about 0.1 mg/dL to about 14 mg/dL, about 0.1 mg/dL to about 12 mg/dL, about 0.1 mg/dL to about 10 mg/dL, about 0.1 mg/dL to about 8 mg/dL, about 0.1 mg/dL to about 6 mg/dL, about 0.1 mg/dL to about 4 mg/dL, about 0.1 mg/dL to about 2 mg/dL, about 0.1 mg/dL to about 1.0 mg/dL, about 0.1 mg/dL to about 0.5 mg/dL, about 0.1 mg/dL to about 0.2 mg/dL, about 0.2 mg/dL to about 50 mg/dL, about 0.2 mg/dL to about 48 mg/dL, about 0.2 mg/dL to about 45 mg/dL, about 0.2 mg/dL to about 40 mg/dL, about 0.2 mg/dL to about 35 mg/dL, about 0.2 mg/dL to about 30 mg/dL, about 0.2 mg/dL to about 25 mg/dL, about 0.2 mg/dL to about 20 mg/dL, about 0.2 mg/dL to about 18 mg/dL, about 0.2 mg/dL to about 16 mg/dL, about 0.2 mg/dL to about 14 mg/dL, about 0.2 mg/dL to about 12 mg/dL, about 0.2 mg/dL to about 10 mg/dL, about 0.2 mg/dL to about 8 mg/dL, about 0.2 mg/dL to about 6 mg/dL, about 0.2 mg/dL to about 4 mg/dL, about 0.2 mg/dL to about 2 mg/dL, about 0.2 mg/dL to about 1.0 mg/dL, about 0.2 mg/dL to about 0.5 mg/dL, about 0.5 mg/dL to about 50 mg/dL, about 0.5 mg/dL to about 48 mg/dL, about 0.5 mg/dL to about 45 mg/dL, about 0.5 mg/dL to about 40 mg/dL, about 0.5 mg/dL to about 35 mg/dL, about 0.5 mg/dL to about 30 mg/dL, about 0.5 mg/dL to about 25 mg/dL, about 0.5 mg/dL to about 20 mg/dL, about 0.5 mg/dL to about 18 mg/dL, about 0.5 mg/dL to about 16 mg/dL, about 0.5 mg/dL to about 14 mg/dL, about 0.5 mg/dL to about 12 mg/dL, about 0.5 mg/dL to about 10 mg/dL, about 0.5 mg/dL to about 8 mg/dL, about 0.5 mg/dL to about 6 mg/dL, about 0.5 mg/dL to about 4 mg/dL, about 0.5 mg/dL to about 2 mg/dL, about 0.5 mg/dL to about 1.0 mg/dL, about 1.0 mg/dL to about 50 mg/dL, about 1.0 mg/dL to about 48 mg/dL, about 1.0 mg/dL to about 45 mg/dL, about 1.0 mg/dL to about 40 mg/dL, about 1.0 mg/dL to about 35 mg/dL, about 1.0 mg/dL to about 30 mg/dL, about 1.0 mg/dL to about 25 mg/dL, about 1.0 mg/dL to about 20 mg/dL, about 1.0 mg/dL to about 18 mg/dL, about 1.0 mg/dL to about 16 mg/dL, about 1.0 mg/dL to about 14 mg/dL, about 1.0 mg/dL to about 12 mg/dL, about 1.0 mg/dL to about 10 mg/dL, about 1.0 mg/dL to about 8 mg/dL, about 1.0 mg/dL to about 6 mg/dL, about 1.0 mg/dL to about 4 mg/dL, about 1.0 mg/dL to about 2 mg/dL, about 2 mg/dL to about 50 mg/dL, about 2 mg/dL to about 48 mg/dL, about 2 mg/dL to about 45 mg/dL, about 2 mg/dL to about 40 mg/dL, about 2 mg/dL to about 35 mg/dL, about 2 mg/dL to about 30 mg/dL, about 2 mg/dL to about 25 mg/dL, about 2 mg/dL to about 20 mg/dL, about 2 mg/dL to about 18 mg/dL, about 2 mg/dL to about 16 mg/dL, about 2 mg/dL to about 14 mg/dL, about 2 mg/dL to about 12 mg/dL, about 2 mg/dL to about 10 mg/dL, about 2 mg/dL to about 8 mg/dL, about 2 mg/dL to about 6 mg/dL, about 2 mg/dL to about 4 mg/dL, about 4 mg/dL to about 50 mg/dL, about 4 mg/dL to about 48 mg/dL, about 4 mg/dL to about 45 mg/dL, about 4 mg/dL to about 40 mg/dL, about 4 mg/dL to about 35 mg/dL, about 4 mg/dL to about 30 mg/dL, about 4 mg/dL to about 25 mg/dL, about 4 mg/dL to about 20 mg/dL, about 4 mg/dL to about 18 mg/dL, about 4 mg/dL to about 16 mg/dL, about 4 mg/dL to about 14 mg/dL, about 4 mg/dL to about 12 mg/dL, about 4 mg/dL to about 10 mg/dL, about 4 mg/dL to about 8 mg/dL, about 4 mg/dL to about 6 mg/dL, about 6 mg/dL to about 50 mg/dL, about 6 mg/dL to about 48 mg/dL, about 6 mg/dL to about 45 mg/dL, about 6 mg/dL to about 40 mg/dL, about 6 mg/dL to about 35 mg/dL, about 6 mg/dL to about 30 mg/dL, about 6 mg/dL to about 25 mg/dL, about 6 mg/dL to about 20 mg/dL, about 6 mg/dL to about 18 mg/dL, about 6 mg/dL to about 16 mg/dL, about 6 mg/dL to about 14 mg/dL, about 6 mg/dL to about 12 mg/dL, about 6 mg/dL to about 10 mg/dL, about 6 mg/dL to about 8 mg/dL, about 8 mg/dL to about 50 mg/dL, about 8 mg/dL to about 48 mg/dL, about 8 mg/dL to about 45 mg/dL, about 8 mg/dL to about 40 mg/dL, about 8 mg/dL to about 35 mg/dL, about 8 mg/dL to about 30 mg/dL, about 8 mg/dL to about 25 mg/dL, about 8 mg/dL to about 20 mg/dL, about 8 mg/dL to about 18 mg/dL, about 8 mg/dL to about 16 mg/dL, about 8 mg/dL to about 14 mg/dL, about 8 mg/dL to about 12 mg/dL, about 8 mg/dL to about 10 mg/dL, about 10 mg/dL to about 50 mg/dL, about 10 mg/dL to about 48 mg/dL, about 10 mg/dL to about 45 mg/dL, about 10 mg/dL to about 40 mg/dL, about 10 mg/dL to about 35 mg/dL, about 10 mg/dL to about 30 mg/dL, about 10 mg/dL to about 25 mg/dL, about 10 mg/dL to about 20 mg/dL, about 10 mg/dL to about 18 mg/dL, about 10 mg/dL to about 16 mg/dL, about 10 mg/dL to about 14 mg/dL, about 10 mg/dL to about 12 mg/dL, about 12 mg/dL to about 50 mg/dL, about 12 mg/dL to about 48 mg/dL, about 12 mg/dL to about 45 mg/dL, about 12 mg/dL to about 40 mg/dL, about 12 mg/dL to about 35 mg/dL, about 12 mg/dL to about 30 mg/dL, about 12 mg/dL to about 25 mg/dL, about 12 mg/dL to about 20 mg/dL, about 12 mg/dL to about 18 mg/dL, about 12 mg/dL to about 16 mg/dL, about 12 mg/dL to about 14 mg/dL, about 14 mg/dL to about 50 mg/dL, about 14 mg/dL to about 48 mg/dL, about 14 mg/dL to about 45 mg/dL, about 14 mg/dL to about 40 mg/dL, about 14 mg/dL to about 35 mg/dL, about 14 mg/dL to about 30 mg/dL, about 14 mg/dL to about 25 mg/dL, about 14 mg/dL to about 20 mg/dL, about 14 mg/dL to about 18 mg/dL, about 14 mg/dL to about 16 mg/dL, about 16 mg/dL to about 50 mg/dL, about 16 mg/dL to about 48 mg/dL, about 16 mg/dL to about 45 mg/dL, about 16 mg/dL to about 40 mg/dL, about 16 mg/dL to about 35 mg/dL, about 16 mg/dL to about 30 mg/dL, about 16 mg/dL to about 25 mg/dL, about 16 mg/dL to about 20 mg/dL, about 16 mg/dL to about 18 mg/dL, about 18 mg/dL to about 50 mg/dL, about 18 mg/dL to about 48 mg/dL, about 18 mg/dL to about 45 mg/dL, about 18 mg/dL to about 40 mg/dL, about 18 mg/dL to about 35 mg/dL, about 18 mg/dL to about 30 mg/dL, about 18 mg/dL to about 25 mg/dL, about 18 mg/dL to about 20 mg/dL, about 20 mg/dL to about 50 mg/dL, about 20 mg/dL to about 48 mg/dL, about 20 mg/dL to about 45 mg/dL, about 20 mg/dL to about 40 mg/dL, about 20 mg/dL to about 35 mg/dL, about 20 mg/dL to about 30 mg/dL, about 20 mg/dL to about 25 mg/dL, about 25 mg/dL to about 50 mg/dL, about 25 mg/dL to about 48 mg/dL, about 25 mg/dL to about 45 mg/dL, about 25 mg/dL to about 40 mg/dL, about 25 mg/dL to about 35 mg/dL, about 25 mg/dL to about 30 mg/dL, about 30 mg/dL to about 50 mg/dL, about 30 mg/dL to about 48 mg/dL, about 30 mg/dL to about 45 mg/dL, about 30 mg/dL to about 40 mg/dL, about 30 mg/dL to about 35 mg/dL, about 35 mg/dL to about 50 mg/dL, about 35 mg/dL to about 48 mg/dL, about 35 mg/dL to about 45 mg/dL, about 35 mg/dL to about 40 mg/dL, about 40 mg/dL to about 50 mg/dL, about 40 mg/dL to about 48 mg/dL, about 40 mg/dL to about 45 mg/dL, about 45 mg/dL to about 50 mg/dL, about 45 mg/dL to about 48 mg/dL, or about 48 mg/dL to about 50 mg/dL) (e.g., for about 6 hours to about one year, or any of the subranges of this range, after the administration of a first dose of the antibody or the antigen-binding fragment and a first dose of nirogacestat to the subject).

### G. Therapeutic Effects

The therapeutic effects of the methods described herein can be assessed by the expression levels of one or more biomarkers in a patient sample. Exemplary biomarker assessments include testing the levels of serum free light chain and modified serum protein electrophoresis tests (SPEP), peripheral blood immunophenotyping, such as flow cytometry measurements included, but not be limited to, characterizing NK cells, monocytes, T cells, and B cells, assessment of levels of circulating soluble BCMA (sBCMA), a proliferation-inducing ligand (APRIL) and B-cell activation factor (BAFF), retrospective analyses of cellular and circulating biomarkers, characterization of tumor tissue, bone marrow immunotyping, baseline and treatment-related changes in gene expression profiles in tumor and tumor microenvironment assessed by RNA sequencing in tumor and non-tumor cells, and assessment of levels of soluble target, ligands, and/or cytokines/chemokines in bone marrow plasma.

The therapeutic effects achieved by the methods described herein can also include, for example, a decrease in severity of disease symptoms, an increase in frequency and duration of disease symptom-free periods, an increase in lifespan, disease remission, or a prevention of impairment or disability due to the disease affliction. For example, for the treatment of multiple myeloma, aggressive and/or drug resistant and/or refractory multiple myeloma, the methods described herein inhibits cell growth or tumor growth by at least about 20%, at least about 30%, at least about 40%, at least about 50%, at least about 60%, at least about 70%, at least about 80%, at least about 90%, or at least about 95%, relative to untreated subjects or subjects receiving a different treatment. In addition, the methods described herein can result in at least stable disease, partial response, or complete response, as assessed by the WHO or RECIST criteria for tumor response (Natl. Cancer. Inst. 91:523-8, 1999; and Cancer 47:207-14, 1981). In some embodiments, a treatment effect is determined on the basis of an objective response, objective response rate, complete response, complete response rate, duration of response, duration of complete response, progression free survival, and overall survival.

The methods described herein can decrease tumor size or cancer burden, or otherwise ameliorate symptoms in a subject, or otherwise support partial or complete stable disease and/or partial or complete response as determined above.

Treatment with any of the pharmaceutical compositions described herein (e.g., comprising any of the antibodies or antigen-binding fragments described herein), optionally in combination with any of the other therapeutic agents or treatments described herein, can increase the median progression-free survival or overall survival time of patients with cancer, especially when relapsed or refractory, by at least 30%, at least 40%, at least 50%, at least 60%, at least 70%, at least 80%, at least 90%, or at least 95%, compared to the same treatment (e.g., chemotherapy) but without administration of any of the pharmaceutical compositions comprising any of the anti-BCMA antibodies or antigen-binding fragments described herein. In addition or alternatively, treatment (e.g., standard chemotherapy) including administration of any of the pharmaceutical compositions comprising any of the anti-BCMA antibodies or antigen-binding fragments described herein, can increase the complete response rate, partial response rate, or objective response rate (complete+partial) of patients with tumors by at least 30%, at least 40%, at least 50%, at least 60%, at least 70%, at least 80%, at least 90%, or at least 95%, compared to the same treatment (e.g., chemotherapy) but without administration of any of the pharmaceutical compositions comprising any of the anti-BCMA antibodies or antigen-binding fragments described herein.

Typically, in a clinical trial (e.g., a phase II, phase II/III, or phase III trial), the aforementioned increases in median progression-free survival and/or response rate of the patients treated with standard therapy plus any of the pharmaceutical compositions comprising any of the anti-BCMA antibodies or antigen-binding fragments described herein, relative to the control group of patients receiving standard therapy alone (or plus placebo), are statistically significant, for example at the p=0.05, 0.01, or 0.001 level. The complete and partial response rates are determined by objective criteria commonly used in clinical trials for cancer, e.g., as listed or accepted by the National Cancer Institute and/or Food and Drug Administration.

A patient is determined to have an objective response (OR) if, based on the 2016 IMWG uniform response criteria, they achieve a stringent complete response (sCR), complete response (CR), very good partial response (VGPR), or a partial response (PR). The objective response rate (ORR) is defined as the proportion of patients with an OR per investigator. Patients whose disease response cannot be evaluated per the 2016 IMWG uniform response criteria are scored as Not Evaluable for calculating the ORR. Patients who do not have post baseline response assessment, or the response is Not Evaluable per IMWG criteria are counted as non-responders in calculation of ORR. Objective response (OR) can be assessed by imaging, laboratory assessment, or physical examination; or SD and clinical improvement in disease-related symptoms per investigator.

In one embodiment of any of the methods described herein, the objective response rate (ORR) is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% after the administration of the antibodies or antigen-binding fragments described herein.

A patient is determined to have a complete response (CR) if, based on the 2016 IMWG uniform response criteria they achieve a sCR or CR. The CR rate is defined as the proportion of patients with a CR per investigator. Patients whose disease response cannot be evaluated per the IMWG uniform response criteria are scored as Not Evaluable for calculating the CR rate.

In one embodiment of any of the methods described herein, the complete response rate (CRR) is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% after the administration of the antibodies or antigen-binding fragments described herein.

Duration of OR is defined as the time from first documentation of OR (sCR, CR, VGPR, or PR) to the first documentation of disease progression or to death due to any cause, whichever comes first. Disease progression includes objective evidence of tumor progression (based on serum, urine, or bone marrow assessments) and/or clinical progression per investigator. Duration of response is only calculated for the subgroup of patients achieving a sCR, CR, VGPR, or PR.

In one embodiment of any of the methods described herein, the duration of objective response or the duration of complete response to the treatment is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years.

Progression-free survival (PFS) is defined as the time from the start of treatment to first documentation of disease progression or to death due to any cause, whichever comes first. Disease progression includes objective evidence of tumor progression (based on serum, urine or bone marrow assessments) and/or clinical progression per investigator. PFS is censored on the date of the last disease assessment documenting absence of progressive disease (PD) for patients who do not have disease progression and are still on study at the time of an analysis, or are removed from study prior to documentation of tumor progression. Patients who have started a new antitumor treatment prior to documentation of PD will be censored at the last disease assessment prior to start of new treatment. Patients lacking an evaluation of tumor response after their first dose have their event time censored at 1 day.

In one embodiment of any of the methods described herein, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years.

Overall survival (OS) is defined as the time from the start of any study treatment to the date of death due to any cause. Specifically,

OS=date of death−date of first dose of any study treatment+1.

In one embodiment of any of the methods described herein, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years.

### H. Exemplary Monotherapies and Combination Therapies

In certain embodiments, the subject receives a dose of a pharmaceutical composition comprising any of the antibodies or antigen-binding fragments that bind specifically to BCMA once every two weeks (q2wk) according to a standard dosing regimen. In some of the standard dosing treatments, each dose contains 800 mg of an anti-BCMA antibody or an antigen-binding fragment described herein. In other standard dosing treatments, each dose administered to the subject contains 1600 mg of an anti-BCMA antibody or an antigen-binding fragment described herein.

In some embodiments of any of the methods described herein, an intensive dosing of the anti-BCMA antibody or antigen-binding fragment thereof is performed. In certain embodiments, the intensive dosing comprises weekly induction dosing (q1wk) of any of the anti-BCMA antibodies or antigen-binding fragments described herein for 8 doses during the first 2 cycles of therapy (i.e. Cycle 1 and Cycle 2). Assuming the patient does not experience confirmed disease progression, the subject is administered any of the anti-BCMA antibodies or antigen-binding fragments described herein dosed q2wk during a maintenance phase during Cycle 3 and beyond. Dosing during the maintenance phase is typically at the standard dosing level, i.e., either 800 mg or 1600 mg of the antibody or antigen-binding fragment.

Thus, in some embodiments, intensive dosing of the anti-BCMA antibody or antigen-binding fragment thereof includes administering 800 or 1600 mg of an anti-BCMA antibody or an antigen-binding fragment described herein, on Day 1, Day 8, Day 15, and Day 22 of Cycle 1 and Cycle 2, and Day 1 and Day 15 of subsequent cycles.

In some embodiments, nirogacestat is combined with the standard or intensive anti-BCMA antibody or antigen-binding fragment thereof regimens as part of a combination therapy. In some embodiments of such combination treatments, nirogacestat is administered as a 100 mg dose and is administered twice a day. Thus, for example, some combination therapy embodiments involve a standard dosing combination therapy in which nirogacestat is administered in combination with a standard dosing regimen of the anti-BCMA antibodies or antigen-binding fragments described herein in which the antibody or antigen-binding fragment is administered q2wk. For example, in some standard dosing combination treatments, an anti-BCMA antibody or antigen-binding fragment as described herein is administered on Day 1 and Day 15 of each 28-day cycle (i.e., according to a standard dosing regimen) and nirogacestat is administered twice each day of each 28-day cycle. In some of these standard dosing combination embodiments, each dose of the antibody or antigen binding fragment is administered as an 800 mg dose and each dose of nirogacestat is administered as a 100 mg dose. In other embodiments of standard dosing combination treatments, each dose of the antibody or antigen binding fragment is administered as an 1600 mg dose and each dose of nirogacestat is administered as a 100 mg dose.

In some embodiments, dexamethasone is combined with the standard or intensive anti-BCMA antibody or antigen-binding fragment thereof regimens and nirogacestat as part of a combination therapy. In some embodiments of such combination treatments, dexamethasone is administered as a 40 mg dose and is administered once a week (i.e., q1wk). Thus, for example, some combination therapy embodiments involve a standard dosing combination therapy in which dexamethasone is administered in combination with a standard dosing regimen of the anti-BCMA antibodies or antigen-binding fragments described herein in which the antibody or antigen-binding fragment is administered q2wk and nirogacestat (e.g., 100 mg nirogacestat) is administered twice each day. For example, in some standard dosing combination treatments, an anti-BCMA antibody or antigen-binding fragment as described herein is administered on Day 1 and Day 15 of each 28-day cycle (i.e., according to a standard dosing regimen), nirogacestat is administered twice each day of each 28-day cycle as a 100 mg dose, and dexamethasone is administered on Day 1, Day 8, Day 15, and Day 22 of each 28-day cycle. In some of these standard dosing combination embodiments, each dose of the antibody or antigen binding fragment is administered as an 800 mg dose, nirogacestat is administered twice each day of each 28-day cycle as a 100 mg dose, and each dose of dexamethasone is administered as a 40 mg dose. In other embodiments of standard dosing combination treatments, each dose of the antibody or antigen binding fragment is administered as an 1600 mg dose, each dose of nirogacestat is administered as a 100 mg dose, and each dose of dexamethasone is administered as a 40 mg dose.

Other examples of combination therapy embodiments involve an intensive dosing combination therapy in which nirogacestat and dexamethasone are administered in combination with an intensive dose regimen of any of the anti-BCMA antibodies or antigen-binding fragments described herein in which the antibody or antigen-binding fragment is administered q1wk for 8 weeks, followed by q2wk dosing. For example, in some intensive dosing combinations, the anti-BCMA antibody or antigen-binding fragment as described herein is administered on Day 1, Day 8, Day 15, and Day 22 of Cycles 1 and 2, and Day 1 and Day 15 of subsequent cycles (i.e., according to an intensive dosing regimen), nirogacestat is administered twice daily on Day 1 to Day 28 of each 28-day cycle, and dexamethasone is administered on Day 1, Day 8, Day 15, and Day 22 of each 28-day cycle. In some of these intensive dosing combination embodiments, each dose of the antibody or antigen binding fragment is administered as an 800 mg dose, each dose of nirogacestat is administered as a 100 mg dose, and each dose of dexamethasone is administered as a 40 mg dose. In other of these embodiments, each dose of the antibody or antigen binding fragment is administered as an 1600 mg dose, each dose of nirogacestat is administered as a 100 mg dose, and each dose of dexamethasone is administered as a 40 mg dose.

In any one of the exemplary combination therapies, when the anti-BCMA antibody or antigen-binding fragment and dexamethasone are both administered on the same day, dexamethasone is administered 1 to 3 hours prior to SEA BCMA infusion.

In some embodiments, the anti-BCMA antibody or an antigen-binding fragment described herein is administered to the subject once every two weeks (e.g., Day 1 and Day 15 of each 28-day cycle), dexamethasone is administered once every week (e.g., on Day 1, Day 8, Day 15, and Day 22 of each 28-day cycle), and nirogacestat is administered to the subject twice daily on Day 1 to Day 28 of each 28-day cycle. In some embodiments, 1600 mg of an anti-BCMA antibody (e.g., SEA-BCMA) is administered to the subject once every two weeks (e.g., Day 1 and Day 15 of each 28-day cycle), 40 mg of dexamethasone is administered once every week (e.g., on Day 1, Day 8, Day 15, and Day 22 of each 28-day cycle) and 100 mg of nirogacestat is administered to the subject with twice daily on Day 1 to Day 28 of each 28-day cycle.

In some embodiments, the anti-BCMA antibody or an antigen-binding fragment described herein is administered to the subject once every week for about 8 weeks and then once every two weeks (e.g., on Day 1, Day 8, Day 15, and Day 22 of two 28-day cycles, and Day 1 and Day 15 of subsequent 28-day cycles), dexamethasone is administered once every week (e.g., on Day 1, Day 8, Day 15, and Day 22 of each 28-day cycle), and nirogacestat is administered to the subject twice daily on Day 1 to Day 28 of each 28-day cycle.

In some embodiments, 1600 mg of an anti-BCMA antibody (e.g., SEA-BCMA) is administered to the subject once every week for about 8 weeks and then once every two weeks (e.g., on Day 1, Day 8, Day 15, and Day 22 of two 28-day cycles, and Day 1 and Day 15 of subsequent 28-day cycles), 40 mg of dexamethasone is administered once every week (e.g., on Day 1, Day 8, Day 15, and Day 22 of each 28-day cycle), and 100 mg of nirogacestat is administered to the subject twice daily on Day 1 to Day 28 of each 28-day cycle.

In some embodiments, 800 mg of an anti-BCMA antibody (e.g., SEA-BCMA) is administered to the subject once every week for about 8 weeks and then once every two weeks (e.g., on Day 1, Day 8, Day 15, and Day 22 of two 28-day cycles, and Day 1 and Day 15 of subsequent 28-day cycles), 40 mg of dexamethasone is administered once every week (e.g., on Day 1, Day 8, Day 15, and Day 22 of each 28-day cycle), and 100 mg of nirogacestat is administered to the subject twice daily on Day 1 to Day 28 of each 28-day cycle.

In some embodiments, 800 mg of an anti-BCMA antibody (e.g., SEA-BCMA) is administered to the subject on Day 1, Day 8, Day 15, and Day 22 of two 28-day cycles, and 1600 mg of an anti-BCMA antibody (e.g., SEA-BCMA) is administered to the subject on Day 1 and Day 15 of subsequent 28-day cycle(s) of a maintenance phase, 40 mg of dexamethasone is administered to the subject on each of Day 1, Day 8, Day 15 and Day 22 of each 28-day cycle, and 100 mg of nirogacestat is administered to the subject twice daily on Day 1 to Day 28 of each 28-day cycle.

In some embodiments, 400 mg of an anti-BCMA antibody (e.g., SEA-BCMA) is administered to the subject on Day 1, Day 8, Day 15, and Day 22 of two 28-day cycles, and 800 mg of an anti-BCMA antibody (e.g., SEA-BCMA) is administered to the subject on Day 1 and Day 15 of subsequent 28-day cycle(s) of a maintenance phase, 40 mg of dexamethasone is administered to the subject on each of Day 1, Day 8, Day 15 and Day 22 of each 28-day cycle, and 100 mg of nirogacestat is administered to the subject twice daily on Day 1 to Day 28 of each 28-day cycle.

In some embodiments, 400 mg of an anti-BCMA antibody (e.g., SEA-BCMA) is administered to the subject on Day 1, Day 8, Day 15, and Day 22 of two 28-day cycles, and 400 mg of an anti-BCMA antibody (e.g., SEA-BCMA) is administered to the subject on Day 1 and Day 15 of subsequent 28-day cycle(s) of a maintenance phase, 40 mg of dexamethasone is administered to the subject on each of Day 1, Day 8, Day 15 and Day 22 of each 28-day cycle, and 100 mg of nirogacestat is administered to the subject twice daily on Day 1 to Day 28 of each 28-day cycle.

### I. Patient Selection for Different Dosing Regimens and Combination Therapies.

The diagnosis of multiple myeloma (MM) requiring systemic therapy can be based on International Myeloma Working Group (IMWG) 2014 criteria. The measurable disease can be defined by one or more of the following:
a) Serum monoclonal paraprotein (M-protein) level ≥0.5 g/dL; for IgA or IgD myeloma patients, serum IgA or serum IgD ≥0.5 g/dL is acceptable
b) Urine M-protein level ≥200 mg/24 hr
c) Serum immunoglobulin FLC ≥10 mg/dL and abnormal serum immunoglobulin kappa lambda FLC ratio

In some embodiments, an ECOG Performance Status score of 0 or 1 is needed before receiving the treatment as described herein.

In some embodiments, hematologic criteria must be met in the absence of growth factor or platelet transfusion support:
a) Estimated glomerular filtration rate (eGFR) ≥30 mL/min/1.73 m² per the Modification of Diet in Renal Disease (MDRD) equation.
b) Absolute neutrophil count ≥1000/μL
c) Platelet count ≥75,000/μL.

Patients can be selected for different dosing regimens or combination therapies. For example, standard dosing (e.g., q2wk, day 1 and day 15 of each 28-day cycle) can be administered to certain patients. In some embodiments, these patients must not have other therapeutic options known to provide clinical benefit in MM available. In some embodiments, patients' prior lines of therapy for patients must include at least a proteasome inhibitor (PI), an immunomodulatory drug (IMiD), and an anti-CD38 antibody in any order during the course of treatment. In some embodiments, the subject was previously administered at least one BCMA-directed myeloma therapy selected from the group consisting of: ADC, CAR-T cell therapy, and bispecific antibodies targeting human BCMA.

Intensive dosing (e.g., q1wk for the first two 28-day cycles, then q2wk in subsequent 28-day cycles) or the combination therapy with dexamethasone can be administered to certain patients. In some embodiments, these patients must not have other therapeutic options known to provide clinical benefit in MM available. In some embodiments, these patients must have received at least 3 prior lines of anti-myeloma therapy and must be refractory to at least 1 agent in each of the following classes: PI, IMiD, and an anti-CD38 antibody. In some embodiments, the subject was previously administered at least one BCMA-directed myeloma therapy selected from the group consisting of: ADC, CAR-T cell therapy, and bispecific antibodies. When the combination therapy with dexamethasone is administered to the patient, the antibody or antigen-binding fragment thereof as described herein can be administered under either the standard dosing schedule or the intensive dosing schedule.

In some embodiments, the combination therapy with dexamethasone and an IMiD can be administered to certain patients. In some embodiments, these patients must have received at least 2 prior lines of antimyeloma therapy, including at least 2 consecutive cycles of lenalidomide and a proteosome inhibitor (given separately or in combination), and must have documented IMWG disease progression on or within 60 days of completion of their last treatment. Patients with a history of autologous SCT (stem-cell transplantation) are eligible if the date of transplant was at least 12 weeks prior to initiation of SEA-BCMA treatment.

### Assays

The physical conditions of the subject treated by the methods described herein can be measured by any suitable assays known in the art. Non-limiting assays include immunohistochemical assays, radio imaging assays, in-vivo imaging, positron emission tomography (PET), single photon emission computer tomography (SPECT), magnetic resonance imaging (MRI), Ultra Sound, Optical Imaging, Computer Tomography, radioimmunoassay (RIA), ELISA (enzyme-linked immunosorbent assay), slot blot, competitive binding assays, fluorimetric imaging assays, Western blot, FACS, and the like.

In some embodiments, a biological sample is collected from the subject for an assay. The biological samples include, but are not limited to blood, serum, urine, plasma, the external secretions of the respiratory, intestinal, and genitourinary tracts, cerebrospinal fluid, peritoneal fluid, pleural fluid, cyst fluid, broncho alveolar lavage, lavage of any other part of the body or system in the body, and samples of any organ including isolated cells or tissues, where the cell or tissue can be obtained from an organ selected from, but not limited to lung, colon, kidney, pancreas, ovary, prostate, liver, skin, bone marrow, lymph node, breast, and/or blood tissue; stool or a tissue sample, or any combination thereof. Prior to performance of the assay, the sample can optionally be diluted with a suitable diluent. In some embodiments, cells obtained from the sample are cultured in vitro prior to performing the assay.

In some embodiments, the steady-state concentration of the anti-BCMA antibody in the serum of the subject can be measured.

One exemplary in vitro cell binding capacity assay to estimate the free anti-BCMA antibody in patients serum Involves pelleting a suspension of cultured MM1R cells and then re-suspending the pellet in serum from peripheral blood of subjects collected at different time points in treatment. After incubation at room temperature for 0.5 hour, the cells are washed and stained with a saturating amount of one of the anti-BCMA antibodies described herein conjugated to a fluorescent dye. After incubation at 4° C. in the dark for 0.5 hr, the cells are washed and fixed. Stained cells are analyzed on an Invitrogen Attune NxT flow cytometer. FlowJo V10 software is used to gate on viable cells and record the median fluorescent intensity (MFI). GraphPad Prism 8 is used for analysis.

One exemplary method of determining BCMA expression and binding by its ligands and an anti-BCMA antibody as described herein involves collecting bone marrow aspirates from a subject at baseline and after or during treatment, and then testing the samples by flow cytometry within one day of collection. MM cell detection can be performed using extracellular biomarker staining, for example, CD138, CD38, CD45, CD56, and CD28 staining and intracellular kappa and lambda light chains staining. Profiling of BCMA expression can be performed using, for example, two anti-BCMA antibodies: BCMA available for binding to anti-BCMA antibodies is detected using labeled anti-BCMA antibodies that bind BCMA in a competitive manner with a reference anti-BCMA antibody (e.g., one of the antibodies or antigen-binding fragments described herein such as the SEA-BCMA antibody described in the examples) and BCMA ligands (APRIL, etc.), while total extracellular BCMA is detected using a differently labeled anti-BCMA antibody that binds BCMA without competing with the reference antibody and BCMA ligands. Detection of APRIL, bound to BCMA on the MM cell surface, can also be performed. Each sample is split into 3 aliquots: one aliquot stained using only the MM gating antigens but no anti-BCMA or anti-APRIL antibodies (gating control), one aliquot stained with MM gating antigens and both labeled anti-BCMA antibodies, and one incubated for, for example, 2 hours at 37° C. with spiked BCMA (e.g., 100 μg/mL of spiked BCMA) before staining with MM gating antigens, APRIL, and the labeled anti-BCMA antibody detecting total extracellular BCMA. After staining, the cells are washed and fixed in 2% paraformaldyde, and the cells are analyzed on a flow cytometer.

### Kits

Also provided herein are kits that include: (a) one or more doses (e.g., 2, 3, 4, 5, 6, 7, 8, 9, 10, 11, 12, 13, 14, 15, 16, 17, 18, 19, 20, 21, 22, 23, 24, 25, or 26 doses) of a pharmaceutical composition (e.g., any of the pharmaceutical compositions described herein) comprising any of the antibodies or antigen-binding fragments thereof described herein that specifically binds to BCMA, and (b) instructions or directions for performing any one of the methods described herein. In some embodiments, the antibody or antigen-binding fragment thereof comprises a heavy chain variable region comprising a CDR1 comprising SEQ ID NO: 1, a CDR2 comprising SEQ ID NO: 2, and a CDR3 comprising SEQ ID NO: 3, and a light chain variable domain comprising a CDR1 comprising SEQ ID NO: 5, a CDR2 comprising SEQ ID NO: 6, and a CDR3 comprising SEQ ID NO: 7. In some embodiments of any of the kits described herein, the kit further includes one or more doses (e.g., 2, 3, 4, 5, 6, 7, 8, 9, 10, 11, 12, 13, 14, 15, 16, 17, 18, 19, 20, 21, 22, 23, 24, 25, or 26 doses) of a pharmaceutical composition comprising nirogacestat. In some embodiments of any of the kits described herein, the kit further includes one or more doses (e.g., 2, 3, 4, 5, 6, 7, 8, 9, 10, 11, 12, 13, 14, 15, 16, 17, 18, 19, 20, 21, 22, 23, 24, 25, or 26 doses) of pharmaceutical composition comprising dexamethaseone.

In some embodiments, the one or more doses of the pharmaceutical composition comprising any of the antibodies or antigen-binding fragments described herein that bind specifically to BCMA and/or dexamethasone can be provided in an injection device (e.g., a preloaded injection device). In some embodiments, the one or more doses of the pharmaceutical composition comprising any of the antibodies or antigen-binding fragments described herein that bind specifically to BCMA and/or dexamethasone can be provided as a lyophilized solid composition that can be reconstituted using a pharmaceutically acceptable buffer or solution (e.g., saline or phosphate buffered saline). In some embodiments, the one or more doses of the pharmaceutical composition comprising any of the antibodies or antigen-binding fragments described herein that bind specifically to BCMA and/or dexamethasone can be provided as a liquid composition (e.g., a liquid composition that can be administered to the subject via intravenous administration).

In some embodiments, the one or more doses of the pharmaceutical composition comprising nirogacestat ((S)-2-(((S)-6,8-difluoro-1,2,3,4-tetrahydronaphthalen-2-yl)amino)-N-(1-(2-methyl-1-(neopentylamino)propan-2-yl)-1H-imidazol-4-yl)pentanamide), (PF-03084014), can be formulated for oral administration (e.g., any of the pharmaceutically acceptable salt forms of nirogacestat described herein or known in the art, e.g., nirogacestat hydrobromide or nirogacestat dihydrobromide). In some embodiments, the one or more doses of the pharmaceutical composition comprising nirogacestat or a pharmaceutically acceptable salt thereof is formulated as a tablet, capsule, or aqueous suspension. Non-limiting examples of carriers that can be present in a pharmaceutical composition comprising nirogacestat include microcrystalline cellulose, sodium citrate, calcium carbonate, dicalcium phosphate, and glycine. Non-limiting examples of disintegrants that can be present in a pharmaceutical composition comprising nirogacestat include starch (preferably corn, potato, or tapioca starch), methylcellulose, alginic acid, and certain complex silicates. Non-limiting examples of granulation binders that can be present in a pharmaceutical composition comprising nirogacestat include polyvinylpyrrolidone, sucrose, gelatin, and acacia. Lubricating agents such as magnesium stearate, sodium lauryl sulfate and talc are often useful for tableting purposes. Solid compositions of a similar type may also be employed as fillers in gelatin capsules. Preferred materials in this connection include lactose or milk sugar as well as high molecular weight polyethylene glycols. When aqueous suspensions and/or elixers are desired for oral administration, the active ingredient may be combined with various sweetening or flavoring agents, coloring matter or dyes, and, if so desired, emulsifying and/or suspending agents as well, together with such diluents as water, ethanol, glycerin, and various like combinations thereof.

### EXAMPLES

### Example 1. Clinical Study of SEA-BCMA in Treatment of Multiple Myeloma

SEA-BCMA is a non-fucosylated monoclonal anti-BCMA antibody having the heavy chain amino acid sequence of SEQ ID NO: 13, and the light chain amino acid sequence of SEQ ID NO: 15.

Heavy Chain of SEA-BCMA
(SEQ ID NO: 13)
QVQLVQSGAEVKKPGASVKLSCKASGYTFTDYYIHWVRQAPGQGLEWIGYINPNSGYT
NYAQKFQGRATMTADKSINTAYVELSRLRSDDTAVYFCTRYMWERVTGFFDFWGQGT
MVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPPCP
APELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK
Light Chain of SEA-BCMA
(SEQ ID NO: 15)
DIQMTQSPSSVSASVGDRVTITCLASEDISDDLAWYQQKPGKAPKVLVYTTSSLQ
SGVPSRFSGSGSGTDFTLTISSLQPEDFATYFCQQTYKFPPTFGGGTKVEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC

SEA-BCMA comprises a heavy chain variable region comprising a CDR1 comprising DYYIH (SEQ ID NO: 1), a CDR2 comprising YINPNSGYTNYAQKFQG (SEQ ID NO: 2), and a CDR3 comprising YMWERVTGFFDF (SEQ ID NO: 3), and a light chain variable region comprising a CDR1 comprising LASEDISDDLA (SEQ ID NO: 5), a CDR2 comprising TTSSLQS (SEQ ID NO: 6), and a CDR3 comprising QQTYKFPPT (SEQ ID NO: 7). SEA-BCMA comprises a heavy chain variable region comprising SEQ ID NO: 4, and a light chain variable region comprising SEQ ID NO: 8.

A clinical study to evaluate SEA-BCMA in a patient population whose disease has relapsed or is refractory to standard therapies, and for whom there remains no treatment options available, is ongoing and the initial data indicate that methods of treating multiple myeloma described herein provide a clinical benefit.

The immunospecificity and antitumor activity of SEA-BCMA have been demonstrated both in vitro and in vivo in BCMA-expressing MM models.

This study evaluated the safety and antitumor activity of SEA-BCMA in patients with RRMM. Specific objectives and corresponding endpoints for the study are summarized below (Table 1).

TABLE 1
Objectives and corresponding endpoints

| Primary Objectives | Corresponding Primary Endpoint |
|---|---|
| Evaluate the safety and tolerability of | Type, incidence, severity, seriousness, and |
| SEA-BCMA monotherapy in patients with | relatedness of adverse events (AEs) |
| relapsed or refractory multiple myeloma | Type, incidence, and severity of laboratory |
| (RRMM) | abnormalities |
| Identify the maximum tolerated dose (MTD) | Incidence of dose-limiting toxicities (DLTs) |
| and/or optimal dose and schedule of SEA- | |
| BCMA monotherapy in patients with RRMM | |
| Evaluate the safety and tolerability of SEA- | Type, incidence, severity, seriousness, and |
| BCMA in combination with dexamethasone in | relatedness of adverse events (AEs) |
| patients with RRMM | Type, incidence, and severity of laboratory |
| | abnormalities |
| Secondary Objectives | Corresponding Secondary Endpoints |
| Identify a recommended single-agent dose and | Incidence of DLTs, cumulative safety and |
| schedule of SEA-BCMA | activity by dose level |
| Assess the pharmacokinetics (PK) of SEA- | Maximum serum concentration and area |
| BCMA | under the serum concentration-time curve |
| Assess the immunogenicity of SEA-BCMA | Incidence of SEA-BCMA antitherapeutic |
| | antibodies (ATA) |
| Assess the antitumor activity of SEA-BCMA | Best response per the International Myeloma |
| | Working Group (IMWG) uniform response |
| | criteria (Kumar 2016) |
| | Objective response rate (ORR) |
| | Duration of objective response (OR) and |
| | complete response (CR) |
| | Progression-free survival (PFS) |
| | Overall survival (OS) |
| Exploratory Objectives | Corresponding Exploratory Endpoints |
| Assess incidence and level of BCMA expression | Characterization of BCMA expression on |
| in RRMM and relationship to clinical response to | malignant plasma cells |
| SEA-BCMA | |
| Assess the pharmacodynamic effects and | Exploratory biomarkers of SEA-BCMA- |
| biomarkers of response, toxicity, and resistance to | mediated pharmacodynamic effects |
| SEA-BCMA | |
| Assess minimal residual disease (MRD) in | Rate of MRD clearance |
| patients with very good partial response (VGPR) | Descriptive outcomes of qualitative interviews |
| or better | Maximum serum concentration and area |
| Assess impact of SEA-BCMA in combination | under the serum concentration-time curve |
| with SOC therapies and SEA-BCMA in | |
| combination with dexamethasone on health related | |
| quality of life (HRQOL) from the patient's | |
| perspective | |
| Assess impact of SEA-BCMA in combination | |
| with SOC therapies and SEA-BCMA in | |
| combination with dexamethasone and nirogacestat | |
| on HRQOL from the patient's perspective | |
| Assess the PK of nirogacestat in combination with | |
| SEA-BCMA and dexamethasone | |

### Summary of Study Design

### Monotherapy Dose-Escalation Cohort

The monotherapy dose-escalation portion of the trial was conducted in approximately 25 patients.

Enrollment in this study occurred on a cohort-by-cohort basis. Multiple cohorts were treated at each dose level, with a maximum of 4 patients treated per cohort. Decisions on dose escalation and subsequent cohort size were made in consultation with the safety monitoring committee (SMC) after completion of each cohort. Patients in the current cohort were observed for the full duration of the DLT period before the next cohort of patients was enrolled. In addition, as a precaution, for the first 2 patients in the study there was a 72-hour observation period before the next patient can be dosed. At dose levels above Dose Level 1, a 24-hour observation period was required after the first patient received their first dose of SEA-BCMA, prior to dosing subsequent patients at that dose level. At least 2 DLT-evaluable (DE) patients were treated per dose level until the first DLT was observed, then a minimum of 3 DE patients per dose level was required before escalation to all higher doses. Patients who were considered not evaluable for DLT during Cycle 1 were replaced. A minimum of 6 DE patients were observed at the estimated MTD before the MTD or optimal dose was determined. The MTD or optimal dose was estimated based on data from all patients across all evaluated doses.

De-escalation to a lower dose level could be performed at any time in consultation with the SMC. Intrapatient dose escalation to a dose level shown to be safe could be permitted in the event that a patient tolerates SEA-BCMA and achieves stable disease (SD) or better.

Patients continued on treatment until progressive disease or unacceptable toxicity, whichever occurred first.

SEA-BCMA was initially administered once every 2 weeks (q2wk) in 4-week cycles at the planned doses shown in Table 2; a dosing interval of every 4 weeks (q4wk) was explored.

TABLE 2
Dose escalation schema

| Dose Levelª | Dose (mg) |
|---|---|
| 1 | 100 |
| 2 | 200 |
| 3 | 400 |
| 4 | 800 |
| 5 | 1,600 |

^{a}The Safety Monitoring Committee may recommend investigation of intermediate dose levels based on emerging clinical data.

### Monotherapy Expansion Cohort

To further characterize the safety and antitumor activity of SEA-BCMA, an expansion cohort of up to approximately 40 patients were enrolled. The dose and schedule for the expansion cohort were determined in consultation with the SMC based on the cumulative safety and activity demonstrated during dose escalation, which was completed without exceeding MTD at the doses tested.

### Monotherapy Intensive Dosing

The intensive dosing evaluates the safety and tolerability of SEA-BCMA dosed once a week (q1wk) during an induction phase (for 8 doses during the first 2 cycles of therapy); following the completion of the 8 week induction phase, patients who have not yet experienced confirmed disease progression proceeded to receive SEA-BCMA dosed q2wk during a maintenance phase (Cycle 3 and beyond, dosing at the recommended standard-schedule monotherapy expansion dose).

The intensive dosing includes a safety run-in at the recommended SEA BCMA monotherapy expansion dose (1600 mg), administered on the intensive dosing schedule (Day 1, Day 8, Day 15, and Day 22 of Cycles 1 and 2, and Day 1 and Day 15 of subsequent cycles). DLTs are being evaluated in the first 6 patients.

Patients who are deemed not evaluable for dose-limiting toxicity (DLT) during dose finding will be replaced for the determination of the dose of SEA-BCMA in combination with dexamethasone.

TABLE 3
Dose levels for monotherapy intensive dosing

| | Weekly Induction | Biweekly Maintenance |
|---|---|---|
| | Dose, Cycles 1-2 | Dose, Cycles 3 |
| Dose Level | (mg) | and beyond (mg) |
| 1 | 1600 | 1600 |
| −1 (if Dose Level 1 | 800 | 1600 |
| is not tolerated) | | |

### Dexamethasone Combination Therapy Cohorts

To characterize the safety and tolerability of SEA-BCMA in combination with dexamethasone, approximately 20 patients will be initially enrolled in each optional combination therapy cohort.

Enrollment into combination therapy cohorts will be initiated upon identification of tolerable SEA-BCMA monotherapy doses and schedules.

In Optional Cohort 1, SEA-BCMA will be administered on Day 1 and Day 15 of each 28-day cycle (standard dosing; 1600 mg). Dexamethasone will be administered on Day 1, Day 8, Day 15, and Day 22 of each 28-day cycle.

In Optional Cohort 2, SEA-BCMA will be administered at 800 mg (one dose below the recommended monotherapy expansion dose) on Day 1, Day 8, Day 15, and Day 22 of Cycles 1 and 2 (intensive dosing), and at 1600 mg Day 1 and Day 15 of subsequent cycles (the recommended monotherapy expansion dose). Dexamethasone will be administered on Day 1, Day 8, Day 15, and Day 22 of each 28-day cycle.

This expansion cohort included an initial 3 subject safety run-in at 800 mg SEA-BCMA intensive dosing (dose level-1) and, if deemed tolerable, was followed by a 6-subject run in at 1600 mg SEA-BCMA intensive dosing. If 2 or more dose-limiting toxicity (DLTs) occur among the first 3 patients, then Cohort 2 will be discontinued. If 1 DLT occurs among the first 3 patients, the cohort will be expanded to 6 patients, and only escalated if there are fewer than 2 DLTs among the 6 patients. If 0 DLTs occur among the first 3 patients, the dose will be escalated to 1600 mg q1wk for 2 cycles and then 1600 mg q2wk for subsequent cycles, and the 6-subject safety run-in rules outlined below will be applied (see “Dose Limiting Toxicity” section).

Dexamethasone will administered at a dose of 40 mg on days 1, 8, 15, and 22 of each 28-day cycle as an intravenous (IV) infusion or PO. On days when SEA-BCMA is to be administered, dexamethasone will be administered 1 to 3 hours prior to SEA-BCMA infusion.

### Nirogacestat and Dexamethasone Combination Therapy Cohort

The nirogacestat and dexamethasone combination therapy portion of the trial will be conducted in approximately 40 patients. This cohort will SEA-BCMA with 100 mg orally (PO) nirogacestat twice a day and dexamethasone (IV) at standard 40 mg weekly dosing with SEA-BCMA administered q1wk for 8 weeks intensive dosing, followed by q2wk dosing. This expansion cohort will begin with a 6-subject run in at the dose of SEA-BCMA recommended for expansion from Cohort 2 of the dexamethasone combination therapy cohort.

Nirogacestat will be administered at a dose of 100 mg two times a day on each day of the 28-day cycle taken PO. The Cₘᵢₙ for nirogacestat at steady-state following a 100 mg BID dose is 232 ng/mL or 471 nM. Based on in vitro experiments with a panel of BMCA-expressing multiple myeloma and lymphoma cell lines, a dose of 100 mg BID would maintain nirogacestat concentration at or above the levels required to maximally inhibit the cleavage of BCMA, leading to reduced sBCMA and increased mbBCMA. With daily dosing, more consistent BCMA modulation may be possible that has been demonstrated with other GSIs. Daily dosing of nirogacestat provides adequate drug exposure for continued inhibition of gamma secretase, yielding sustained and rapid increases in mbBCMA and reduced levels of sBCMA over time. At the proposed dose level of 100 mg BID, nirogacestat is expected to have a safety profile at least as well-tolerated as the 150 mg BID dose used in solid tumor studies, some of which have had durations of treatment and follow-up longer than 5 years.

Dexamethasone will be administered at a dose of 40 mg on days 1, 8, 15, and 22 of each 28-day cycle as an IV infusion. On days when SEA-BCMA is to be administered, dexamethasone will be administered 1 to 3 hours prior to SEA-BCMA infusion.

### Combination Therapy Cohorts Safety Run-In

The combination therapy cohorts will include a safety run-in at the recommended SEA BCMA monotherapy dose and schedule. DLTs will be evaluated in the first 6 patients enrolled in each combination therapy cohort. If 0 or 1 of the first 6 subjects experience DLTs, the expansion cohort will proceed to enroll up to 20 patients with the recommendation of the SMC. If ≥2 DLTs occur in the first 6 subjects, MTD for the combination will be considered exceeded and the dose of SEA-BCMA will be de-escalated to the next lower dose level. If 0 or 1 of the first 6 subjects at the lower dose level experience a DLT, the expansion cohort will proceed to enroll up to 20 subjects at this dose level with the recommendation of the SMC. If ≥2 DLTs occur in the first 6 subjects at the lower dose level, MTD for the combination will be considered exceeded and the SMC will determine whether a further de-escalation will be tested, or if the combination cohort will be discontinued.

Patients who are deemed not evaluable for DLT during dose finding will be replaced for the determination of the dose of SEA-BCMA in combination with dexamethasone.

### Dose-Limiting Toxicity (DLT)

The DLT-evaluation period was the first cycle of treatment. DLTs were graded according to the National Cancer Institute Common Terminology Criteria for Adverse Events (NCI-CTCAE), version 4.03, and defined as any of the following events during the DLT-evaluation period:
A delay of treatment by more than 7 days due to toxicity
Any adverse event (AE) ≥Grade 3, unless deemed by the SMC to be clearly unrelated to SEA-BCMA, except for the following AEs, which must meet these specified criteria to be considered a DLT:
   Grade 4 neutropenia lasting more than 5 days
   Thrombocytopenia ≥Grade 4, or Grade 3 thrombocytopenia with clinically significant bleeding
   Anemia ≥Grade 4 unrelated to underlying disease
   Any Grade ≥3 tumor lysis syndrome, including associated laboratory evaluations, that is not successfully managed clinically and that does not resolve within 7 days without end organ damage
   Any ≥Grade 4 infusion-related reactions (IRRs) or Grade 3 IRRs that do not resolve to ≤Grade 2 within 24 hours with infusion interruption, infusion rate reduction, and/or standard supportive measures. In the event of a Grade 3 IRR in ≥20% of patients (i.e., 2 or more in the first 10 patients), all subsequent patients will require premedication and/or modification of infusion approach per the recommendation of the SMC. For patients receiving premedication, any ≥Grade 3 IRR will be considered a DLT.
   Any Grade ≥3 asymptomatic laboratory abnormality that does not resolve, with or without intervention, to ≤Grade 1 or the baseline grade within 72 hours
   Any treatment-related death

### Stopping Criteria

The study was halted if any of the following occurred:
Rate of on-study toxic deaths unrelated to underlying disease occurring within 30 days of dose exceeded 10% (initially, 2 or more of the first 20 patients)
Rate of Grade 4 non-hematologic toxicity unrelated to underlying disease exceeded 25% (initially, 5 or more of the first 20 patients)
Rate of ≥Grade 4 allergic reactions that cannot be controlled with standard treatments exceeded 15% (initially, 3 or more of the first 20 patients)
Stopping criteria were continuously monitored throughout the study by the sponsor.

### Discussion and Rationale for Study Design

Initial clinical development of SEA-BCMA involved its evaluation in patients with RRMM that have no other therapeutic options known to provide clinical benefit available, and were candidates for SEA-BCMA treatment in the opinion of the treating physician. Prior therapies must include at least a proteasome inhibitor (PI), an immunomodulatory drug (IMiD), and an anti-CD38 antibody. Frontline and first relapse standard of care (SOC) treatments were expected to have failed in these patients prior to enrollment. Because BCMA is a broadly expressed tumor antigen in patients with MM, initial selection of patients based on BCMA expression was not required, although the relationship between target expression and outcome were explored in this phase 1 study.

The first portion of the study consisted of dose escalation in order to estimate the MTD and/or optimal dose of SEA-BCMA. Once dose escalation was complete and safety of the drug was demonstrated, an expansion cohort of approximately 40 patients were enrolled to further evaluate the safety and antitumor activity of SEA-BCMA at the standard q2wk dosing schedule. The expansion cohort allowed for the collection of additional information about the safety, tolerability, and activity of SEA-BCMA. This information was the basis for determining the recommended single-agent dose and schedule for SEA-BCMA. Because maintenance therapy had been shown to prolong remissions in patients with MM, patients were permitted to continue on treatment until progressive disease (PD) or unacceptable toxicity, which ever occurred first. In addition, intrapatient dose escalation to a dose level shown to be safe was permitted in the event that a patient tolerated SEA-BCMA and achieved a response of SD or better.

### Study Population

All patients met all of the enrollment criteria to be eligible for this study and prior to study drug administration (within 1 day of dosing) on Cycle 1 Day 1.

To be eligible for retreatment, all patients met inclusion and exclusion criteria outlined in the below sections.

### Inclusion Criteria

1. Diagnosis of multiple myeloma (MM) requiring systemic therapy as defined by International Myeloma Working Group (IMWG) 2014 criteria (Kumar 2016).
2. Subjects must have MM that is relapsed or refractory and must not have other therapeutic options known to provide clinical benefit in MM available, and be a candidate for SEA-BCMA treatment in the opinion of the treating physician.
(a) Subjects that are enrolled in the dose escalation cohorts and does expansion cohorts must not have other therapeutic options known to provide clinical benefit in MM available. Subjects' prior lines of therapy for patients enrolled in the dose escalation study must include at least a proteasome inhibitor (PI), an immunomodulatory drug (IMiD), and an anti-CD38 antibody in any order during the course of treatment. Subjects who could not tolerate a PI, IMiD, or anti-CD38 antibody are allowed.
(b) Patients enrolled in the monotherapy intensive dosing or dexamethasone combination therapy must not have other therapeutic options known to provide clinical benefit in MM available. Patients must not have other therapeutic options known to provide clinical benefit in MM available. Patients must have received at least 3 prior lines of antimyeloma therapy and must be refractory to at least 1 agent in each of the following classes: PI, IMiD, and an anti-CD38 antibody.
(c) Patients enrolled in the combination therapy cohort with dexamethasone and nirogacestat may have received prior BCMA-directed myeloma therapy, excluding prior treatment with SEA-BCMA, (e.g., ADC, CAR-T therapy, or bispecific antibody therapy targeting BCMA) provided that at least 6 months will have elapsed between the last dose of prior BCMA-targeting therapy and Cycle 1 Day1 of this study, and that the patient has recovered from any clinically significant toxicity of the prior BCMA-targeting therapy.

Measurable disease, as defined by one or more of the following:
a. Serum monoclonal paraprotein (M-protein) level ≥0.5 g/dL; for IgA or IgD myeloma subjects, serum IgA or serum IgD ≥0.5 g/dL is acceptable.
b. Urine M-protein level ≥200 mg/24 hr
c. Serum immunoglobulin free light chain ≥10 mg/dL and abnormal serum immunoglobulin kappa lambda free light chain ratio
Age 18 years or older.
An Eastern Cooperative Oncology Group (ECOG) Performance Status score of 0 or 1 (e.g., conversion of performance status using Karnofsky and Lansky scales, if applicable).
Life-expectancy of >3 months in the opinion of the investigator

The following baseline laboratory data (hematologic criteria must be met in the absence of growth factor or platelet transfusion support):
a. Estimated glomerular filtration rate (eGFR) ≥30 mL/min/1.73 m² per the Modified Diet in Renal Disease (MDRD) equation
b. Absolute neutrophil count (ANC) ≥1000/μL
c. Platelet count ≥75,000/μL

Subjects of childbearing potential, under the following conditions:
a. Must have a negative serum or urine pregnancy test (minimum sensitivity 25 mIU/mL or equivalent units of beta human chorionic gonadotropin [β-hCG]) result within 10 to 14 days prior to the first dose of SEA-BCMA and one 24 hours prior to the start of the first dose of SEA-BCMA. Subjects with false positive results and documented verification that the subject is not pregnant are eligible for participation.
b. Must agree not to try to become pregnant during the study and for at least 6 months after the final dose of any study drug administration.
c. Must agree not to breastfeed or donate ova, starting at time of informed consent and continuing through 6 months after the final dose of any study drug administration.
d. If sexually active in a way that could lead to pregnancy, must consistently use 2 highly effective methods of birth control starting at time of informed consent and continuing throughout the study and for at least 6 months after the final dose of any study drug administration.

Patients who can father children, under the following conditions:
a. Must agree not to donate sperm starting at time of informed consent and continuing throughout the study period and for at least 6 months after the final dose of study drug administration.
b. If sexually active with a person of childbearing potential in a way that could lead to pregnancy, must consistently use 2 highly effective methods of birth control starting at time of informed consent and continuing throughout the study and for at least 6 months after the final dose of any study drug administration.
c. If sexually active with a person who is pregnant or breastfeeding, must consistently use one of 2 contraception options starting at time of informed consent and continuing throughout the study and for at least 6 months after the final dose of any study drug administration.

Furthermore, the subject must provide written informed consent.

### Exclusion Criteria

History of another malignancy within 3 years before the first dose of SEA-BCMA, or any evidence of residual disease from a previously diagnosed malignancy. Exceptions are malignancies with a negligible risk of metastasis or death (e.g., 5-year overall survival ≥90%), such as adequately treated carcinoma in situ of the cervix, non-melanoma skin carcinoma, localized prostate cancer, ductal carcinoma in situ, or Stage I uterine cancer.

Active cerebral/meningeal disease related to the underlying malignancy. Subjects with a history of cerebral/meningeal disease related to the underlying malignancy are allowed if prior central nervous system disease has been treated.

Any uncontrolled Grade 3 or higher (per the NCICTCAE, Version 4.03) viral, bacterial, or fungal infection within 2 weeks prior to the first dose of SEA-BCMA. Routine antimicrobial prophylaxis is permitted.

Positive for hepatitis B by surface antigen expression. Active hepatitis C infection (positive by polymerase chain reaction or on antiviral therapy for hepatitis C within the last 6 months).

Subjects who have been treated for hepatitis C infection are permitted if they have documented sustained virologic response of 12 weeks.

Known to be positive for human immunodeficiency virus (HIV).

Subjects with previous allogeneic stem cell transplant (SCT).

Documented history of a cerebral vascular event (stroke or transient ischemic attack), unstable angina, myocardial infarction, or cardiac symptoms consistent with congestive heart failure, Class III-IV, New York Heart Association (see Appendix F) within 6 months prior to their first dose of SEA-BCMA.

Current therapy with other systemic anti-neoplastic or investigational agents.

Chemotherapy, radiotherapy, biologics, investigational agents, and/or other antitumor treatment with immunotherapy that is not completed 4 weeks prior to first dose of SEA-BCMA, or 2 weeks if progressing and recovered from clinically significant toxicity associated with the treatment. CAR T-cell therapy that is not completed 8 weeks prior to first dose of SEA-BCMA. Palliative radiotherapy to a single site of disease is allowed with the approval of the medical monitor.

Systemic treatment with either corticosteroids (>10 mg daily prednisone equivalent) or other immunosuppressive medications within 14 days of enrollment. Inhaled or topical steroids and adrenal replacement steroid doses ≤10 mg daily prednisone equivalent are permitted.

Subjects who are breastfeeding, pregnant, or planning to become pregnant from time of informed consent until 6 months after final dose of study drug administration.

Known hypersensitivity to any excipient contained in the drug formulation of SEA-BCMA or nirogacestat.

Subjects with plasma cell leukemia (>2.0×10⁹/L circulating plasma cells by standard differential), Waldenstrom's macroglobulinemia, POEMS syndrome (polyneuropathy, organomegaly, endocrinopathy, monoclonal protein, and skin changes), or clinically significant amyloidosis.

Moderate or severe hepatic impairment, as indicated by any of the following:
a. Serum total bilirubin >1.5×upper limit of normal (ULN). For subjects with Gilbert's disease, total bilirubin >3×ULN.
b. Alanine aminotransferase (ALT) or aspartate aminotransferase (AST) >3×ULN

Significant comorbid condition or disease which in the judgment of the investigator would place the subject at undue risk or interfere with the proper assessment of safety and toxicity of the SEA-BCMA.

For combination therapy only: known intolerance to corticosteroids.

For combination therapy only: any uncontrolled psychoses.

For combination therapy only: Gastrointestinal disease that may predispose for drug intolerability or poor drug absorption (e.g., inability to take oral medication, prior surgical procedures affecting absorption (e.g., gastric bypass), malabsorption syndrome, and active peptic ulcer disease).

For combination therapy with dexamethasone and nirogacestat only: Prior treatment with nirogacestat or known intolerance to gamma secretase inhibitors.

For combination therapy with dexamethasone and nirogacestat only: Subject has an abnormal QT interval at screening (>470 ms by Fridericia formula).

For combination therapy with dexamethasone and nirogacestat only: Subject has a history of congenital or acquired prolonged QTc syndrome.

For combination therapy with dexamethasone and nirogacestat only: Concomitant medications that are known to prolong the QT/QTcF interval including Class Ia and Class III antiarrhythmics at the time of informed consent. Non-arrhythmic medications which may prolong the QT/QTcF interval are allowed provided the participant does not have additional risk factors for Torsades de Pointes (TdP).

For combination therapy with dexamethasone and nirogacestat only: Subjects who are receiving current ongoing therapy with strong inducers or moderate to strong inhibitors of CYP3A4 or strong inhibitors or inducers of P glycoprotein (P-gp). Strong inducers or moderate to strong inhibitors of CYP3A4 and/or strong inducers or inhibitors of P-gp are not allowed from 14 days prior to enrollment to the end of protocol therapy. However, CYP3A4 inducing anti-epileptic drugs on a stable dose are allowed.

### Discontinuation of Study Treatment

A patient's study treatment may be discontinued for any of the following reasons:
Progressive disease (PD)
AE
Pregnancy
Investigator decision
Patient decision, non-AE
Study termination by sponsor
Other, non-AE

In monotherapy, patients who discontinued SEA-BCMA were considered discontinued from study treatment. Patients who discontinue from study treatment will remain on study for follow-up until withdrawal of consent, death, or study closure, whichever occurs first.

In combination therapy, patients who discontinued SEA BCMA and dexamethasone will be considered discontinued from study treatment. Patients receiving dexamethasone who discontinued corticosteroid therapy may continue to receive SEA-BCMA as monotherapy with medical monitor approval. Patients who discontinued SEA-BCMA will be considered discontinued from study treatment.

In combination therapy with dexamethasone and nirogacestat, patient who discontinue SEA-BCMA, dexamethasone, and nirogacestat will be considered discontinued from study treatment. Patients receiving dexamethasone who discontinue corticosteroid therapy may continue to receive SEA-BCMA and nirogacestat with medical monitor approval. Patients who discontinue nirogacestat may continue to receive SEA-BCMA and dexamethasone with medical monitor approval. Patients who discontinue SEA-BCMA will be considered discontinued from study treatment.

Patient Withdrawal from Study

Any patient may be discontinued from the study for any of the following reasons:
a) Patient withdrawal of consent;
b) Retreatment;
c) Study termination by sponsor;
d) Lost to follow-up;
e) Death;
f) Other.

### Treatments

SEA-BCMA is a non-fucosylated monoclonal antibody directed against BCMA.

Guidance for intrapatient dose-escalation for patients who have the potential to achieve greater benefit at a dose higher than the dose-level assigned during dose-escalation is described herein.

### Description

SEA-BCMA is a sterile, preservative-free, colorless to light yellow, clear to slightly opalescent solution with no visible particulate matter. SEA-BCMA was supplied in single-dose glass vials. The drug product solution was diluted in sterile 0.9% sodium chloride injection, United States Pharmacopeia (USP), or equivalent, for intravenous (IV) administration.

SEA-BCMA drug product was labeled with a nominal content of 100 mg/vial. Each vial contained 110 mg of SEA-BCMA, which allowed the label quantity to be withdrawn for use. SEA-BCMA drug product consists of SEA-BCMA (20 mg/mL), histidine, arginine, trehalose, and polysorbate 80. The pH of the product was approximately 6.5.

### Dose and Administration

SEA-BCMA will be administered at the assigned dose by IV infusion. SEA-BCMA will not be administered as an IV push or bolus. SEA-BCMA will not be mixed with other medications.

On Cycle 1, Day 1, patients will be closely observed in the clinic for at least 6 hours after completion of study treatment administration during dose escalation. Vital signs will be collected. Additional monitoring for subsequent cycles will be considered upon review of safety data. The observation period after completion of study treatment administration on Cycle 1, Day 1 will be reduced to 2 hours during monotherapy dose expansion and in monotherapy intensive dosing and combination therapy cohorts following review of data from the dose escalation cohort, in which there will be no instances of delayed-onset infusion-related reactions (IRRs).

Infusion duration will vary depending on the method of infusion administration and the SEA-BCMA dose.

The initial approach to SEA-BCMA administration will be stepwise infusion. In a stepwise infusion, the infusion rate will be increased at set time intervals until a defined maximum rate of infusion will be reached. The first infusion of SEA-BCMA will be initiated at a rate of 50 mg/hour. If the first 30 minutes is well-tolerated, the rate will be incrementally increased (no greater than 2-fold increase in rate) every 30 minutes as tolerated until a maximum rate (400 mg/hour) is reached. With subsequent infusions, the infusion rate could be increased more rapidly in shorter time intervals; e.g., after the first 15 minutes, the rate could be incrementally increased (no greater than 2-fold increase in rate) every 15 minutes as tolerated until the maximum rate is reached.

As clinical experience with stepwise infusions evolves, the maximum rate may be increased or decreased based on accumulating safety data and/or recommendations of the SMC. In addition, alternative approaches to SEA-BCMA administration may be evaluated to manage potential safety signals, including IRRs, as recommended by the SMC. These may include systematic implementation of the following strategies: extending the planned infusion duration, fixed-duration infusion (administration at a fixed infusion rate), divided-dose administration, or a change in premedications.

### Fixed-Duration Infusion

Some criteria will be considered regarding fixed-duration infusion:

If fixed-duration infusion is implemented, the SEA-BCMA infusion duration is defined by the physician. As clinical experience with SEA-BCMA infusion evolves, the infusion duration may be increased or decreased based on accumulating safety data and/or recommendations of the SMC.

In an individual patient, if the patient is unable to tolerate the infusion, the infusion duration may be increased; the infusion duration in subsequent infusions may also be increased per investigator discretion with medical monitor approval. Conversely, if a patient does not experience an IRR greater than Grade 1 with consecutive infusions, the infusion duration may be shortened (i.e., administered at a faster rate) at the discretion of the investigator with medical monitor approval, the implementation of which may be dose-cohort specific.

If a fixed infusion rate is implemented, the dose is administered at a fixed rate rather than over a fixed time.

For example, for a fixed infusion rate of 50 mg/hour, a dose of 100 mg would be infused over 2 hours. As clinical experience with administration at a fixed infusion rate evolves, the rate may be increased, or decreased, based on accumulating safety data and/or recommendations of the SMC.

In an individual patient, if the patient is unable to tolerate the infusion rate, the infusion rate may be decreased in subsequent infusions per investigator discretion with medical monitor approval. Conversely, if an individual patient does not experience an IRR greater than Grade 1 with consecutive infusions, the infusion rate may be increased at the discretion of the investigator with medical monitor approval.

### Divided-Dose Administration

Some criteria will be considered regarding divided-dose administration:

If divided-dose administration is implemented, the dose is divided and administered separately within a time period. For example, the dose could be divided in 2 parts, in which the first 10% of the dose is infused over approximately 45 minutes, followed by a 30-minute observation period as the patient remains in the infusion chair. If the investigator determines that the patient has tolerated the initial SEA-BCMA infusion, the remaining 90% is infused over approximately 45 minutes.

### Dose Modifications

On a per-patient basis, lengthening of dosing intervals for toxicity, including DLT, are allowed upon approval by the medical monitor. Patients who experience DLT in Cycle 1 do not receive further treatment with SEA-BCMA, unless clinical benefit is demonstrated with adequately managed toxicity and there is approval from the medical monitor. Examples of clinical benefit include an objective response (OR) assessed by imaging, laboratory assessment, or physical examination; or SD and clinical improvement in disease-related symptoms per investigator. If clinical benefit is demonstrated, the dosing interval is lengthened by 50%-100% after discussion with the medical monitor. The type and severity of the AE observed are taken into consideration to inform the decision. For patients treated at the lowest dose level, the dosing interval may be lengthened, or the patient may be discontinued from treatment.

If a patient has a clinically significant, unresolved AE on the planned dosing day, the dose is delayed for up to 7 days. Dosing delays due to other reasons or lasting >7 days are discussed with the medical monitor; during the DLT period, patients do not receive further treatment with SEA-BCMA unless clinical benefit is demonstrated with adequately managed toxicity and there is approval from the medical monitor. For patients requiring a dose delay >7 days due to an unresolved AE, subsequent doses are reduced or the dosing interval is lengthened by 50-100% after discussion with the medical monitor. Dose delays extending longer than twice the length of the dosing interval require patient discontinuation from study treatment.

In once every 2 weeks (q2wk) dosing, if a patient has a clinically significant, unresolved AE on Day 15 that prevented dosing, the Day 15 visit will be delayed for ≤7 days. On the seventh day, if a patient could not receive the dose, the second dose of the cycle will be eliminated, the Day 15 visit will be skipped, and the Day 22 visit will be performed. If the Day 15 dose is delayed for ≤7 days, study assessments required for Day 15-28 will be delayed by the same number of days as the dose delay, and study drug administration for the next cycle will be delayed by at least the same number of days.

In intensive dosing weekly induction Cycles 1 and 2, if a patient has a clinically significant, unresolved AE that prevents dosing on Day 8, 15, or 22, the dose may be delayed for ≤3 days. On the third day, if a patient cannot receive the dose, the dose of SEA-BCMA will be eliminated and the corresponding visit will be skipped; dosing and visit schedule will resume the following week (e.g. at Day 22, if Day 15 is skipped). However, if a Day 8, 15, or 22 dose is delayed for ≤3 days, subsequent study assessments within the same cycle will be delayed by the same number of days as the dose delay, and study drug administration for the next dose will be delayed by at least the same number of days.

During the DLT period (Cycle 1), growth factor and transfusion support is discouraged unless medically indicated; patients who receive growth factor (e.g., G-CSF or GM-CSF) or transfusion support (other than red blood cell transfusions for MM-related anemia) during this period for reasons other than DLT may not be evaluable for DLT. Consideration is given for growth factor support for prophylaxis or treatment of cytopenias in subsequent cycles (Table 4). During dose escalation, patients with Grade 4 neutropenia have a follow up complete blood count (CBC) with differential obtained 5 days from the time of assessment for evaluation of DLT. In addition, patients with Grade 3 electrolyte abnormalities have a follow up chemistry panel obtained 72 hours from the time of assessment for evaluation of DLT. Serum chemistry and complete blood counts (CBCs) are collected minimally on a weekly schedule during dose delays resulting from toxicity.

Table 4 describes the recommended dose modifications for study treatment-associated toxicity.

TABLE 4
Recommended dose modifications for SEA-BCMA-associated toxicity

| Toxicity | Grade 1 | Grade 2 | Grade 3 | Grade 4 |
|---|---|---|---|---|
| Non-hematologic | Continue at | Continue at | Withhold dose until | Discontinue study |
| (AE or laboratory | same dose | same dose | toxicity is ≤Grade 1 | treatment |
| abnormality) | level | level | or baselineª, and then | |
| | | | resume treatment at | |
| | | | the same dose level | |

| | | | |
|---|---|---|---|
| Hematologic | Continue at | Continue at | First occurrence: Withhold dose until |
| (neutropenia, | same dose | same dose | resolution to ≤Grade 2 or baseline; for Grade 3 |
| thrombocytopenia, | level | level | events, resume treatment at the same dose |
| and anemia) | | | level; for Grade 4 events, either resume |
| | | | treatment at the same dose level after |
| | | | discussion with the medical monitor or |
| | | | discontinue treatment at the discretion of the |
| | | | investigator. Treatment delay of up to 7 days is |
| | | | permitted.ª |
| | | | Second occurrence: Withhold dose until |
| | | | toxicity is ≤Grade 2 or baselineª. Either |
| | | | resume treatment at the same dose level with |
| | | | growth factor support after discussion with the |
| | | | medical monitor or discontinue study treatment |
| | | | at the discretion of the investigator^{b} |
| Infusion-related | See Section I.A.1 | | |
| reaction | | | |

^{a}Treatment delays of >7 days are to be discussed with the medical monitor

Intrapatient dose escalation is permitted in the event that a patient tolerates at least 1 cycle of SEA-BCMA and achieves SD or better. Additional treatment cycles may be administered at 1 dose level below the currently enrolling dose level for dose escalation (or at the MTD if it has been determined).

### Dexamethasone

### Dose and Administration

Dexamethasone will be given on Days 1, 8, 15, and 22 of each 28-day cycle. Dexamethasone will be administered as an IV infusion or orally (PO) at a dose of 40 mg. In combination therapy with nirogacestat and SEA-BCMA, dexamethasone will be administered IV only. The dose of dexamethasone is 20 mg for patients ≥75 years, or with BMI <18.5, or known to be intolerant of dexamethasone 40 mg. On days when SEA-BCMA is administered, dexamethasone is administered 1 to 3 hours prior to the SEA-BCMA infusion.

### Dose Modifications

Dose modifications and supportive care by toxicity are listed in Table 5.

TABLE 5
Dose modifications for dexamethasone-associated toxicity

| CTCAE | | Recommended Dose |
|---|---|---|
| Category | Toxicity | Modification/Supportive Care |
| Gastrointestinal | Grade 1-2 dyspepsia, gastric | Treat with a proton pump inhibitor such as |
| | or duodenal ulcer, gastritis | omeprazole. |
| | requiring medical | If symptoms persist, decrease |
| | management | dexamethasone dose by 50% |
| | ≥Grade 3 requiring | Hold dexamethasone until symptoms are |
| | hospitalization or surgery | adequately controlled. Then, restart at 50% |
| | | of current dexamethasone dose along with |
| | | concurrent therapy with a proton pump |
| | | inhibitor such as omeprazole. If symptoms |
| | | persist despite above measure, discontinue |
| | | dexamethasone and do not resume. |
| | Acute pancreatitis | Discontinue dexamethasone and do not |
| | | resume. |
| Cardiovascular | ≥Grade 3 edema limiting | Diuretics as needed and decrease |
| | function and unresponsive | dexamethasone dose by 25%; if edema |
| | to therapy or anasarca | persists despite above measures, decrease |
| | | dose to 50% of initial dose; discontinue |
| | | dexamethasone and do not resume if |
| | | symptoms persist despite 50% reduction |
| Neurology/ | ≥Grade 2 confusion or | Hold dexamethasone until symptoms |
| Psychiatric | mood alteration interfering | adequately controlled. Restart at 50% of |
| | with function | current dose. If symptoms persist despite |
| | | above measure, discontinue dexamethasone |
| | | and do not resume |
| Musculoskeletal | ≥Grade 2 muscle weakness, | Decrease dexamethasone dose by 25%; if |
| | symptomatic and interfering | weakness persists despite above measures, |
| | with function but not | decrease dose to 50% of initial dose; |
| | interfering with activities of | discontinue dexamethasone and do not |
| | daily living | resume if symptoms persist despite 50% |
| | >Grade 2 muscle weakness, | Hold dexamethasone until muscle weakness |
| | symptomatic and interfering | is ≤Grade 1 or baseline. Then decrease |
| | with activities of daily | dexamethasone dose by 25% and resume; |
| | living | if weakness persists despite above |
| | | measures, decrease dose to 50% of initial |
| | | dose; discontinue dexamethasone and do |
| | | not resume if symptoms persist despite |
| | | 50% |
| Metabolicª | ≥Grade 3 hyperglycemia | Treatment with insulin or oral |
| | | hypoglycemic agents as needed. If |
| | | uncontrolled despite above measure, |
| | | decrease dose by 25% decrements until |
| | | levels are satisfactory |
| Constitutional | ≥Grade 2 insomnia | Decrease dexamethasone dose by 50% |

^{a}Patients who enter the study with elevated hemoglobin A1c (HbA1c) (≥6.5%) or fasting glucose (≥126 mg/dL) at screening must be referred to an appropriate provider for glucose management prior to or within 1 week of starting study treatment in Cycle 1.

### Nirogacestat

### Dose Administration

Nirogacestat will be administered BID at a dose of 100 mg PO on Days 1 to 28 of each 28 day cycle. Patients should take their BID dose orally approximately every 12 hours, without regard to food. If a patient misses a scheduled dose of nirogacestat and it is within 6 hours of the scheduled dose, the patient should immediately administer the missed dose and resume study treatment in accordance with the normal administration schedule. If more than 6 hours have elapsed since the time of scheduled administration, the patient should be instructed not to administer the missed dose and to resume study treatment as prescribed. Patients should not take 2 doses together to “make up” for a missed dose. If a patient vomits any time after taking a dose, then they must be instructed not to take another dose to “make up” for vomiting, but rather to resume subsequent doses as prescribed. If a patient inadvertently takes 1 extra dose, then the patient should not take the next scheduled dose of study treatment. Delivery of nirogacestat via nasogastric tube or gastrostomy tube will not be allowed. The tablets should be swallowed whole with water and not broken or chewed. For doses of 100 mg once a day (QD), doses may be administered within 12 hours of the scheduled missed dose.

### Dose Modifications

Nirogacestat dosing will be interrupted and/or dose reduced for the AEs described in Table 6 and below.

If a patient experiences an AE described in Table 6 that is considered related to nirogacestat, nirogacestat will be held until the event is resolved to Grade ≤1 or baseline, then nirogacestat will be restarted at the reduced dose as described Table 6.

If the AE does not resolve to Grade ≤1 or baseline after holding nirogacestat for 7 days, nirogacestat may be resumed only after discussion with the sponsor.

If the same Grade ≥3 AE recur at the reduced dose, and the AE is considered related to nirogacestat, it may be permanently discontinued following discussion with the sponsor.

TABLE 6
Recommended dose modifications for nirogacestat-related toxicity

| NCI-CTCAE | | Recommended |
|---|---|---|
| Category | Toxicity | Dose Modification |
| Gastrointestinal | Grade ≥3 diarrhea persisting | Decrease dose to |
| Toxicities | for ≥3 days despite maximal | 100 mg QD |
| | medical therapy | |
| | Grade ≥3 nausea persisting | Decrease dose to |
| | for ≥3 days despite maximal | 100 mg QD |
| | medical therapy | |
| | Grade ≥3 vomiting persisting | Decrease dose to |
| | for ≥3 days despite maximal | 100 mg QD |
| | medical therapy | |
| Other toxicities | Grade ≥3 skin toxicity | Decrease dose to |
| | | 100 mg QD |
| | Grade ≥3 hypophosphatemia | Decrease dose to |
| | persisting for ≥7 days despite | 100 mg QD |
| | maximal replacement | |
| | therapy and in the absence | |
| | of symptoms | |
| | Any clinically significant | Decrease dose to |
| | Grade ≥3 non-hematological | 100 mg QD |
| | toxicities | |
| | Anaphylaxis | Permanently |
| | | discontinue |
| | Grade ≥3 hypersensitivity | Permanently |
| | reaction | discontinue |
| | Hepatic toxicities | (Liver Chemistry |
| | | stopping criteria |
| | | for nirogacestat) |

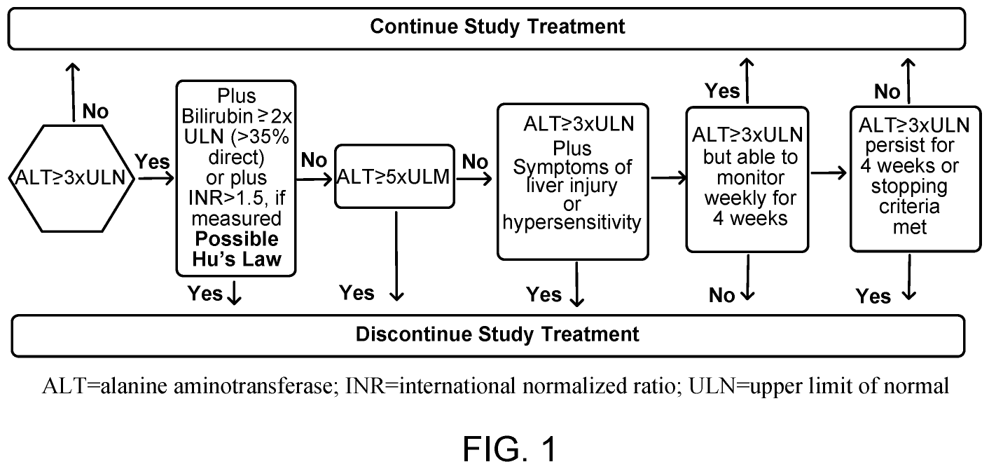

A second dose reduction to 50 mg QD may be permitted after discussion with Sponsor's medical monitor. Additional management of nirogacestat-related toxicities is described below:
Liver Chemistry stopping criteria for nirogacestat: Discontinuation of nirogacestat for abnormal liver function should be considered by the investigator when a patient meets one of the conditions outlined in FIG. 1 or if the investigator believes that it is in the best interest of the patient.

### Management of Nirogacestat Associated Adverse Events

### Anti-Diarrheal, Anti-Emetic Therapy

Primary prophylaxis of diarrhea, nausea and vomiting is permitted in the first cycle. Primary prophylaxis in subsequent cycles is at the investigator's discretion. Events of diarrhea have been commonly reported in patients receiving nirogacestat. Patients experiencing diarrhea considered related to nirogacestat should be treated with loperamide, or other institutional standard of care. The recommended initial dose of loperamide is 4 mg followed by 2 mg after each unformed stool until the diarrhea is controlled, after which the dosage should be reduced to meet individual requirements. Loperamide should be dosed according to the treating physician's medical discretion. Patients should also receive appropriate fluid and electrolyte replacement, including dietary phosphate supplementation, as needed. If diarrhea is Grade ≥3 diarrhea and persists ≥3 days despite maximal medical therapy, nirogacestat should be held until the diarrhea is resolved to Grade ≤1 or baseline, then restarted at a dose of 100 mg QD (Table 6). If the diarrhea does not resolve to Grade ≤1 or baseline after holding study treatment for 7 days, study treatment may be resumed at a reduced dose of 100 mg once a day (QD) only after discussion with the medical monitor.

### Skin Rash

Events of skin rash have been reported in patients receiving nirogacestat.

### Non-Acneiform Rashes Skins Eruptions

Pruritic eruptions/skin rash and other non-acneiform rash should be treated with a moisturizer such as Cerave or Eucerin or another equivalent product. If symptomatic, a low potency topical steroid such as betamethasone valerate lotion (0.05%), desonide cream (0.05%), fluocinolone acetonide solution (0.01%), dexamethasone sodium phosphate cream (0.1%), hydrocortisone acetate cream (1%), methylprednisolone acetate cream (0.25%) or equivalent may also be used.

### Acneiform Rash

Topical clindamycin (0.1%) gel or lotion applied BID, rather than steroids, is the most helpful for pustular rash. In severe cases, semisynthetic oral tetracyclines such as doxycycline or minocycline may also be useful with appropriate precautions in women of child-bearing potential.

### Follicular Cysts

Follicular cysts can be associated with disruptions of the gamma secretase and Notch signaling pathway which help maintain pilosebaceous gland function. This adverse reaction was observed in a phase 2 study of nirogacestat in desmoid patients conducted by the NCI (O'Sullivan Coyne, 2018). If this event is suspected, it is recommended that a dermatology consultation is obtained for appropriate management recommendations.

### Management of Adverse Reaction

### 1. Management of SEA-BCMA Infusion Reactions

IRRs may occur during the infusion of monoclonal antibody therapies such as SEA-BCMA. The infusion should be administered at a site properly equipped and staffed to manage anaphylaxis should it occur. All supportive measures consistent with optimal patient care should be given throughout the study according to institutional standards. Supportive measures may include extending the infusion time and/or administering medications for IRRs.

During dose escalation, additional mitigation strategies may be explored to manage IRRs. These may be implemented upon SMC recommendation, and may include but are not limited to any or all of the following:
Slowing, interruption, or other adjustments in the administration of SEA-BCMA
Potential premedication or postmedication for infusions, for example:
   Antihistamines, such as diphenhydramine 50 mg IV or equivalent and famotidine 40 mg IV or equivalent
   Antipyretics, such as acetaminophen 500-1,000 mg PO
   Antiemetics, such as ondansetron
   IV fluid support, such as normal saline
   Anti-rigor medication, such as meperidine
   Vasopressors
   Corticosteroids, such as hydrocortisone 100 mg IV or equivalent or methylprednisolone 40 mg IV or equivalent (for patients not receiving dexamethasone as combination therapy)

Recommendations for the management of IRRs are detailed in Table 7. IRRs should be graded according to NCI-CTCAE, version 4.03, guidelines.

TABLE 7
Management of infusion-related reactions

| | | | |
|---|---|---|---|
| Grade 1 | Grade 2 | Grade 3 | Grade 4 |

IRR Grade^{a}

| | | | |
|---|---|---|---|
| Mild transient | SEA-BCMA treatment | Prolonged (e.g., not | Life-threatening |
| reaction; | interruption indicated | rapidly responsive | consequences; urgent |
| SEA-BCMA | but | to symptomatic | intervention indicated |
| treatment | responds promptly to | medication and/or brief | |
| interruption not | symptomatic treatment | interruption of infusion); | |
| indicated; | (e.g., | recurrence of symptoms | |
| intervention not | antihistamines, | following initial | |
| indicated | NSAIDS, | improvement; | |
| | narcotics, IV fluids); | hospitalization indicated | |
| | prophylactic | for | |
| | medications | clinical sequelae | |
| | indicated for ≤24 hr | | |

Treatment Recommendations

| | | | |
|---|---|---|---|
| Monitor vital signs | Hold SEA-BCMA | Stop SEA-BCMA | Stop SEA-BCMA |
| more frequently | treatment. Monitor vital | treatment. Institute | treatment immediately. |
| until symptoms | signs more frequently | additional medical | Hospitalization. |
| have resolved and | until symptoms have | management as indicated. | |
| patient is medically | resolved and patient is | Consider hospitalization. | |
| stable. Administer | medically stable. | | |
| symptomatic | Administer | | |
| treatment as | symptomatic treatment | | |
| medically indicated. | as medically indicated. | | |
| | If patient responds | | |
| | promptly and is | | |
| | medically stable in the | | |
| | opinion of the | | |
| | investigator, | | |
| | SEA-BCMA treatment | | |
| | may be continued at a | | |
| | slower rate. | | |

Dose Modifications

| | | | |
|---|---|---|---|
| Consider | Consider premedication | Patients with an IRR that | Permanently |
| premedication with | with subsequent | resolves to baseline or | discontinue from |
| subsequent | SEA-BCMA treatment. | Grade 1 or lower within | study treatment. |
| SEA-BCMA | Consider slower | approximately 2 hours | |
| treatment. | infusion rate. If | after intervention may | |
| | recurrent after the | continue SEA BCMA at | |
| | above measures, | the same dose with | |
| | consider dose | premedications required | |
| | reduction to 1 | prior to all subsequent | |
| | dose level below | doses, if approved by the | |
| | current dose. | medical monitor. | |
| | | OR | |
| | | Permanently discontinue | |
| | | from study treatment. | |

### Per NCI-CTCAE Version 4.03

If anaphylaxis occurs, administration of SEA-BCMA should be immediately and permanently discontinued.

All Grade 3 or 4 events of IRR (with onset during infusion or within ≤24 hr after infusion) or hypersensitivity reaction (with onset occurring >24 hr after infusion) must be reported to the sponsor or designee immediately, regardless of relationship to SEA-BCMA. All Grade 4 events are serious adverse events (SAEs) and are to be reported within the SAE reporting timeframe of 24 hours.

Patients experiencing a ≥Grade 3 IRR or delayed hypersensitivity reaction must have an Infusion/Hypersensitivity Reaction (IHR) Visit and an IHR Follow-up Visit for evaluation and collection of blood samples for analysis of the mechanism of action of the reaction.

### Required Premedication and Postmedication for SEA-BCMA

Routine premedication for infusion reactions should be administered prior to the first dose of SEA-BCMA. However, patients who experienced IRRs receive subsequent treatment with premedication such as antihistamines (e.g., diphenhydramine 50 mg IV or equivalent and famotidine 40 mg IV or equivalent), corticosteroids (e.g., hydrocortisone 100 mg IV or equivalent), or acetaminophen (e.g., 500-1,000 mg PO) at least 30 minutes prior to the infusion. As clinical experience with SEA-BCMA infusions evolves, routine premedication prior to the first dose of study treatment may be instituted, as recommended by the SMC.

There are no required postmedications for SEA-BCMA.

In intensive dosing cohort, dexamethasone combination therapy cohort, and nirogacestat and dexamethasone combination therapy cohort, routine premedication for infusion reactions must be administered prior to SEA-BCMA infusion per the following regimen, unless contraindicated or recommended otherwise by the SMC or medical monitor:

Antipyretic+Antihistamine: administer approximately 45 to 90 minutes prior to SEA BCMA infusion (required for all patients for all doses during Cycle 1 and Cycle 2)
(1) Acetaminophen, oral, 650 to 1000 mg
(2) Diphenhydramine, oral or IV, 25 to 50 mg (or equivalent H1 blocker)

If no IRR (infusion-related reactions) is experienced during Cycle 1 or Cycle 2: one or both premedications may be omitted starting with Cycle 3 Day 1 dose.

If IRR occurs despite acetaminophen+antihistamine

Treat with supportive care based on symptoms.

For monotherapy patients (not receiving Dexamethasone), add:

Methylprednisolone, IV, 100 mg (or equivalent dosage intermediate to long-acting corticosteroid) as required premed 1 to 3 hours prior to next SEA-BCMA infusion. If this infusion is tolerated without IRR, methylprednisolone dose may be reduced to 60 mg (or equivalent dosage of intermediate to long-acting corticosteroid), administered either oral or IV, prior to subsequent doses.

Additional premedications (e.g., H2 blockers or leukotriene inhibitors) may be considered.

For combination patients (receiving Dexamethasone), add:

H2 blocker (famotidine 40 mg IV or equivalent) as required premed 45 to 90 minutes prior to all subsequent SEA-BCMA doses

Additional premedications (e.g., leukotriene inhibitors) may be considered.

### Study Assessments

### Screening/Baseline Assessments

Only patients who met all inclusion and exclusion criteria will be enrolled in this study. Assessments will begin after obtaining a signed informed consent from the patient.

Patient medical history includes a thorough review of significant past medical history, current conditions, any treatment for prior malignancies and response to prior treatment, and any concomitant medications. The number of prior lines of therapy will be determined using the criteria established by Rajkumar et al. (Rajkumar et al., Blood 126(7): 921-2, 2015). In brief:
If a treatment regimen is discontinued for any reason and a different treatment regimen is started, it is considered a new line of therapy.
A new line of therapy is also considered to start when an unplanned substitution or addition of 1 or more drugs is made to an existing course of therapy for any reason.
In patients undergoing >1 ASCT (except in the case of a planned tandem ASCT), each transplant that follows the first one should be considered a new line of therapy.
A planned course of therapy that has multiple phases, such as induction therapy followed by the first ASCT and maintenance therapy, is considered to be a single line of therapy.

A baseline plasmacytoma scan will be conducted during screening only in cases of suspected or known plasmacytoma. During treatment, plasmacytoma evaluations will be performed at any time to confirm a response of PR or better, or as clinically indicated to confirm PD.

Bone marrow aspirate (including a bone marrow aspirate clot) and biopsy will be required as part of the baseline visit.

Physical examinations will include assessments of the following body parts/systems: abdomen, extremities, head, heart, lungs, neck, and neurological. Weight and height will also be measured; measurements of height will be obtained within the prior 12 months may be utilized.

Blood and urine tests will include CBC with differential, serum chemistry panel, serology (hepatitis B and C), PT/PTT/INR, hBA1c (for patients in the combination cohort) and urinalysis. A pregnancy test will be conducted for patients of childbearing potential. Urinalysis with microscopy will be required if urinalysis results would be abnormal. Spot urine for UPC ratio calculation will be sufficient; however, if UPC >2, an additional collection of 24-hour urine for UPC calculation will be required.

Blood samples will be collected for pharmacodynamic biomarker assessments.

### Response/Efficacy Assessments

Response assessment will include SPEP/immunofixation, UPEP/immunofixation (in patients with a baseline urine M protein ≥200 mg/24 hour or for assessment of VGPR or better), SFLC, quantitative immunoglobulins, and plasmacytoma evaluation by imaging (at baseline, every 4 cycles, and at additional time points if clinically indicated). These samples will be collected for local assessment. In addition, blood will be analyzed in the central laboratory using a modified SPEP for patients with IgG myeloma.

Bone marrow aspirate, including a BM aspirate clot, and biopsy will be required as part of the baseline visit, as well as on Day 4 of Cycle 1 (in expansion cohort only, contingent upon activity observed during dose escalation or emerging during dose expansion), Day 22-28 of Cycle 2, and to confirm CR in patients negative for blood and urine M protein. In monotherapy intensive dosing and combination therapy, bone marrow aspirate and biopsy will also be required in Cycle 6 and every 6 cycles thereafter. Both bone marrow aspirate and biopsy samples will be assessed locally at the site for clinical evaluation (with the exception of Cycle 1 Day 4 specimen). In addition, biomarker analyses will be performed centrally on these samples. Any additional bone marrow aspirates and biopsies collected at any other time while on the trial may also be submitted for central assessment.

The bone marrow specimens will be tested centrally for assessment of response/resistance to SEA-BCMA and could include but are not limited to: evaluation of BCMA expression, immune activation, disease risk profiling, gene expression profiling, and minimal residual disease (MRD) assessment.

The determination of antitumor activity will be based on response assessments made according to the 2016 IMWG Criteria (Kumar et al., Lancet Oncol 17(8): e328-46, 2016) and treatment decisions by the investigator will be based on these assessments. Clinical response of sCR, CR, VGPR, PR, SD, and PD will be determined at each assessment based on local laboratory (and the modified SPEP run by the central laboratory for patients with IgG MM), radiological, and clinical evaluations. Progressive disease will be based on IMWG 2016 criteria and/or clinical disease progression per investigator. All IMWG responses will be confirmed responses. When applicable, determination of immunophenotypic CR, MRD status, and minimal response will be made per the IMWG 2016 criteria.

### Pharmacokinetic and Immunogenicity Assessments

Blood and bone marrow samples for PK and ATA assessment will be collected. Qualified assays will be used to measure concentrations of SEA-BCMA in serum and bone marrow and ATA in serum. Remaining PK samples will be archived for possible analysis of SEA-BCMA-related species. The assays will include enzyme-linked immunosorbent assays (ELISA) assay, as well as other assays if further characterization will be required.

A qualified electrochemiluminescence assay will be used to assess ATA.

### Biomarker Studies

Peripheral blood and bone marrow samples for biomarker analyses will be collected at time points outlined in the following sections. In addition to protocol-mandated collections of tumor specimens, bone marrow specimens collected at the discretion of the investigator could be submitted for central biomarkers analysis. For all bone marrow collections, sites will supply bone marrow aspirates as well as bone marrow biopsy specimens and bone marrow aspirate clot specimens as formalin-fixed, paraffin-embedded (FFPE) blocks. For samples acquired for SOC, unstained slides might be submitted if an FFPE block for the bone marrow biopsy or clot were not available. Samples will be sent to the central lab for analysis as described in the laboratory manual.

Samples will be evaluated for expression of BCMA and relevant biomarkers that might be associated with the activity of SEA-BCMA and/or change in response to treatment. Analysis of tumor tissue and peripheral blood could also include markers associated with prognosis, response, or resistance. Changes in peripheral blood immune cell subsets will be measured as potential pharmacodynamic and safety markers.

### Genetic Profiling of Effector Cells

Small nucleotide polymorphisms of FcγRII and FcγRIII, which may influence the response to SEA-BCMA, will be determined, including, but not limited to, testing of the following polymorphisms:
FCGRIIIA-158V/F
FCGRIIA-131H/R
   Serum Free Light Chain and modified SPEP

Kappa and lambda free light chains will be quantified in serum of patients as surrogate markers of antitumor activity.

For patients with IgG myeloma who have low levels of serum M-protein SPEP, a reflex modified SPEP assay will be used to assess for residual serum M-protein in the absence of interference from SEA-BCMA.

### Peripheral Blood Immunophenotyping

Peripheral blood samples will be collected for evaluation of circulating immune cells by flow cytometry. Changes in circulating immune cell subsets will be measured as potential pharmacodynamic markers of SEA-BCMA activity. Flow cytometry measurements will include, but not be limited to, characterizing NK cells, monocytes, T cells, and B cells.

### Plasma Cytokines/Chemokines

The levels of circulating cytokines/chemokines may be assessed by ELISA and/or multiplex cytokine/chemokines assays.

### Soluble Target and Ligands

The levels of circulating soluble BCMA (sBCMA), APRIL and BAFF may be assessed by ELISA or other methods (e.g., LC-MS or flow cytometry).

### Plasma Biomarkers and PBMCs

Plasma and PBMCs will be collected for retrospective analyses of cellular and circulating biomarkers associated with response and/or resistance to SEA-BCMA.

### Characterization of Tumor Tissue

Baseline and on-treatment bone marrow aspirates and biopsies will be collected to assess disease relevant immune subsets, characterize tumor burden, investigate depth of response and determine prognostic signatures and response to treatment. Additional protein, gene expression profiling, as well as further molecular characterization of the tumor for myeloma disease relevant risk markers, may also be evaluated to identify biomarkers predictive of response or resistance to SEA-BCMA.

### Bone Marrow Immunophenotyping

Expression of BCMA on tumor plasma cells, as well as presence and changes of immune components in the bone marrow, may be evaluated by flow cytometry and/or immunohistochemistry.

### Gene Expression Profiling/NGS/FISH

Baseline and treatment-related changes in gene expression profiles in tumor and tumor microenvironment may be assessed by RNA sequencing of tumor (CD138-positive) and non-tumor (CD138-negative) cells purified from bone marrow aspirates, to determine prognostic disease-risk signatures as well as baseline characteristics and on-treatment changes that may correlate with response or resistance. Cytogenetic analyses or DNA sequencing of CD138-positive plasma cells enriched from bone marrow aspirate will be collected at Baseline may also be carried out to further determine genetic changes that may predict or be associated with response to SEA-BCMA.

### MRD

MRD evaluation using the Adaptive NGS for MRD assay (Martinez-Lopez et al., Blood 123(20): 3073-9, 2014) may be carried out on relevant specimens to understand the activity of SEA-BCMA.

### Bone Marrow Plasma

Bone marrow plasma will be collected and may be tested for levels of soluble target, ligands, and/or cytokines/chemokines that may influence or correlate with response to SEA-BCMA.

### Adverse Events

According to the International Council for Harmonisation (ICH) E2A guideline Definitions and Standards for Expedited Reporting, and 21 CFR 312.32, IND Safety Reporting, an AE is any untoward medical occurrence in a patient or clinical investigational subject administered a medicinal product and which does not necessarily have a causal relationship with this treatment.

In general, an abnormal laboratory value should not be recorded as an AE unless it is associated with clinical signs or symptoms, requires an intervention, results in a SAE, or results in study termination or interruption/discontinuation of study treatment (SEA-BCMA and/or dexamethasone). When recording an AE resulting from a laboratory abnormality, the resulting medical condition rather than the abnormality itself should be recorded (e.g., record “anemia” rather than “low hemoglobin”).

### Serious Adverse Events

An AE was classified as an SAE if it met one of the following criteria:
Fatal: AE resulted in death
Life threatening: The AEs placed the patient at immediate risk of death. This classification does not apply to an AE that hypothetically might cause death if it were more severe.
Hospitalization: The AE resulted in hospitalization or prolonged an existing inpatient hospitalization. Hospitalizations for elective medical or surgical procedures or treatments planned before the signing of informed consent in the study or routine check-ups are not SAEs by this criterion. Admission to a palliative unit or hospice care facility is not considered to be a hospitalization. Hospitalizations or prolonged hospitalizations for scheduled therapy of the underlying cancer or study target disease need not be captured as SAEs.
Disabling/incapacitating: An AE that resulted in a persistent or significant incapacity or substantial disruption of the patient's ability to conduct normal life functions.
Congenital anomaly or birth defect: An adverse outcome in a child or fetus of a patient exposed to the molecule or study treatment regimen before conception or during pregnancy.
Medically significant: The AE did not meet any of the above criteria, but could have jeopardized the patient and might have required medical or surgical intervention to prevent one of the outcomes listed above or involves suspected transmission via a medicinal product of an infectious agent. Potential drug-induced liver injury (DILI) also is considered a medically significant event.

### Adverse Event Severity

AE severity will be graded using the NCI-CTCAE, version 4.03.

AE severity and seriousness will be assessed independently. ‘Severity’ characterizes the intensity of an AE. ‘Serious’ is a regulatory definition and serves as a guide to the sponsor for defining regulatory reporting obligations.

### Relationship of the Adverse Event to Study Treatment

The relationship of each AE to each study treatment (SEA-BCMA and/or dexamethasone) will be evaluated by the investigator using the following criteria:
Related: There is evidence to suggest a causal relationship between the drug and the AE, such as:
   A single occurrence of an event that is uncommon and known to be strongly associated with drug exposure (e.g., angioedema, hepatic injury, Stevens-Johnson Syndrome)
   One or more occurrences of an event that is not commonly associated with drug exposure, but is otherwise uncommon in the population exposed to the drug (e.g., tendon rupture)
Unrelated: Another cause of the AE is more plausible (e.g., due to underlying disease or occurs commonly in the study population), or a temporal sequence cannot be established with the onset of the AE and administration of the study treatment, or a causal relationship is considered biologically implausible

### Data Analysis Methods

### Determination of Sample Size

Approximately 305 patients will be enrolled in this study. This number is based on the following. Approximately 65 patients were enrolled in SEA-BCMA monotherapy studies. This number was based on the assumption that approximately 25 patients were evaluated in dose-escalation and that approximately 40 patients were evaluated in an expansion cohort at the MTD or optimal dose to further define the safety and antitumor activity of SEA-BCMA.

Operating characteristics of the dose escalation part of the study, including the average number of patients allocated to each dose across a variety of toxicity scenarios are presented in the simulation report.

Approximately 40 patients will be enrolled in the combination therapy studies, including each of the Cohorts 1 and 2). An interim analysis will be performed after 20 patients will be efficacy-evaluable at optimal dose in each cohort, to determine whether the cohort could be further expanded to 40 patients.

No formal hypothesis test is planned for the expansion cohorts. Assuming a 30% ORR, the 95% exact confidence interval (CI) is (17%, 47%) and the 80% exact CI is (20%, 41%) with 40 patients.

No formal hypothesis is planned for intensive dosing monotherapy and combination therapy. Assuming the observed ORR is between 30%-50%, the 95% binomial exact CIs are summarized in Table 8 below.

TABLE 8
95% binomial exact CIs

| | 95% CI | 95% CI |
|---|---|---|
| ORR | (N = 20) | (N = 40) |
| 30% | 12%, 54% | 17%, 47% |
| 40% | 19%, 64% | 25%, 57% |
| 50% | 27%, 73% | 34%, 66% |
| 60% | 36%, 81% | 43%, 75% |
| 70% | 46%, 88% | 54%, 83% |

### Objective Response Rate

A patient is determined to have an OR if, based on the 2016 IMWG uniform response criteria, they achieve a sCR, CR, VGPR, or a PR. The ORR will be defined as the proportion of patients with an OR per investigator. Patients whose disease response could not be evaluated per the 2016 IMWG uniform response criteria will be scored as Not Evaluable for calculating the ORR. Patients who do not have post baseline response assessment, or the response is Not Evaluable per IMWG criteria will be counted as non-responders in calculation of ORR.

### Complete Response Rate

A patient is determined to have a CR if, based on the 2016 IMWG uniform response criteria they achieve a sCR or CR. The CR rate is defined as the proportion of patients with a CR per investigator. Patients whose disease response cannot be evaluated per the IMWG uniform response criteria will be scored as Not Evaluable for calculating the CR rate.

### Duration of Objective Response

Duration of OR is defined as the time from first documentation of OR (sCR, CR, VGPR, or PR) to the first documentation of disease progression or to death due to any cause, whichever comes first. Disease progression includes objective evidence of tumor progression (based on serum, urine, or bone marrow assessments) and/or clinical progression per investigator. Duration of response will be censored on the date of the last disease assessment documenting absence of PD for patients who do not have disease progression and are still on study at the time of an analysis, or are removed from study prior to documentation of tumor progression. Patients who have started a new antitumor treatment prior to documentation of PD will be censored at the last disease assessment prior to start of new treatment.

Duration of response will only be calculated for the subgroup of patients achieving a sCR, CR, VGPR, or PR.

### Duration of Complete Response

Duration of CR is defined as the time from first documentation of complete response (sCR, CR) to the first documentation of disease progression or to death due to any cause, whichever comes first. Disease progression includes objective evidence of tumor progression (based on serum, urine or bone marrow assessments) and/or clinical progression per investigator. Duration of CR will be censored on the date of the last disease assessment documenting absence of PD for patients who do not have disease progression and were still on study at the time of an analysis, or were removed from study prior to documentation of tumor progression. Patients who have started a new antitumor treatment prior to documentation of PD will be censored at the last disease assessment prior to start of new treatment.

Duration of CR will only be calculated for the subgroup of patients achieving a sCR or CR.

### Progression-Free Survival

PFS is defined as the time from the start of any study treatment to first documentation of disease progression or to death due to any cause, whichever comes first. Disease progression includes objective evidence of tumor progression (based on serum, urine or bone marrow assessments) and/or clinical progression per investigator. PFS will be censored on the date of the last disease assessment documenting absence of progressive disease (PD) for patients who do not have disease progression and will still be on study at the time of an analysis, or are removed from study prior to documentation of tumor progression. Patients who have started a new antitumor treatment prior to documentation of PD will be censored at the last disease assessment prior to start of new treatment. Patients lacking an evaluation of tumor response after their first dose will have their event time censored at 1 day.

### Overall Survival

OS is defined as the time from the start of any study treatment to the date of death due to any cause. Specifically: OS=date of death−date of first dose of any study treatment+1.

OS for patients who were alive at their date of last contact, including those lost to follow-up, will be censored at the date of last contact. If the last recorded date where a patient was known to be alive was the date of first dose of any study treatment, survival time would be censored on the date of first dose of any study treatment (i.e., OS duration of 1 day).

### MRD-Negativity Rate

The rate of MRD negativity will be reported among patients who achieved VGPR or better.

### Efficacy Analyses

All efficacy analyses will be presented using the All Treated Patients set. Selected efficacy endpoints will also be presented using the EE analysis set. The observed ORR and CR rate and corresponding 95% Cis will be presented. Patients whose disease response cannot be assessed will be counted as non-responders. Patients with intrapatient dose escalation prior to achieving a response will be counted as non-responders at their initial dose.

Duration of response, PFS and OS will be estimated using Kaplan-Meier methodology, and Kaplan-Meier plots will be provided. Medians will be calculated, where possible. The 95% CIs will also be calculated, as appropriate.

### Pharmacokinetic and Immunogenicity Analyses

The PK of SEA-BCMA will be evaluated by noncompartmental analysis. The following PK parameters will be determined where data allow:
Area under the curve
Concentration at the end of infusion (Cₑₒᵢ) or maximum observed concentration (Cₘₐₓ)
Trough concentration (C_{trough})
Terminal or apparent terminal half-life (t_{1/2})
Systemic clearance and volume of distribution at steady state
Accumulation ratio

### Biomarker Analyses

Peripheral blood and bone marrow aspirates and biopsies will be collected for biomarker assessments. Assessments will be performed with these samples included, but are not limited to, myeloma cell monitoring and profiling, including expression of BCMA and assessments of immune cell populations. Additionally, bone marrow samples will be analyzed to identify gene expression profiles, cytogenetic abnormalities, genetic mutations, and other tumor and tumor microenvironment-related biomarkers that may define disease risk profiles, predict response to SEA-BCMA, and clarify SEA-BCMA mechanisms of action. MRD will be analyzed in selected bone marrow specimens using next generation sequencing (NGS). Plasma and serum will also be collected for quantification of biomarkers of drug activity, which included sFLC, cytokines/chemokines, soluble BCMA, and other soluble biomarkers.

Relationships of biomarker and pharmacodynamic parameters (e.g., baseline values, absolute and relative changes from baseline) to efficacy, safety and PK parameters will be explored. Relationships and associated data that are determined to be of interest will be summarized.

### Example 2. SEA-BCMA Displays Enhanced Activity in the Presence of Gamma Secretase Inhibition

Gamma secretase inhibitors (GSIs) have been shown to block BCMA cleavage and thus increase BCMA expression on the surface of cells (Laurent S A, Hoffmann F S, Kuhn P H, et al. γ-Secretase directly sheds the survival receptor BCMA from plasma cells. Nat Commun. 2015; 6:7333). The current inventors hypothesized that gamma secretase inhibition can improve SEA-BCMA activity on multiple myeloma (MM) cells. As shown in the experiments below, treatment of MM cells in vitro with nirogacestat (purchased from SelleckChem) or DAPT (EMD Millipore), or some other GSIs, enhances SEA-BCMA FcγRIII engagement and antibody dependent cellular cytotoxicity (ADCC). These data suggest the combination of SEA-BCMA with GSI inhibitors in the clinic can lead to greater anti-myeloma activity. Nirogacestat also increases BCMA NF-kB signaling, likely due to increased BCMA expression. SEA-BCMA can block this increased BCMA signaling in MM cells. These data collectively suggest that blocking of proliferative cell signaling by SEA-BCMA can contribute to anti-myeloma activity even in the presence of GSIs in the clinic.

### SEA-BCMA Displays Enhanced FcγRIII Activation in the Presence of Gamma Secretase Inhibition

Experiments were first performed to test the impact of GSIs on the primary mechanism of action of SEA-BCMA, namely ADCC activity. The induction of FcγRIII signaling that initiates ADCC when SEA-BCMA is combined with immune effectors was examined first.

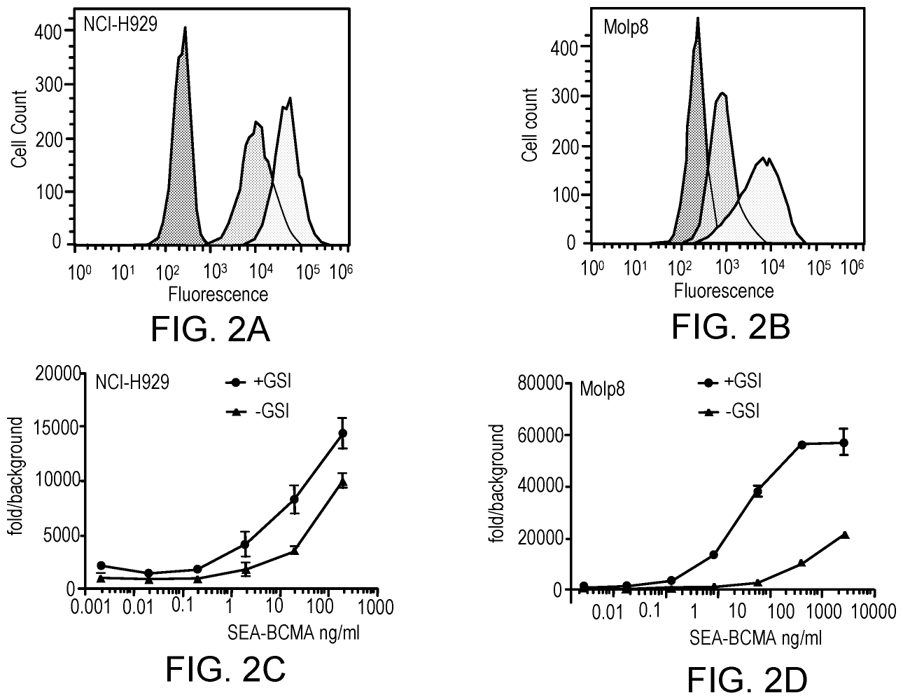

NCI-H929 or Molp-8 MM target cells were incubated with and without 1 μM DAPT for 24 hrs. Cells displayed increased BCMA expression using flow cytometry after incubation with DAPT (FIGS. 2A-2B). FcγRIII signaling was determined using a surrogate assay as manufacturer describes (Promega ADCC reporter bioassay cat #G9302). Cells were bound with antibody dose titrations+/−GSI for 30 minutes at 37° C. CD16A-Jurkat effector cells were then added with a 6:1 effector-to-target cell ratio. After an overnight incubation, the assay was developed with Bio-Glo and relative luminescence units (RLU) were measured on an Envision plate reader. Increased FcγRIII signaling was observed in the presence of the GSI (FIGS. 2C-2D). Thus, as shown in FIGS. 2A-2D, DAPT treatment can induce increased BCMA expression and increased FcγRIII signaling on NCI-H929 and Molp-8 cells. SEA-BCMA is a nonfucosylated antibody that displays enhanced FcγRIII binding affinity and induced signaling in comparison to fucosylated anti-BCMA antibodies. Enhanced signaling is the first step in the primary mechanism of action of SEA-BCMA. This signaling can translate to increased anti-MM cell lysis through ADCC. These data indicate that a GSI can potentially improve SEA-BCMA clinical activity.

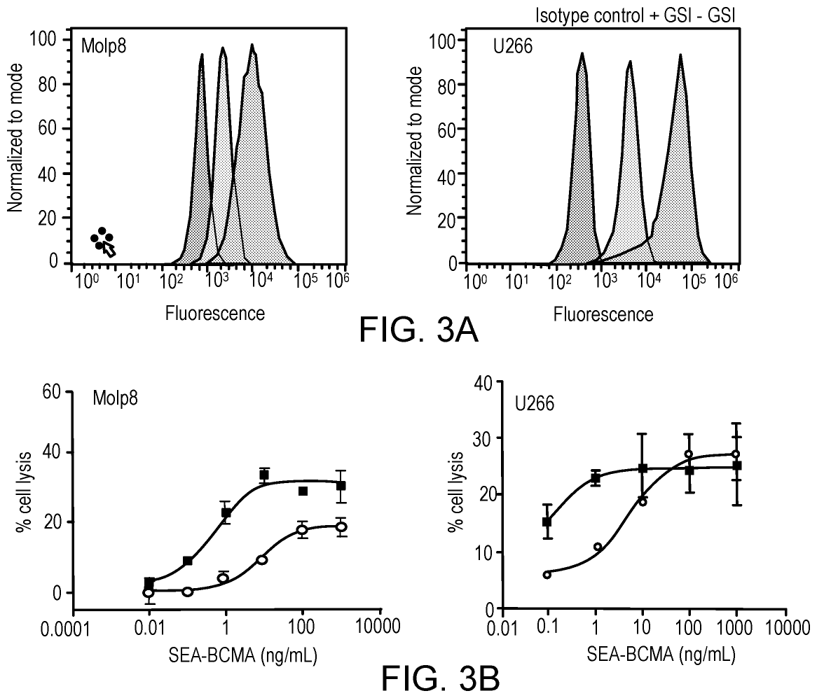

Multiple myeloma cells incubated with and without 0.2 μM nirogacestat GSI for 24 hours also showed increased BCMA expression (FIG. 3A). This translated to increased ADCC when NK effector cells enriched from normal donor PBMC were combined with nirogacestat treated multiple myeloma cells (FIG. 3B). Increased ADCC was particularly notable with multiple myeloma cells that expressed low levels of BCMA, such as MOLP-8 that expressed only 2,000 copies of BCMA. These pre-clinical data supported the combination of SEA-BCMA with nirogacestat in clinical studies.

### SEA-BCMA Displays Enhanced ADCC in the Presence of Gamma Secretase Inhibition

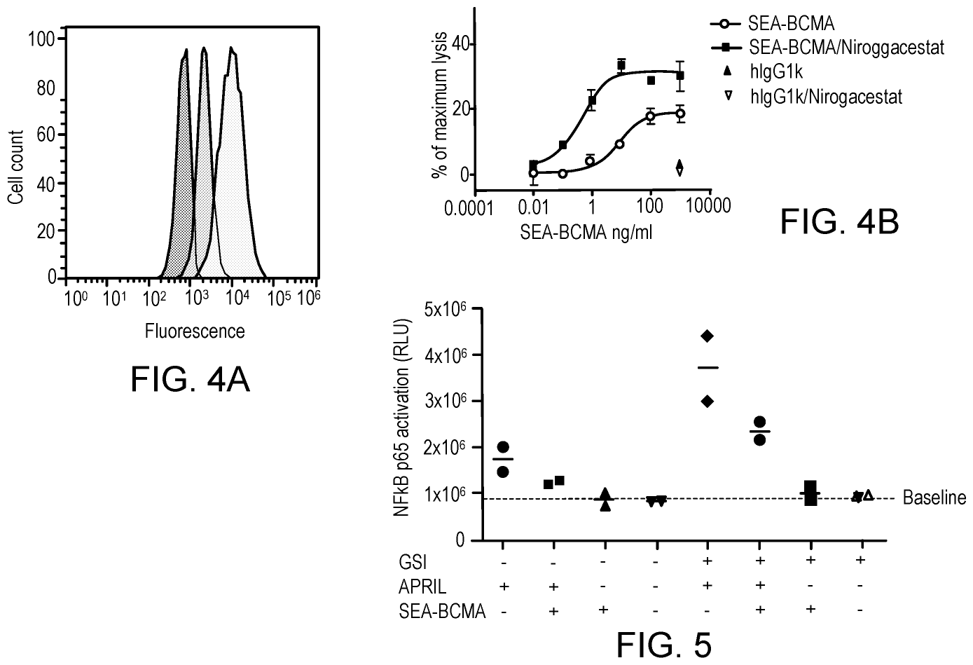

Whether the enhanced FcγRIII signaling translated to increased lysis of MM target cells by SEA-BCMA was determined. Molp-8 MM target cells were incubated with and without 0.2 μM Nirogacestat (purchased from SelleckChem) for 24 hrs. Cells were then Na₂ [⁵¹Cr] O₄ labeled and added to titrations of SEA-BCMA, or isotype antibody control. Effector cells, NK cells enriched from normal donor PBMC, were added at an effector-to-target cell ratio of 10:1 (50,000:5000). Donor NK cells were of the high affinity FcγRIII V/V genotype. The combination of antibodies, NK cells and target cells were incubated for 4h at 37° C. with and without Nirogacestat. The radioactivity released into the culture supernatant of lysed target cells was then measured and the percent specific cell lysis calculated. U266 displayed increased BCMA expression using flow cytometry after incubation with Nirogacestat (FIG. 4A). Increased ADCC was observed in the presence of the GSI (FIG. 4B). This is the primary of mechanism of action of SEA-BCMA and translates to specific enhanced anti-MM lysis. It is expected that this will translate to improvements in anti-MM activity in the clinic when SEA-BCMA is combined with GSIs.

### Example 3. SEA-BCMA Partially Blocks NF-κB Signaling Induced by Gamma Secretase Inhibition

The secondary mechanism of action of SEA-BCMA is to block BCMA proliferative cell signaling. Increased BCMA from GSI treatment is expected to induce increased BCMA signaling. Therefore, the block of this enhanced signaling by SEA-BCMA was tested. BCMA-expressing NCI-H929 cells were serum-starved with and without 0.2 μM Nirogacestat (purchased from SelleckChem) for 16 hrs. Cells were then bound with and without 20 μg/mL SEA-BCMA and incubated with and without 1 μg/ml recombinant human APRIL (R&D Systems) for 20 minutes at 37° C. in the presence or absence of 0.2 μM Nirogacestat. 1 μg of nuclear extract was assayed in duplicate for NF-κB p65 activity by ELISA (TransAM NFκB Chemi p65, Active Motif). Relative Luminescence Units (RLU) were plotted showing increased NF-κB signaling from the GSI, Nirogacestat, which can be partially blocked by SEA-BCMA (FIG. 5). These data suggest that SEA-BCMA can continue to block MM proliferative signaling in the presence of GSIs in the clinic.

1. A method of treating a subject having multiple myeloma (MM), the method comprising administering to the subject: (i) one or more doses of an antibody, or antigen-binding fragment thereof, that specifically binds to a B cell maturation antigen (BCMA), and (ii) one or more doses of nirogacestat, and wherein:
the one or more doses of the antibody or antigen-binding fragment thereof are independently administered to the subject at about 100 mg of the antibody or antigen-binding fragment thereof to about 2,000 mg of the antibody or antigen-binding fragment thereof, and
the one or more doses of nirogacestat are independently administered to the subject at about 80 mg to about 120 mg of nirogacestat.

2. The method of claim 1, wherein the antibody or antigen-binding fragment thereof is a non-fucosylated antibody or antigen-binding fragment thereof.

3. (canceled)

4. The method of claim 1, wherein the antibody or antigen-binding fragment thereof, comprises:
a heavy chain variable region comprising a CDR1 comprising SEQ ID NO: 1, a CDR2 comprising SEQ ID NO: 2, and a CDR3 comprising SEQ ID NO: 3, and
a light chain variable domain comprising a CDR1 comprising SEQ ID NO: 5, a CDR2 comprising SEQ ID NO: 6, and a CDR3 comprising SEQ ID NO: 7.

5. The method of claim 1, wherein the antibody or the antigen-binding fragment thereof comprises a heavy chain variable domain comprising an amino acid sequence that is at least 80% identical to SEQ ID NO: 4 and a light chain variable domain comprising an amino acid sequence that is at least 80% identical to SEQ ID NO: 8.

6. The method of claim 5, wherein the antibody or the antigen-binding fragment thereof comprises a heavy chain variable domain comprising an amino acid sequence that is at least 90% identical to SEQ ID NO: 4 and a light chain variable domain comprising an amino acid sequence that is at least 90% identical to SEQ ID NO: 8.

7. The method of claim 6, wherein the antibody or the antigen-binding fragment thereof comprises a heavy chain variable domain comprising an amino acid sequence of SEQ ID NO: 4 and a light chain variable domain comprising an amino acid sequence of SEQ ID NO: 8.

8. The method of claim 1, wherein the antibody or the antigen-binding fragment thereof is humanized.

9. The method of claim 1, wherein the antibody is an IgG1 antibody.

10.-22. (canceled)

23. The method of claim 1, wherein a single dose of the antibody or antigen-binding fragment thereof is administered to the subject.

24. The method of claim 1, wherein two or more doses of the antibody or antigen-binding fragment thereof are independently administered to the subject.

25. The method of claim 24, wherein the two or more doses of the antibody or the antigen-binding fragment thereof are independently administered to the subject at a frequency of between once a week and about once every four weeks.

26.-37. (canceled)

38. The method of claim 24, wherein the two or more doses of the antibody or the antigen-binding fragment thereof comprise (1) one or more induction doses that are independently administered to the subject during an induction phase and (2) one or more maintenance doses of the antibody or the antigen-binding fragment thereof that are independently administered to the subject during a maintenance phase after the induction phase.

39. (canceled)

40. The method of claim 38, wherein two or more induction doses are independently administered to the subject.

41.-45. (canceled)

46. The method of claim 38, wherein each of the induction dose(s) comprise(s) about 100, about 200, about 400, about 800, or about 1600 mg of the antibody or antigen-binding fragment thereof.

47.-49. (canceled)

50. The method of claim 38, wherein two or more maintenance doses are independently administered to the subject.

51. The method of claim 50, wherein each of the two or more maintenance doses are independently administered to the subject once every 1-4 weeks.

52.-60. (canceled)

61. The method of claim 1, wherein the dose(s) of the antibody or antigen-binding fragment thereof are administered intravenously to the subject.

62. The method of claim 1, wherein a single dose of nirogacestat is administered to the subject.

63. The method of claim 1, wherein two or more doses of nirogacestat are independently administered to the subject.

64. The method of claim 1, wherein each dose of nirogacestat comprises about 100 mg of nirogacestat.

65. The method of claim 63, wherein the two or more doses of nirogacestat are independently administered to the subject at a frequency of about once a day to about four times a day.

66.-67. (canceled)

68. The method of claim 1, wherein the dose(s) of nirogacestat is/are orally administered to the subject.

69. The method of claim 1, wherein the method further comprises independently administering one or more doses of dexamethasone to the subject.

70. The method of claim 69, wherein the method comprises administering a single dose of dexamethasone to the subject or independently administering two or more doses of dexamethasone to the subject.

71.-120. (canceled)

121. A kit comprising:
(a) one or more doses of a pharmaceutical composition comprising an antibody, or antigen-binding fragment thereof, that specifically binds to a B cell maturation antigen (BCMA), wherein the antibody or antigen-binding fragment thereof, comprises: a heavy chain variable region comprising a CDR1 comprising SEQ ID NO: 1, a CDR2 comprising SEQ ID NO: 2, and a CDR3 comprising SEQ ID NO: 3, and a light chain variable domain comprising a CDR1 comprising SEQ ID NO: 5, a CDR2 comprising SEQ ID NO: 6, and a CDR3 comprising SEQ ID NO: 7; and
(b) instructions for performing a method of claim 1.

122. The kit of claim 121, wherein the kit further comprises one or more doses of a pharmaceutical composition comprising nirogacestat and/or one or more doses of a pharmaceutical composition comprising dexamethasone.

123. (canceled)


SEQUENCE LISTING (16 sequences)

SEQ ID NO: 1 - protein, 5 aa, synthetic construct
DYYIH

SEQ ID NO: 2 - protein, 17 aa, synthetic construct
YINPNSGYTNYAQKFQG

SEQ ID NO: 3 - protein, 12 aa, synthetic construct
YMWERVTGFFDF

SEQ ID NO: 4 - protein, 121 aa, synthetic construct
QVQLVQSGAEVKKPGASVKLSCKASGYTFTDYYIHWVRQAPGQGLEWIGYINPNSGYTNYAQKFQGRATMTADKSINTAYVELSRLRSDDTAVYFCTRYMWERVTGFFDFWGQGTMVTVSS

SEQ ID NO: 5 - protein, 11 aa, synthetic construct
LASEDISDDLA

SEQ ID NO: 6 - protein, 7 aa, synthetic construct
TTSSLQS

SEQ ID NO: 7 - protein, 9 aa, synthetic construct
QQTYKFPPT

SEQ ID NO: 8 - protein, 108 aa, synthetic construct
DIQMTQSPSSVSASVGDRVTITCLASEDISDDLAWYQQKPGKAPKVLVYTTSSLQSGVPSRFSGSGSGTDFTLTISSLQPEDFATYFCQQTYKFPPTFGGGTKVEIKR

SEQ ID NO: 9 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 10 - DNA, 994 bp, Homo sapiens
  source 1..994: mol_type = other DNA
aagactcaaacttagaaacttgaattagatgtggtattcaaatccttagctgccgcgaagacacagacagcccccgtaagaacccacgaagcaggcgaagttcattgttctcaacattctagctgctcttgctgcatttgctctggaattcttgtagagatattacttgtccttccaggctgttctttctgtagctcccttgttttctttttgtgatcatgttgcagatggctgggcagtgctcccaaaatgaatattttgacagtttgttgcatgcttgcataccttgtcaacttcgatgttcttctaatactcctcctctaacatgtcagcgttattgtaatgcaagtgtgaccaattcagtgaaaggaacgaatgcgattctctggacctgtttgggactgagcttaataatttctttggcagttttcgtgctaatgtttttgctaaggaagataaactctgaaccattaaaggacgagtttaaaaacacaggatcaggtctcctgggcatggctaacattgacctggaaaagagcaggactggtgatgaaattattcttccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctaggtaattaaccatttcgactcgagcagtgccactttaaaaatcttttgtcagaatagatgatgtgtcagatctctttaggatgactgtatttttcagttgccgatacagctttttgtcctctaactgtggaaactctttatgttagatatatttctctaggttactgttgggagcttaatggtagaaacttccttggtttcatgattaaactcttttttttcctga

SEQ ID NO: 11 - DNA, 363 bp, synthetic construct
  source 1..363: mol_type = other DNA
caagtgcagctggtgcagtccggagcggaagtgaagaaacctggggcgtccgtgaagctcagctgcaaggcctccggctacactttcaccgattactacatccactgggtcagacaggcaccgggacagggactggagtggattggttacatcaaccccaactccgggtacaccaattacgcccagaagttccagggtcgggctacgatgaccgccgacaagtcgatcaacactgcctacgtggaactgtcaaggctgcggtccgatgacaccgccgtgtacttctgtacccgctatatgtgggagcgcgtgactggatttttcgacttctggggccaaggcaccatggtcaccgtgtcgagc

SEQ ID NO: 12 - DNA, 324 bp, synthetic construct
  source 1..324: mol_type = other DNA
gacattcagatgacccagtccccctcgtccgtgtccgcttccgtgggagatcgcgtgaccatcacttgtcttgcgtccgaggatatctcagacgacctggcctggtaccagcagaagcctggaaaggccccgaaggtcctggtgtacactaccagcagcctccagtcgggcgtgccttcacggttctccggttcggggtctggcaccgacttcaccctgactattagctccctgcaacccgaggacttcgccacctacttttgccagcaaacctacaagttcccgccaacgttcggagggggcaccaaggtcgaaatcaaacgt

SEQ ID NO: 13 - protein, 451 aa, synthetic construct
QVQLVQSGAEVKKPGASVKLSCKASGYTFTDYYIHWVRQAPGQGLEWIGYINPNSGYTNYAQKFQGRATMTADKSINTAYVELSRLRSDDTAVYFCTRYMWERVTGFFDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 1353 bp, synthetic construct
  source 1..1353: mol_type = other DNA
caagtgcagctggtgcagtccggagcggaagtgaagaaacctggggcgtccgtgaagctcagctgcaaggcctccggctacactttcaccgattactacatccactgggtcagacaggcaccgggacagggactggagtggattggttacatcaaccccaactccgggtacaccaattacgcccagaagttccagggtcgggctacgatgaccgccgacaagtcgatcaacactgcctacgtggaactgtcaaggctgcggtccgatgacaccgccgtgtacttctgtacccgctatatgtgggagcgcgtgactggatttttcgacttctggggccaaggcaccatggtcaccgtgtcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagaaggttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 15 - protein, 214 aa, synthetic construct
DIQMTQSPSSVSASVGDRVTITCLASEDISDDLAWYQQKPGKAPKVLVYTTSSLQSGVPSRFSGSGSGTDFTLTISSLQPEDFATYFCQQTYKFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - DNA, 642 bp, synthetic construct
  source 1..642: mol_type = other DNA
gacattcagatgacccagtccccctcgtccgtgtccgcttccgtgggagatcgcgtgaccatcacttgtcttgcgtccgaggatatctcagacgacctggcctggtaccagcagaagcctggaaaggccccgaaggtcctggtgtacactaccagcagcctccagtcgggcgtgccttcacggttctccggttcggggtctggcaccgacttcaccctgactattagctccctgcaacccgaggacttcgccacctacttttgccagcaaacctacaagttcccgccaacgttcggagggggcaccaaggtcgaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt